(19) United States
(12) Patent Application Publication
(10) Pub. No.: US 2026/0259127 A1
(43) Pub. Date: Sep. 3, 2026

(54) METHOD AND MARKER FOR ANALYZING A SAMPLE

(71) Applicant: Leica Microsystems CMS GmbH, Wetzlar (DE)

(72) Inventor: Soeren ALSHEIMER, Wetzlar (DE)

(21) Appl. No.: 19/131,566

(22) PCT Filed: Sep. 15, 2023
(86) PCT No.: PCT/EP2023/075470

§ 371 Date: May 21, 2025

(30) Foreign Application Priority Data
Nov. 22, 2022 (WO) PCT/EP2022/082886

(51) Int. Cl. G01N 15/14; G01N 1/30; G01N 15/1429; G01N 33/53

(52) U.S. Cl. CPC G01N 15/147; G01N 1/30; G01N 15/1429; G01N 33/5308; G01N 2015/1477; G01N 2458/10

(57) ABSTRACT

A linker is provided. The linker is configured to bind to an affinity reagent. The linker includes at least one binding site configured to bind at least one dye. The linker further includes an oligonucleotide attachment portion. The oligonucleotide attachment portion is configured to bind to an oligonucleotide barcode of the affinity reagent.

### CROSS REFERENCE TO RELATED APPLICATIONS

This application is a U.S. National Phase application under 35 U.S.C. § 371 of International Application No. PCT/EP2023/075470, filed on Sep. 15, 2023, and claims benefit to PCT/EP2022/082886, filed on Nov. 22, 2022. The International Application was published in English on May 30, 2024 as WO 2024/110088A1 under PCT Article 21(2).

### FIELD

Embodiments of the invention relate to a method for analyzing a sample, in particular a biological sample. Further, embodiments of the invention relate to a device for analyzing a biological sample. The method or the device may also be used for a chemical compound or a chemical element. Furthermore, embodiments of the invention relate to a linker that is configured to bind to an affinity reagent, a reporter comprising a linker and at least one dye, and a marker comprising an affinity reagent and a reporter.

### BACKGROUND

In order to address key problems in the field of life sciences it is vital to precisely detect the presence of an analyte or molecular target in one of a biological sample (e.g. tissue samples or cell cultures), an environmental sample (a soil sample), a water sample, a diagnostic procedure (e.g. in a solid or liquid biopsy or a sample prepared from either) or a lysate or extract from such a sample. This can be done by introducing markers into the sample that bind to specific structures, e.g. specific biomolecules. These markers typically comprise an affinity reagent that attaches to the structure in question and a fluorescent dye that is either directly conjugated to the affinity reagent or attached to the affinity reagent by means of a secondary affinity reagent. There are various techniques for analyzing biological samples prepared in this way. The plexing level in fluorescence microscopy, i.e. the number of different fluorescent dyes that can be read out at the same time is generally low and in the case of fluorescence microscopy typically in the range of 1-5 dyes for channel-based readouts and 5-12 dyes for readouts with spectral detectors, which use dispersive optical elements, such as prisms or gratings in combination with multiple detectors or array detectors. In fluorescence-based cytometry and sorting, somewhat higher plexing levels have been attained, but also in this case plexing is limited to a low number of dyes and consequently markers that can be readout in one experiment.

“Fluorescent cell barcoding” is a multiplexing technique developed by Krutzig and Nolan 2006 and is based on using different mixtures of three fluorescent dyes as described in Nat Methods. 2006 May; 3(5):361-8. doi: 10.1038/nmeth872.

A quote from Tsai et al. 2020 states that “Fluorescent cell barcoding (FCB) is a multiplexing technique for high-throughput flow cytometry (FCM). Although powerful in minimizing staining variability, it remains a subjective FCM technique because of inter-operator variability and differences in data analysis” (J Immunol Methods 2020 February; 477:112667.doi: 10.1016/j.jim.2019.112667. Epub 2019 Nov. 11.) Both the subjectiveness of the technique and inter-operator variability of this method are inherently related to the fact that it is based on encoding a part of the information in the hues of dyes, i.e. in intensity variations like in for example light green, green, dark-green, which severely limits the use of this technique.

However, no technique allows for the fluorescence-based readout of a high number of different markers, wherein readout may refer to image-based or non-image-based readouts.

Fluorescence microscopy allows for imaging the sample with high spatial resolution but involves only a low number of different fluorescent dyes, typically between 1 and 5. The available markers have to accommodate markers that are used to identify cell types, functional markers like protein-of-interest, and general morphological markers in the same experiment. This means that cell types in most imaging experiments are merely poorly identified. This means that rather broad multi cell type populations are being studied, which severely limits the predictive power and translational value of the results generated. While modern approaches exist that allow for a much more reliable and robust identification of cell types, e.g. based on the analysis of genetic regulatory networks (GRNs), they require a much higher number of different markers to be readout from the sample.

While in the adjacent field of cytometry, mass cytometry and imaging mass cytometry techniques can distinguish between around 12 to 30 different markers, they do so with a low spatial resolution.

Spatial profiling techniques can distinguish a number of different markers several orders of magnitude higher, albeit at an even lower spatial resolution as they are based on hybridizing oligonucleotides to the sample and then selectively releasing bound oligonucleotides in a region-selective fashion followed by next-generation sequencing of the released oligonucleotides.

Spatial biology techniques based on combinatorial labeling have been developed, wherein combinations of dyes are used to encode the identity of a marker such as nanoreporters or nanostrings, which are used in Nanostring's CosMx imager, or HuluFiSH probes from PixelBiotech (Heidelberg, Germany) which are oligonucleotide-based compact combinatorial fluorescent labels, that can be used to label a larger number of markers, using a limited number of fluorescent dyes. In both of these cases, however, readout of the marker requires that only one labeled-marker is present in the readout volume at the same time. If multiple markers, such as one marking protein K and the other marking protein M are read out from the same readout volume at the same time, then reliable decoding is no longer possible. This poses a significant restriction as it basically means that reliable readout depends on sparse labeling of the sample. In practice this leads to stretching the readout over multiple rounds to avoid co-occurrence of two distinct markers in the same readout volume at the same time of readout (i.e. in the same round of an iterative staining, imaging, inactivation process). This is already a problem, when trying to analyse for example a large number such as of target mRNAs, but becomes a much more severe issue, when trying to analyse a comparable number of protein analytes as proteins are much more abundant in the cell and hence crowding is much more frequent.

### SUMMARY

Embodiments of the present invention provide a linker configured to bind to an affinity reagent. The linker includes at least one binding site configured to bind at least one dye. The linker further includes an oligonucleotide attachment portion. The oligonucleotide attachment portion is configured to bind to an oligonucleotide barcode of the affinity reagent.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 schematically shows two fluorescent dyes and commonly used affinity reagents;

FIG. 2 is a schematic drawing of spectrometers and dispersive optical elements;

FIG. 3 schematically illustrates how a set of dyes excitable with the same excitation light can be discerned using orthogonal dimensions;

FIG. 4 schematically summarizes different approaches for dye separation based on gating, unmixing, topographical approaches, phasor-based approaches which may or may not be combined with ML/DL/AI approaches to train dye separation classifiers;

FIG. 5 schematically shows a plurality of directly dye-conjugated markers grouped in sets;

FIG. 6A schematically shows a plurality of dyes yA+yB+yC . . . +yn grouped in n sets of dyes (set A to set n) excitable with different excitation lights;

FIG. 6B schematically illustrates the relationship between the plurality of dyes (YD), the plurality of combination of dyes (S1) and the plurality of affinity reagents (A=S2=T1*);

FIG. 6C schematically illustrates the relationship between the plurality of dyes (YD), the plurality of combination of dyes (S1) and the plurality of affinity reagents (A=S2=T1*);

FIG. 6D schematically illustrates the relationship between the plurality of dyes (YD), the plurality of combination of dyes (S1) and the plurality of affinity reagents (A=S2=T1*) and the plurality of markers (M);

FIGS. 6E to 6H further schematically illustrate the relationship between the plurality of dyes (YD), the plurality of combination of dyes (S1) and the plurality of affinity reagents (A=S2=T1*) and the plurality of markers (M);

FIG. 7A schematically shows different ways of using plurality of dyes (yA+yB+yC . . . +yn) to generate unique codes (set-based and binary combinatorial coding);

FIG. 7B schematically illustrates a plurality of affinity reagents (A=S2=T1*);

FIG. 7C schematically shows scheme for code readout using sequential excitation (set-based and binary combinatorial coding);

FIG. 8 schematically shows a bijective mapping or pair of combinations of dyes with unique oligonucleotide sequence barcodes;

FIG. 9 schematically illustrates a target molecule bound by a marker consisting of an affinity reagent and a reporter;

FIG. 10A schematically illustrates how multiple copies of the same dye species may be connected to a marker;

FIG. 10B schematically illustrates how information about the markers may be stored in a database and memory device;

FIG. 11 schematically shows an example of sequential readout of a marker wherein the affinity reagent is an antibody;

FIG. 12 schematically shows an example of sequential readout of a marker wherein the affinity reagent is an oligonucleotide probe;

FIG. 13 schematically shows the independence of the readout on spatial placing of dyes in or on the reporter for subdiffraction limit-sized reporters;

FIG. 14 is a bar chart illustrating the number of unique codes attainable with different n and yA+yB+yC . . . +yn attainable by using available dye and detector technology;

FIG. 15 is a schematic of a cell;

FIG. 16 is a schematic drawing illustrating the relative sizes of affinity reagents and dyes;

FIG. 17 is a schematic drawing illustrating the relative sizes of an antibody, a 10 nm quantum dot and the PSF of a 1.4 NA objective;

FIG. 18 is a flow chart describing an iterative staining process;

FIG. 19 is a schematic drawing of a cell in a well filled with hydrogel or in a hydrogel bead, wherein capture reagents are coupled to the hydrogel;

FIGS. 20A and 20B are schematic drawings of a cell in a well filled with hydrogel or in a hydrogel bead, wherein capture reagents are coupled to microbeads embedded into the hydrogel;

FIG. 21 is a schematic drawing of a cell in a well filled with hydrogel or in a hydrogel bead, wherein a plurality of spots is readout, decoded, and counted to derive a secretion profile of that cell;

FIG. 22 is flowchart of a spot detection and analysis pipeline;

FIG. 23 is a schematic drawing of a cell in well of a sample carrier and a readout device;

FIG. 24 is a schematic drawing of cells in hydrogel beads readout by a cytometer or imaging cytometer or microscope while flowing through a flow cell;

FIG. 25 is a schematic drawing of a bead-based assay readout with a cytometer, wherein capturing reagents covalently attached to microbeads capture target proteins, which are in turn marked by markers;

FIG. 26 is a schematic drawing of a bead-based assays to detect the presence of a target molecule or analyte as well as a bead-based assays to detect molecules interacting with a target molecule;

FIG. 27 is a schematic drawing of a cell-based assay readout with a cytometer, wherein markers are bound to molecules expressed at the cell surface;

FIG. 28 schematically illustrates the difference between mono-species readout volumes and multi-species readout volumes;

FIG. 29 schematically illustrates multi-species readout volumes including dyes excited with a first excitation light and the resulting first readout sequence;

FIG. 30 schematically illustrates a strategy for rendering the decoding of multi-species readout volumes in a different way based on dye blinking and localization microscopy;

FIG. 31 schematically illustrates the impact of different codes Ci or ciphers Xi on y and a for an example with yA=yB=yC=yD=yE=10 and n=5;

FIG. 32 schematically illustrates the impact of different examples of first readout sequences on κ;

FIG. 33A schematically illustrates the “plurality of combinations of dyes (S1)” containing all unique codes or combination of dyes and the first “set of combinations of dyes assigned to a marker” unsorted;

FIG. 33B schematically illustrates the “plurality of combinations of dyes (S1)” containing all unique codes or combination of dyes and the second “set of combination of dyes assigned to a marker” unsorted;

FIG. 33C schematically illustrates the “plurality of combinations of dyes (S1)” containing all unique codes or combination of dyes and the third “set of combination of dyes assigned to a marker” unsorted;

FIG. 34A schematically illustrates the “plurality of combinations of dyes (S1)” sorted into subsumable and non-subsumable under the first readout sequence;

FIG. 34B schematically illustrates the “plurality of combinations of dyes (S1)” sorted into subsumable and non-subsumable under the second readout sequence;

FIG. 34C schematically illustrates the “plurality of combinations of dyes (S1)” sorted into subsumable and non-subsumable under the third readout sequence;

FIG. 35A schematically illustrates an exemplary marker μ2015 in the first round;

FIG. 35B schematically illustrates an exemplary marker μ2015′ in the second round;

FIG. 35C schematically illustrates an exemplary marker μ2015″ in the third round;

FIG. 36A is a bar chart with an example probability of observing the same non-present marker for 2 to 5 iterations for κ=1000 in all rounds and ψ=100,000;

FIG. 36B is an example illustrating the impact of ψ and κ on the probability p of observing the same non-present marker for multiple iterations;

FIG. 37 is an iterative workflow for multi-spot primary qualitative decoding;

FIG. 38A schematically illustrates how intensity information is used for secondary qualitative and quantitative decoding;

FIG. 38B schematically illustrates how a first readout sequence can be intensity-thresholded reducing the number of combinations of dyes assigned to a marker and subsumable under the readout sequence;

FIG. 39 schematically illustrates a marker with a directly conjugated linker oligonucleotide and indirectly oligonucleotide-coupled dyes;

FIG. 40 schematically illustrates a way of assembling a combinatorial fluorescent code on a linker containing multiple dye-unspecific coupling or attachment sites;

FIG. 41 schematically illustrates a way of assembling a combinatorial fluorescent code on a linker containing multiple dye-specific coupling or attachment sites ensuring stoichiometric dye coupling;

FIG. 42 schematically illustrates a way of assembling a combinatorial fluorescent code on a micro-/nanobead containing multiple dye-unspecific coupling or attachment sites;

FIG. 43 schematically illustrates a way of enclosing a combination fluorescent code in a micro-/nanobead or micro-/nanocapsule;

FIG. 44 schematically illustrates a way of linking a combination of SMILES to an affinity reagent using a linker peptide, polymer, or oligonucleotide;

FIG. 45 schematically illustrates a way of linking a combination of SMILES to an affinity reagent using a nanostructure as an attachment platform;

FIG. 46 schematically shows how SMILES can be incorporated into a micro-/nanobead;

FIG. 47 schematically shows how SMILES can be incorporated into a micro-/nanocapsule;

FIG. 48A schematically shows oligonucleotide-, polymer/peptide-, nanoruler/DNA-origami-based nanostructure-linkers;

FIG. 48B schematically shows examples of a peptide-linker and an oligonucleotide-microbead-linker;

FIG. 49 schematically shows different options for coupling a linker to the affinity reagent;

FIG. 50A schematically shows the main and partially optional functional units of a readout device;

FIG. 50B schematically illustrates a microscope;

FIG. 51 shows 3 dyes with substantial spectral overlap, which can be unmixed by excitation spectral imaging. The diagram on top shows the excitation spectra of the three dyes, the diagram on bottom shows the emission spectra of the three dyes. The dyes shown are suitable for use with embodiments of the present invention;

FIG. 52 schematically shows a cyclical DNA exchange process;

FIGS. 53A-1, 53A-2, 53B, 53C schematically show respectively a readout volume and a readout trace;

FIG. 54 further schematically illustrates the assignment of two affinity reagents to a subset of combinatorial labels comprising a subset of combinations of dyes;

FIGS. 55, 55A, 55B and 55C show a schematic illustration of how readout traces, progressive decoding, statistical analysis, presence calling, mathematical input/output models and a priori information sources may be related to each other;

FIG. 56 schematically illustrates short and long residence times of unbound and bound reporters;

FIG. 57 schematically illustrates a strategy that enables “barcode cycling” and thereby intra-round code swapping in a dynamic or repeated fashion;

FIG. 58 illustrates association and dissociation of guest molecule-modified oligonucleotides;

FIG. 59 illustrates barcode cycling using barcoded antibodies as affinity reagents;

FIG. 60 illustrates barcode cycling using oligonucleotide probes as affinity reagents; and

FIG. 61 illustrates an alternative strategy that enables barcode cycling and thereby intra-round code swapping in a dynamic or repeated fashion.

### DETAILED DESCRIPTION

Embodiments of the present invention allow very high number of markers to be readout by means of a fluorescence-based optical readout, which may be based on a continuous data readout stream or discrete readout (digital or analog) and may be based on point-detectors, line-detectors or area detectors such as cameras or hyperspectral cameras for example. The method is therefore widely applicable in life sciences, diagnostics, environmental sciences, and healthcare and quality control and can be combined with a wide array of optical readouts. These include but are not limited to cytometers, plate readers, microscopes, imaging systems.

Embodiments of the present invention can achieve marker discrimination capability and coverage rates attainable presently with next-generation sequencing-based readouts on the basis of an optical fluorescence-based readout and can be implemented on commercially available fluorescence imaging systems, such as for example the STELLARIS 8 confocal microscope platform (from Leica Microsystems).

Embodiments of the present invention are based on “looking at” microscopy as an encoding/decoding problem rather than a problem of registering spatially located intensities in an image, which is essentially a matrix of intensity values. While the method according to embodiments of the present invention is compatible with image-based readouts, the “images” generated by the method and device according to embodiments of the present invention should be regarded as probabilistic mathematical models of the reality of the sample under investigation, in which the presence of a target molecule is detected or called (presence calling) based upon the decision of the user to accept its presence based on a measure of statistical confidence and a certain level of statistical confidence in the presence of the respective target molecule or analyte in the readout volume.

Making the step of accepting the presence of a certain marker, a certain affinity reagent, and a certain target molecule based on a measure of statistical confidence and/or a certain level of statistical confidence (i.e. presence calling) generates a mathematical truth. This is an important aspect of the present invention as this also implies that following to presence calling one is operating in the axiomatic domain of mathematics which is inherently free of influences that complicate measurements in the non-mathematical domain, i.e. the physical, chemical, biological domain. This fact therefore has important implications and also indicates that this method enables a completely new microscopic modality.

The statistical methods to provide a measure of statistical confidence on a per marker-basis or per target-molecule basis, may well be a combined measure and may in many ways be similar or identical to methods, which are used in transcriptomics and genomics, where enrichment scores and p values are commonly used.

Embodiments of the present invention use combinatorial fluorescent labels, which comprise unique combinations of dyes, which are essentially codes. The term “code” and the term “combinatorial fluorescent label” and the term “combinatorial label” are therefore used interchangeably in the present document. As described above other methods using such combinatorial labels, such as nanoreporters (Nanostrings) described in U.S. Pat. No. 9,376,712 B2, U.S. Pat. No. 8,519,115 B2, exist in the prior art. Likewise methods exist in the prior art that encode the identity of a marker in a certain combination of dyes or a certain sequence of 0 and 1 over multiple cycles of an iterative staining, imaging, inactivation process. This latter readout method has been referred to as readout in a “temporally sequential manner” as the code is not read in a single cycle, but read out over multiple cycles, which is different from what has been referred to as “spatial readout”, meaning the reading of the code in a single cycle.

Examples for technologies with readout in “temporally sequential manner” are MERFISH, SeqFISH or the method disclosed in U.S. Pat. No. 10,227,639 B2, whereas examples for technologies with “spatial readout” are nanoreporters (Nanostrings) described in U.S. Pat. No. 9,376,712 B2 or HuluFiSH (PixelBiotech, Heidelberg).

From these conceptual elements of the prior art as explained in the application PCT/EP2021/073819, the complete content thereof being incorporated herein by reference, and/or as explained in the application PCT/EP2021/066645, the complete content thereof being incorporated herein by reference, involved:
a plurality of unique combinatorial fluorescent labels, which are physical structures that comprise a backbone onto which a unique combination of dyes can be assembled either before, during, or after introduction into the sample;
a linker or linking element or linking structure that mediates the connection between the combinatorial label and an affinity reagent;
a mapping wherein each affinity reagent is assigned to one unique combinatorial label for each round.

The core idea as explained in the applications PCT/EP2021/073819 and PCT/EP2021/066645 was the realization that a major limitation of the prior art, i.e. the inability to reliably read the identity of multiple markers present in the same readout volume at the same time, can be overcome by introducing “code swapping”, i.e. a change in the mapping between affinity reagents and combinatorial labels, from the nth round to the n+1th round. Using “code swapping” and an iterative process or cyclical process of staining, imaging, and inactivation, readout volumes containing multiple markers, for example labeled antibodies against 5 different proteins, at the same time can be reliably decoded on certain level of statistical confidence over a limited number of rounds. The decoding strength, i.e. how quickly the confidence in the decoding result rises, thereby depends on the distance of codes in the code space, or in other words on how different the overlapping combinatorial labels are with respect to their dye composition, or in other words it depends on the number of dyes from which combinations can be generated as well as to the selection of combinations that are used from the plurality of all possible combinations. While the methods disclosed in the applications PCT/EP2021/073819 and PCT/EP2021/066645 are extremely powerful, however, it still might be dependent on an iterative process or cyclical process of staining, imaging, inactivation.

Embodiments of the present invention improve upon this method and to remove the need for using an iterative process.

The methods described in the applications PCT/EP2021/073819 and PCT/EP2021/066645 fundamentally might rely on labeling the sample with markers comprising combinatorial labels, which might be swapped from one round to the next (“code swapping”), and the acceptance of the presence of a marker in the readout volume based on a statistical analysis of a measure of statistical confidence (“presence calling”).

An important aspect of the present invention is the realization that the “code swapping” does not necessitate a cyclical process of staining, imaging, inactivation. Using oligonucleotide-barcoded affinity reagents, wherein each affinity reagent comprises a unique oligonucleotide-barcode, and combinatorial labels that are configured to bind to said oligonucleotide-barcodes, it is possible to perform “code swapping” without inactivation and restaining. This is in particular possible, when the following conditions are fulfilled:
(A) a subset of unique combinatorial labels from the plurality of combinatorial labels is assigned to an affinity reagent from the plurality of affinity reagents;
(B) said subset of unique combinatorial labels is not assigned to more than one affinity reagent; and
(C) connection between the oligonucleotide-barcoded affinity reagent and the unique combinatorial labels is based on transient or reversible hybridization and/or dynamic association/dissociation or reversible association/dissociation.

In this case when a certain readout volume is observed containing a marker comprising an affinity reagent, for example an antibody against protein X, labeled with a first combinatorial label k from its assigned set of unique combinatorial labels, then this label k will dissociate after some time and the oligonucleotide-barcode of the antibody is free to bind another label from the set such as label f.

We refer to this as intra-round “code swapping” and to the enabling processes as “barcode cycling”. This barcode cycling can be performed in various ways that either enable transient binding or reversible binding between the oligonucleotide-barcode and the respective oligonucleotide portion on the combinatorial label. The various ways, hereinafter referred to as (1) to (7), but are not limited to:
(1) DNA exchange imaging, which is closely related to DNA-PAINT and Exchange-PAINT, which can be used to enable dynamic or repeated intra-round code swapping.
(2) DNA-PAINT, wherein the oligonucleotide portion of the label and the respective oligonucleotide-barcode of the respective affinity reagent are configured to bind to each other transiently allowing “dynamic intra-round code swapping”. This may be achieved for example by using complementary sequence stretches of about 9-13 nt in length.
(3) Exchange-PAINT, which is a combination of DNA-Paint and exchange imaging.
(4) Strand displacement, wherein the oligonucleotide portion of the label that hybridizes to the oligonucleotide-barcodes of the respective affinity reagent binds suboptimally due to mismatches and can be displaced by introducing a free oligonucleotide that binds perfectly (no mismatches) to the oligonucleotide portion of the label that exhibits imperfect complementarity to the oligonucleotide-barcode of the respective affinity reagent.
(5) Photoswitchable NCDA, which is a molecular glue for DNA that consists of “two guanine-recognizing naphthyridine moieties connected to a photochromic azobenzene unit, which undergoes cis/trans isomerizations by photoirradiation”. NCDA can be used to enable the hybridization or reverse hybrization of short GG mismatch containing oligonucleotides. Switching is performed using 360 nm and 430 nm light. NCDA mediated reversible binding may also allow the repeated binding/unbinding of distinct combinatorial labels to the same barcoded affinity reagent without necessitating an iterative staining process. The use of NCDA as molecular glue for DNA is described in Dohno et al. 2007 (Journal of the American Chemical Society 2007 129 (39), 11898-11899).
(6) Controllable DNA hybridization by host-guest complexation mediated-ligand invasion further can be used as a strategy to enable reversible binding/unbinding or transient binding. Using cucurbit[7]uril the authors of Xiao, L., Wang, L L., Wu, C Q. et al. Controllable DNA hybridization by host-guest complexation-mediated ligand invasion. Nat Commun 13, 5936 (2022) achieved ligand-controlled DNA hybridization.
(7) Temperature cycling, which allows also longer predetermined sequences or attachment sites to be used for intra-round “code swapping”. Depending on buffer conditions, and the G/C content of the predetermined sequences or attachment sites melting temperatures maybe in the range of 30-72° C., preferably 50-72° C.

### Terms

In the sense of this document the following terms are used in the following way:

“Sample”: In the sense of this document “sample” refers to a biological sample which may also be named a biological specimen including, for example blood, serum, plasma, tissue, bodily fluids (e.g. lymph, saliva, semen, interstitial fluid, cerebrospinal fluid), feces, solid biopsy, liquid biopsy, explants, whole embryos (e.g. zebrafish, Drosophila), entire model organisms (e.g. zebrafish larvae, Drosophila embryos, C. elegans), cells (e.g. prokaryotes, eukaryotes, archea), multicellular organisms (e.g. Volvox), suspension cell cultures, monolayer cell cultures, 3D cell cultures (e.g. spheroids, tumoroids, organoids derived from various organs such as intestine, brain, heart, liver, etc.), a lysate of any of the aforementioned, a virus. In the sense of this document “sample” further refers to a volume surrounding a biological sample. Such as in assays, where secreted proteins like growth factors, extracellular matrix constituents are being studied the extracellular environment surrounding a cell up to a certain assay-dependent distance, is also referred to as the “sample”. Specifically, affinity reagents brought into this surrounding volume are referred to in the sense of this document as being “introduced into the sample”.

“Affinity reagent”: In the sense of this document the term “affinity reagent” may in particular be an antibody, a single-domain antibody (also known as nanobody), a combination of at least two single-domain antibodies, an aptamer, an oligonucleotide, a morpholino, a PNA complementary to a predetermined RNA, DNA target sequence, a ligand (e.g. a drug or a drug-like molecule), or a toxin, e.g. Phalloidin a toxin that binds to an actin filament. In the sense of this document an affinity reagent is configured to bind a target molecule or to an analyte with a certain affinity and specificity such that it can be said that the affinity reagent is substantially specific to the target molecule or predetermined target structure. In the sense of this document “plurality of affinity reagents” (S2) contains the affinity reagents (a1, a2, a3, . . . an), which are configured to specifically bind to a predetermined target structure within the biological sample or to a predetermined chemical compound or to a predetermined chemical element or to an analyte. At least some of the affinity reagents from the plurality of affinity reagents (A) are “introduced to the sample” such that the affinity reagents can attach to the respective predetermined target structure within the sample. In this context and in the sense of this document and as described above “introduced to the sample” may refer to being physically introduced into the volume of the sample or into a volume surrounding and assigned to the sample. An example of the latter case may be assays for secreted molecules for instance, which are best assessed in the extracellular space where they might be outside of the sample, but within a certain spatial context or vicinity of the sample. In the case of nucleic acid targets or analytes such as DNA targets or RNA targets an affinity reagent like an oligonucleotide-probe, may refer to a set of distinct affinity reagents, like a set of oligonucleotide-probes binding to the same analyte, e.g. the same gene, or the same locus, or the same mRNA target, at different overlapping or non-overlapping positions or sequence stretches, in the sense of this document. Code swapping in this case, refers to labeling the same analyte nucleic acid with different combinations of dyes attached to distinct affinity reagents, e.g. oligonucleotide probes binding to different sites on the same nucleic acid target, e.g. mRNA.

“Predetermined target structure”: In the sense of this document “predetermined target structure” refers to a target molecule or a target structure or to an analyte, which may for example be a protein (e.g. a certain protein), an RNA sequence (e.g. the mRNA of a certain gene), a peptide (e.g. somatostatin), a DNA sequence (e.g. the a genetic locus or element), a metabolite (e.g. lactic acid), a hormone (e.g. estradiol), a neurotransmitter (e.g. dopamine), a vitamin (e.g. cobalamine), a micronutrient (e.g. biotin), a metal ion (e.g. metal and heavy metal ions like Cd(II), Co(II), Pb(II), Hg(II), U(VI)).

“Dye”: In the sense of this document the terms “fluorescent dye”, “fluorophore”, “fluorochrome”, “dye” are used interchangeably to denote a fluorescent chemical compound or structure and can be in particular one of the following: a fluorescent organic dye, a fluorescent quantum dot, a fluorescent dyad, a fluorescent carbon dot, graphene quantum dot or other carbon-based fluorescent nanostructure, a fluorescent protein, a fluorescent DNA origami-based nanostructure. From the organic fluorescent dyes in particular derivatives of the following are meant by the term “fluorescent dye”: xanthene (e.g. fluorescein, rhodamine, Oregon green, Texas), cyanine (e.g. cyanine, indocarbocyanine, oxacarbocyanine, thiacarbocyanine, merocyanine), derivatives, squaraine rotaxane derivatives, naphthalene, coumarin, oxadiazole, anthracene (anthraquinones, DRAQ5, DRAQ7, CyTRAK Orange), pyrene (cascade blue), oxazine (Nile red, Nile blue, cresyl violet, oxazine 170), acridine (proflavine, acridine orange, acridine yellow), arylmethine (auramine, crystal violet, malachite green), tetrapyrrole (porphin, phthalocyanine, bilirubin), dipyrromethene (BODIPY, aza-BODIPY), a phosphorescent dye, or a luminescent dye. The following trademark groups designated commercially available fluorescent dyes, which may include dyes belonging to different chemical families CF dye (Biotium), DRAQ and CyTRAK probes (BioStatus), BODIPY (Invitrogen), EverFluor (Setareh Biotech), Alexa Fluor (Invitrogen), Bella Fluore (Setareh Biotech), DyLight Fluor (Thermo Scientific), Atto and Tracy (Sigma-Aldrich), FluoProbes (Interchim), Abberior Dyes (Abberior Dyes), Dy and MegaStokes Dyes (Dyomics), Sulfo Cy dyes (Cyandye), HiLyte Fluor (AnaSpec), Seta, SeTau and Square Dyes (SETA BioMedicals), Quasar and Cal Fluor dyes (Biosearch Technologies), SureLight Dyes (Columbia Biosciences), Vio Dyes (Milteny Biotec) [list modified from: https://en.wikipedia.org/wiki/Fluorophore]. From the group of fluorescent proteins in particular the members of the green fluorescent protein (GFP) family including GFP and GFP-like proteins (e.g DsRed, TagRFP) and their (monomerized) derivatives (e.g., EBFP, ECFP, EYFP, Cerulaen, mTurquoise2, YFP, EYFP, mCitrine, Venus, YPet, Superfolder GFP, mCherry, mPlum) are meant by the term “fluorescent dye” in the sense of this document. Further from the group of fluorescent proteins the term “fluorescent dye” in the sense of this document may include fluorescent proteins, whose absorbance or emission characteristics change upon binding of ligand such as BFPms1 or in response to changes in the environment such as redox-sensitive roGFP or pH-sensitive variants. Further from the group of fluorescent proteins the term “fluorescent dye” in the sense of this document may include derivative of cyanobacterial phycobiliprotein small ultra red fluorescent protein smURFP as well as fluorescent protein nanoparticles that can be derived from smURFP. An overview of fluorescent proteins can be found in Rodriguez et al. 2017 in Trends Biochem Sci. 2017 February; 42(2): 111-129. The term “fluorescent dye” in the sense of this document may further refer to a fluorescent quantum dot. The term “fluorescent dye” in the sense of this document may further refer to fluorescent carbon dot, a fluorescent graphene quantum dot, a fluorescent carbon-based nanostructure as described in Yan et al. 2019 in Microchimica Acta (2019) 186: 583 and Iravani and Varma 2020 in Environ Chem Lett. 2020 Mar. 10: 1-25. The term “fluorescent dye” in the sense of this document may further refer to a fluorescent polymer dot (Pdot) or nanodiamond. The term “fluorescent dye” in the sense of this document may further refer to a fluorescent dyad, such as a dyad of a perylene antenna and a triangelium emitter as described in Kacenauskaite et al. 2021 J. Am. Chem. Soc. 2021, 143, 1377-1385.

The term “fluorescent dye” in the sense of this document may further refer to an organic dye, a dyad, a quantum dot, a polymer dot, a graphene dot, a carbon-based nanostructure, a DNA origami-based nanostructure, a nanoruler, a polymer bead with incorporated dyes, a fluorescent protein, an inorganic fluorescent dye, a SMILE, or a microcapsule filled with any of the aforementioned.

The term “fluorescent dye” in the sense of this document may further refer to a FRET-pair having at least one fluorescent dye as FRET donor and at least one fluorescent dyes as a FRET acceptor, or a FRET-triple, which is used to generate a three component Forster resonance energy transfer. In particular, the FRET-pair or FRET-triplet is connected by a complementary linker or by a linking element.

The term “fluorescent dye” in the sense of this document may further refer to a FRET n-tupel of physically connected dyes.

“Plurality of combinations of dyes” (S1): In the sense of this document the term “Plurality of combinations of dyes” (S1) refers to the plurality of combinations of dyes for which each combination of dyes (s1, s2, s3, . . . sn) is unique within the plurality of combinations of dyes (S1), each combination of dyes (s1, s2, s3, . . . sn) comprises at least two different dyes (|s|>=2); wherein the plurality of combinations of dyes (S1) is composed such that each dye (y1, y2, y3, . . . y□) in the plurality of combinations of dyes (S1) can be readout by a readout device; wherein dyes can be separated by a readout device into channels; each channel corresponding to one of the dyes (y1, y2, y3, . . . y□).

“Marker”: In the sense of this document “marker” is used to denote both a single molecule used as marker and a collection of identical molecules used as marker. A “marker” in the sense of this document is the combination of an affinity reagent configured to attach to a predetermined structure also referred to as a target molecule or an analyte and/or a “reporter”. As such the “marker” is the virtual assignment or mapping of an affinity reagent to a particular combination of dyes (virtual marker) and the physical assembly of an affinity reagent with the combination of dyes (physical marker). In the sense of this document, the physical assembly of an affinity reagent with the combination of dyes may occur before, during, or after the introduction of the respective affinity reagent into the sample. For example, when oligonucleotide sequence barcoded antibodies are used as affinity reagents, they may be brought into a sample and allowed to attach to their predetermined target structure, e.g. by physically attaching the unique combination of dyes (si) to the assigned affinity reagent (ai), before or after introducing at least some affinity reagents from the plurality of affinity reagents (A) to the sample or to the chemical compound or to the chemical element, or before a generation of a readout from emission light emitted by excited dyes. In an iterative staining-imaging-dye deactivation process an affinity reagent bound to a predetermined target structure may be cyclically connected to a sequence of different combinations of dyes, as in a first combination of dyes in a first iteration and a second combination of dyes in a second iteration, a strategy to which we refer as “Primary qualitative iterative multi-species readout volume decoding by reassigning codes in between iterations (“code swapping”)”. In other words, one or more of the markers in the sample will change between iterations.

“Reporter”, “Combinatorial Label”: In the sense of this document “reporter” is used to denote both a single molecule/structure used as reporter and a collection of identical molecules/structures used as reporter. A “reporter” in the sense of this document is the combination of a unique “combination of dyes” and “linker”, configured to connect the combination of “dyes” with the “affinity reagent”.

“Code”, “Code swapping”: In the sense of this document a “reporter” or “combinatorial label” comprises a unique “combinations of dyes”, which resembles a “code” that encodes the identity of a particular affinity reagent and thereby the identity of a specific marker (marker=affinity reagent+combinatorial label) and thereby the identity of a certain analyte such as the mRNA of gene X or the protein X. In the sense of this document a “subset of unique combinations of dyes” and therewith a “subset of combinatorial labels” is assigned to a particular affinity reagent forming a “subset of markers”, wherein one affinity reagent is assigned to a plurality of “combinations of dyes” or “codes”. The respective affinity reagent is then allowed to contact its analyte in a biological sample and at least one of the combinatorial labels of the “subset of combinatorial labels” is allowed to associate with said affinity reagent forming a first marker of the “subset of markers”. The fluorescent dyes of the attached “combinatorial label” are readout, the “combinatorial label” is allowed to detach from the affinity reagent and a second “combinatorial label” from the “subset of combinatorial labels” assigned to said affinity reagent is allowed to associate with it. This process, which leads to change of association between the affinity and its assigned combinatorial labels is referred to as “code swapping”. In a previous patent application this “code swapping” was performed in between rounds of a cyclical staining, imaging, inactivation process. In the present document this “code swapping” is performed “intra-round”, i.e. does not necessitate a cyclical staining, imaging, inactivation process and basically happens in a partial first round (which consist only of staining and imaging). The “intra-round code swapping” may be performed using transient binding between “combinatorial labels” and assigned affinity reagents and their stochastic association, which is referred to in this document as “dynamic intra-round code-swapping”, or it may be brought about by a switching between stable binding between “combinatorial labels” and assigned affinity reagents and release, which may be implemented in various ways detailed in the description. This latter mode is referred to in this document “repeated intra-round code-swapping”.

“Linker”: In the sense of this document the linker denotes a unipartite chemical structure (e.g. a monomeric molecule or a polymer) or multipartite assembly of chemical structures linking a combination of fluorescent dyes to an affinity reagent. A linker might be directly or covalently coupled to the dyes and to the affinity reagent or indirectly through for example affinity tag-affinity ligand combination such as streptavidin-biotin interaction or a hapten or an oligonucleotide for example. In the case of covalent coupling this may be a site-selective coupling. Commonly used coupling chemistries such NHS-, maleimide, azide-alkine and a range of further so called click chemistries may be used to couple the linker to the affinity reagent and/or the linker to a dye. A linker may in particular comprise an oligonucleotide (e.g. DNA, RNA, LNA, PNA, morpholino, other artificial oligonucleotide), a peptide, a DNA-origami-based structure such as for example a nanoruler, a micro-/nanobead, a polymer, a micro-/nanocapsule, a micro-/nanocrystal, a carbontube, a carbon-based nanostructure (e.g. a graphene).

A linker may in particular comprise an oligonucleotide and another element of the group mentioned before, such as comprise an oligonucleotide and a peptide.

“Readout device”: In the sense of this document “readout device” refers to a device used to perform fluorescence multicolour reading or imaging. A readout device typically includes at least one excitation light source, a detection system including at least one detection channel and may as well contain filters and/or dispersive optical elements such as prisms and/or gratings to route excitation light to the sample and/or to route emission light from the sample onto to a detector or onto an appropriate area of the detector. The detection system in the sense of this document may comprise several detection channels, may be a spectral detector detecting multiple bands of the spectrum in parallel, or a hyperspectral detector detecting a contiguous part of the spectrum. The detection system comprises at least one detector, which may be a point-detector (e.g. a photomultiplier, an avalanche diode, a hybrid detector), an array-detector, a camera, hyperspectral camera. The detection system may record intensities per channel as is typically the case in cytometers or may be an imaging detection system that records images as in the case of plate readers or microscopes. A readout device with one detector channel, such as a camera or a photomultiplier, may generate readouts with multiple detection channels using, for example, different excitation and emission bands. Readout devices allow a certain number of dyes to be analysed from a given biological sample in a given run. A “run” may refer to an “iteration” or “round”, i.e. the production of at least one readout for a given set of combinations of dyes and a given mapping of affinity reagents to combinations of dyes, wherein the affinity reagents are attached to the analytes. This number typically depends on the number of detection channels, n, the readout device is configured to provide, i.e. is able to spectrally resolve. In the case of microscopes the number of detection channels is typically 4-5 in the case of camera-based widefield detections (e.g. widefield epifluorescence microscopes, spinning disk microscopes, light sheet fluorescence microscopes), 5-12 detection channels in the case of microscopes with spectral detection concepts that typically rely on excitation or emission fingerprinting and (spectral/linear) unmixing. Hyperspectral imaging instead, which can differentiate a high number of dyes by providing very fine spectral resolution over a wide and contiguous spectral range is not yet widely deployed in microscopy. In addition to spectral properties also the lifetime of fluorescent dyes can be used to discern multiple dye species and differentiate them from autofluorescence effectively increasing the number of detection channels, y, which might correspond also to the maximum number of dyes in a set (i.e. excitable by the same excitation light) that can be reliably separated.

“Oligonucleotide”: in the sense of this document refers to DNA, RNA, peptide nucleic acid, morpholino or locked nucleic acid, glycol nucleic acid, threose nucleic acid, hexitol nucleic acid or another form of artificial nucleic acid.

“Spot”: in the sense of this document refers to a volume in the sample or region surrounding the sample, which is being readout. The size and shape of spots is dictated by the effective point spread function of the imaging system used to acquire the data.

“Point spread function”:—In the sense of this document the term “point spread function” is used to denote the main maximum of the point spread function and unless otherwise denoted the term refers to the effective point spread function (PSF) of the imaging system, which is generally elliptical, i.e. the lateral resolution is better than the axial resolution, but may approach an almost spherical shape as more views are acquired from preferably equidistant angles.

“Readout”: In the sense of this document the term “readout” refers to an image-based readout, which may be acquired on a microscope like a point-scanning confocal or a camera-based/widefield imaging system such as a spinning disk microscope, a light sheet fluorescence microscope, a light field microscope, a stereomicroscope. Further the term “readout” refers to non-image based readouts such as in a cytometer or a flow-through based readout device with at least one point detector or a line detector. A readout may consist of a discrete readout, such as a single acquisition of an emission spectrum or image stack, a readout may be a readout data stream, such as in a point-scanning confocal or cytometer, which is substantially continuous. Further a readout may be a sequence of images for example a spectral or hyperspectral image stack, wherein in each image fluorescence emission of different wavelength bands is recorded.

“Readout volume”: In the sense of this document the term “readout volume” refers to the volume which is effectively detected by an optical system such as a microscope or a cytometer at a given moment in time. For systems with a continuous data stream, the “readout volume” is determined by a clock like a “pixel clock”, which divides a continuous data stream into chunks that are then assigned to a certain time point or spatial location. The readout volume might depend on the effective point spread function of the imaging system, e.g. an effective point spread function might define or confine the maximum extent of a readout volume.

“Readout sequence”: In the sense of this document the term “readout sequence” is used refer to a readout of a “readout volume” that readouts all dyes (y1, y2, y3, . . . y□) in the plurality of dyes YD from which the combinations of dyes in the plurality of combinations of dyes (S1) are composed at least one time, i.e. all dyes (y1, y2, y3, . . . y□) in the plurality of dyes YD are excited at least one time and the emitted fluorescence light is detected and separated by the readout device into channels, each channel corresponding to one of the dyes (y1, y2, y3, . . . y□). Such that after obtaining the readout sequence the presence or absence of a dye from the readout volume can be assessed qualitatively and/or quantitatively, wherein qualitatively refers to calling a dye present in the readout volume, when the intensity in the corresponding channel is above a certain user-defined threshold, wherein quantitatively refers to calling a dye present in the readout volume and assigning a relative intensity value or absolute number of molecules to it. The threshold may be a fixed threshold, a fixed channel-specific threshold, or a dynamically adjusted threshold. The threshold may be a combination of thresholds such as an intensity threshold and a statistical confidence in the dye separation result. The decision to call a dye present (presence calling) may be made dependent on passing a combination of multiple thresholds.

“Readout trace”: In the sense of this document the term “readout trace” refers to a times series acquisition of a “readout sequence”. In other words it is used to refer to a readout of a “readout volume” that readouts all dyes (y1, y2, y3, . . . y□) in the plurality of dyes YD from which the combinations of dyes in the plurality of combinations of dyes (S1) are composed at least two times. The difference to acquiring multiple “readout sequences” in a cyclical process to acquiring a “readout trace” is that a “readout trace” is not interrupted by pauses for inactivation of labels, washing and restaining. In a preferred embodiment, a biological sample is analysed by performing tile scans and recording short time series at each tile. Such “readout traces” may be acquired with intervals in the ms-min range or as a continuous acquisition (streaming) of particular position (tile). A “readout trace” may also be performed of a given volume by acquiring time series at a certain XY position while moving the sample relative to the readout in Z. In a particularly preferred embodiment of the invention a readout is used that is comprises an object plane orientation, which is at an angle, to the (optical) surface of a sample carrier (e.g. microscope slide, imaging well chamber, well plate bottom). This may in particular be a SCAPE, OPM, microscope or a readout device as it is described in the PCT-application with the application number PCT/EP2022/077378, the entire content thereof being incorporated by reference herein.

In embodiments of the present invention “readout sequences” are therefore acquired as times series, which are continuous at each position of the imaging area of the sample. In the applications PCT/EP2021/073819 and PCT/EP2021/066645 “readout sequences” are acquired in cycles or iteratively as “code swapping” depends on inactivation of bound combinatorial labels from round n, washing out the unbound or inactivated combinatorial labels from round n before a relabeling with at least some of the combinatorial labels that have been reassigned to at least some of the affinity reagents in round n+1. This may be referred to as inter-round “code swapping”, because codes are reassigned from one round to the next, whereas embodiments of the present invention remove the necessity to perform a cyclical process and “code swapping” is performed “intra-round”
either dynamically (“dynamic intra-round code swapping”), wherein a plurality of distinct combinatorial labels assigned to a particular affinity reagent are allowed to dynamically associate and dissociate with the oligonucleotide-barcode of said affinity reagent; or
repeatedly (“repeated intra-round code swapping”), wherein a plurality of distinct combinatorial labels assigned to a particular affinity reagent are allowed to repeatedly associate and dissociate with the oligonucleotide-barcode of said affinity reagent.

“Dynamic intra-round code swapping” can be performed by using DNA exchange imaging technology, controllable DNA hybridization strategies as well as simply by raising the temperature close to the melting temperature of the oligonucleotide-barcode-attachment portion of the linker interaction, where the association constant will largely depend on the length of the complementary stretch, number of mismatches (if any are introduced) and the ionic strength of the buffer. It is important to note that raising the temperature to a close to the melting point can be performed with oligonucleotide based linker or backbone structures of combinatorial labels, if they have been stabilized before using a suitable strategy such as for example ligation, or UV-mediated cross-linking, or chemical cross-linking. Further, “Dynamic intra-round code swapping” controllable DNA hybridization as described by Xiao et al. 2022 (Xiao, L., Wang, L L., Wu, C Q. et al., Controllable DNA hybridization by host-guest complexation-mediated ligand invasion, Nat Commun 13, 5936 (2022)) is achieved by keeping the ligand at a suitable concentration. In “Dynamic intra-round code swapping” different combinatorial labels assigned to a particular affinity reagent associate and dissociate randomly with the respective oligonucleotide-barcode.

“Repeated intra-round code swapping” can be performed using temperature cycling (melting-reannealing), controllable DNA hybridization as described by Xiao et al. 2022 (Xiao, L., Wang, L L., Wu, C Q. et al., Controllable DNA hybridization by host-guest complexation-mediated ligand invasion by changing the ligand concentration repeatedly, Nat Commun 13, 5936 (2022)), or by using photoswitchable NCDA as described by Dohno et al. 2007 (Journal of the American Chemical Society 2007 129 (39), 11898-11899).

Both (“dynamic intra-round code swapping”) and (“repeated intra-round code swapping”) may either be performed by introduction all necessary reagents to the sample before the acquisition of “readout traces” or by applying at least some of the reagents or modifying at least some of the physical parameters such as light illumination with UV, 360 nm, 430 nm light for example or temperature during the acquisition of the “readout traces”.

Preferably, a readout sequence results from exciting the sample with a first excitation light A, detecting the emitted fluorescence, and assigning it to yA channels corresponding to DyeA1, DyeA2, DyeA3 . . . DyeAyn, a second excitation light B, detecting the emitted fluorescence, and assigning it to yB channels corresponding to DyeB1, DyeB2, DyeB3 . . . DyeByn, and repeating the process until Dyen1, Dyen2, Dyen3, . . . Dyenyn (i.e. the entire plurality of dyes (YD) with y□ members) have been readout at least once.

A “code” in the sense of this document is defined as follows: S and T are two finite sets, with S being named the “source alphabet” and T being named the “target alphabet”. A Code

$$C : S \to {T}^{*}$$

is a total function or algorithm that uniquely represents an element from S as a sequence of symbols over T. The extension C′ of C, is a homomorphism of S* into T*, which naturally maps each sequence of source symbols to a sequence of target symbols. In the language used in computer science a code is generally referred to as an algorithm and a sequence of symbols as an encoded string (modified from: Code. (n.d.) In Wikipedia. Retrieved Jun. 17, 2021 from https://en.wikipedia.org/wiki/Code). In the sense of this document the finite set S1 is also named the “plurality of combination of dyes”, and T1* is the finite set of strings over T1 and corresponds to the “plurality of affinity reagents”, which may also be named A or S2.

Alternatively, or in addition to the encoding/decoding of combinations of dyes users may encrypt/decrypt combinations of dyes using a cipher X

Two different cases a and b are being discerned and can be regarded as different directionalities of encoding/encryption.

$$\begin{matrix}{S}_{1} {\to }^{{C}_{α 1}} {T}_{c 1} * = A \\ or \\ {S}_{1} {\to }^{{X}_{α 1}} {T}_{x 1} * = A\end{matrix} \begin{matrix}A = {S}_{2} {\to }^{{C}_{β 2}} {T}_{c 1} * \\ or \\ A = {S}_{2} {\to }^{{X}_{β 2}} {T}_{X 1} *\end{matrix}$$

The method disclosed in this document is compatible with both cases □ and □. As well as with cases in which multiple codes C1, C2, C3, . . . Cn and/or ciphers X1, X2, X3, . . . Xn are being used so as long as they are total functions and as long as the resulting mapping is injective or bijective. In both of these cases the codes C1, C2, C3, . . . Cn and/or ciphers X1, X2, X3, . . . Xn are functions that can be inverted, i.e. decoded. In a particularly preferred embodiment of the invention, a bijective mapping (encoding or encryption) is used, which means that there is a one-to-one correspondence between an element (ai) of the plurality of affinity reagents (S2) also named (A) or (T1*) and an element (si) of the plurality of combination of dyes (S1). This allows particularly easy decoding of the combination of dyes (sl to sk) contained in the readout volume and thereby the identification of their associated affinity reagents (al to ak) based on a readout sequence, that assesses the presence of all dyes (y1, y2, y3, . . . y□) in the readout volume qualitatively (presence calling, e.g. “yes”=“1”, “no”=0) and/or quantitatively (presence calling with relative or absolute quantitation). The microscopic examination of the readout volume as described in this document can be regarded as encoding/decoding problem, which is solved by labeling target molecules with affinity reagents that are (dynamically) linked to, and associated with, combinations of dyes, which encode those target molecules in the readout volume labeled in this way. Retrieving the identity of the target molecules which have a one-to-one mapping (bijective association) with the affinity reagents from the plurality of the affinity reagents is thus a decoding problem. It is important to state, that if the presence of a certain dye from the plurality of dyes has been accepted in a readout sequence based on a certain degree of statistical confidence, then that presence becomes a mathematical truth. The decoding of combinations of dyes (sl to sk) is possible, when a readout sequence is observed that does not allow all possible combinations of dyes (s1, s2, s3, . . . sn) in the plurality of combinations of dyes (S1) to be subsumed under it. In other words, if a readout sequence is observed under which all possible combinations of dyes (s1, s2, s3, . . . sn) in the plurality of combinations of dyes (S1) can be subsumed, then one does not gain knowledge about the contents of the readout volume. This would be the case if a readout sequence would indicate that all dyes (y1, y2, y3, . . . y□) from the plurality of dyes (PD) are called “present” in the readout volume. This case is, however, unlikely even for cases in which large numbers of affinity reagents are being used, as the cardinality of the plurality of combinations of dyes (S1) grows exponentially, while the number of available affinity reagents is limited to the number of targets molecules of interest. For example the entire human genome contains about 20,000 coding genes, so even if one would use 20,000 affinity reagents in the plurality of affinity reagents to label these target molecules with a unique combination of dyes from the plurality of combination of dyes (S1), it would be easy to define a plurality of dyes (PD), which is large enough to ensure that the number of elements in S1>>20,000, i.e. several orders of magnitude higher such as 106 to 1010. In consequence, it is easily possible to define conditions in which the fraction □□ of actually assigned combinations of dyes to all available combinations of dyes from the plurality of combination of dyes (S1) becomes very small. In this case the likelihood to observe false-positive, i.e. combinations of dyes not assigned to a marker (type I false-positive) and/or combinations of dyes assigned to an affinity reagent not physically present in the readout volume (type II false-positive) subsumable under a first readout sequences becomes lower. If the conditions are such that a single iteration does not yield satisfactory levels of statistical confidence for presence calling for any of the following: combination of dyes; affinity reagents; and target molecules contained in the readout volume, it is possible to significantly improve the analysis in different ways which will be described herein.

In a particularly preferred embodiment of the invention, a first readout sequence is acquired in a first step and the “first set of combinations of dyes” subsumable under this first readout sequence is stored in a memory device. In a next step for at least some affinity reagents from the plurality of affinity reagents (A) the encoding/encryption might be changed. This can be done by deactivating the dyes introduced in the first step by means of eluting the affinity reagents, bleaching dyes or severing the linkage between the combination of dyes and the affinity reagents. Depending on the choice of method the target molecules are then re-labeled in a second step with at least some affinity reagents from the plurality of affinity reagents (A), wherein at least some affinity reagents are assigned to a different second combination of dyes. In a next step the “second set of combinations of dyes” is derived from a second readout sequence, i.e., a second readout sequence is generated in the same manner that the first readout sequence was generated and dyes from the second set of combinations of dyes identified in the second readout sequence. The retrieval of all second combinations of dyes subsumable under this second readout sequence is stored in a memory device. In a further step the “first set of combinations of dyes” and the “second set of combinations of dyes” might be compared to define the overlap and at least one statistical confidence measure is computed for each combination of dyes and/or affinity reagent and/or target molecule and/or analyte detected in the overlap. A certain combination of dyes and/or a certain affinity reagent and/or a certain target molecule and/or analyte is then said or called to be present in the readout volume, when the at least one statistical confidence measure computed for this particular certain combination of dyes and/or certain affinity reagent and/or certain target molecule and/or analyte is acceptable based on criteria, which may be fixed and a priori defined or dynamically derived and adjusted during the experiment.

In principle this process may be repeated until an acceptable level of statistical confidence in the acceptance or rejection of the presence of a certain combination of dyes and/or affinity reagents and/or target molecules and/or analytes of interest has been reached. Importantly, while in strict mathematical terms each iteration in this iterative process analyses exactly the same readout volume, it is possible and a particularly preferred embodiment of the present invention to allow small deviations (fractions 1/10000, 1/1000, 1/100, 1/10, ¼, ½ of the lateral extent of the effective PSF for example) in the spatial and/or temporal position (fractions 1/10000, 1/1000, 1/100, 1/10, ¼, ½ of the time a sample takes to traverse the lateral extent of the effective PSF for example) of the readout volume between a first and a second readout sequence. In this case a first readout sequence generates a priori knowledge about the second readout sequence in the sense of Bayesian probabilities according to the Bayesian theorem, this is not unlike pretest probability in diagnostic testing, in which a symptomatic patient typically has a substantially lower false-positive rate than an asymptomatic patient. In analog fashion one can argue that if an affinity reagent at was detected in a first readout volume then this influences its probability to be detected in an overlapping second readout volume, wherein the overlap may be understood as spatial or temporal.

Preferably, a “code” in the sense of this document may be for example a linear code (e.g. binary code), fixed length code, a variable length-code, or an error-correcting code. In a particularly preferred embodiment, the codes are “independent and identically-distributed”. In a particularly preferred embodiment of the present invention, a binary code is used.

“set of combinations of dyes subsumable under a readout sequence”: In the sense of this document “set combinations of dyes subsumable under a readout sequence” refers to the set containing all combinations of dyes from the plurality of combinations of dyes that can be subsumed under a certain readout sequence. The “set of combinations of dyes subsumable under a readout sequence” subsumable under a readout sequence contains □ elements.

“assignment rate”: This is the proportion of unique codes (also referred to as combination of dyes) from the set of unique codes, which may also be referred to as the plurality of combinations of dyes (S1), that are actually assigned to a marker and is denoted as □□.

Embodiments of the present invention provide a method and a device for analyzing a sample, preferably a biological sample that allows analyzing a very high number of markers in a very short time.

In accordance with embodiments of the invention, a method for analyzing a sample is provided, the sample comprising:
a plurality of affinity reagents, each affinity reagent being configured to attach to an analyte, at least one of the affinity reagents being attached to an analyte; and
a first plurality of combinations of dyes, each combination of dyes being unique within the first plurality of combinations of dyes and each combination of dyes comprising at least two dyes having different characteristics for at least one of: excitation and emission,
wherein at least one of the unique combinations of dyes is attached to an associated affinity reagent according to a first mapping,
wherein at least another one of the unique combinations of dyes is—preferably stochastically—attached to the associated affinity reagent according to a second mapping,
wherein the first mapping is different from the second mapping,
the method comprising for the first and for the second mapping in a time sequential manner:
   i) directing excitation light at the sample, the excitation light having characteristics for exciting at least the at least two dyes having different characteristics for at least one of: excitation and emission; and
   ii) generating at least one first readout from emission light emitted by the excited dyes.

In accordance with embodiments of the invention, a method for analyzing a sample is provided, the sample comprising:
a plurality of affinity reagents, each affinity reagent being configured to attach to an analyte, at least one of the affinity reagents being attached to an analyte; and
a first plurality of combinations of dyes, each combination of dyes being unique within the first plurality of combinations of dyes and each combination of dyes comprising at least two dyes having different characteristics for at least one of: excitation and emission,
a second plurality of combinations of dyes, each combination of dyes being unique within the second plurality of combinations of dyes and each combination of dyes comprising at least two dyes having different characteristics for at least one of: excitation and emission,
wherein the second plurality of combinations of dyes is different from the first plurality of combinations of dyes,
wherein at least one of the unique combinations of dyes of the first plurality of combinations of dyes—in particular a first subset of combinations of dyes—is attached to an associated affinity reagent according to a first mapping,
wherein at least one of the unique combinations of dyes of the second plurality of combinations of dyes—in particular a second subset of combinations of dyes—is attached to the associated affinity reagent according to a—preferably stochastically—second mapping,
wherein the first mapping is different from the second mapping,
the method comprising for the first and for the second mapping in a time sequential manner:
   i) directing excitation light at the sample, the excitation light having characteristics for exciting at least the at least two dyes having different characteristics for at least one of: excitation and emission;
   ii) generating at least one first readout from emission light emitted by the excited dyes.

Preferably, each unique combination of dyes in the first plurality of combinations of dyes is attached to only one affinity reagent, such that no unique combination of dyes is associated with more than one affinity reagent in the first mapping.

Preferably, generating at least one first readout comprises:
separating the emission light emitted by the excited dyes into detection channels, wherein the detection channels correspond to emission characteristics of the dyes.

Each combination of dyes might be selected to comprise one dye per detection channel.

Preferably, the at least two dyes have different excitation characteristics, and wherein excitation light having each excitation characteristic is directed to the sample at different times or simultaneously.

The method might further comprise:
providing the plurality of affinity reagents; and
providing the first plurality of combinations of dyes.

The method might further comprise:
providing the sample; and
I) attaching the plurality of affinity reagents to the first plurality of combinations of dyes to form a plurality of markers; and
   introducing the plurality of markers to the sample to allow attachment to analytes in the sample; or
II) introducing the plurality of affinity reagents to the sample to allow attachment to analytes in the sample; and
   attaching the plurality of affinity reagents to the first plurality of combinations of dyes, to form a plurality of markers attached to the analytes.

Preferably, attaching the plurality of affinity reagents to the first plurality of combinations of dyes comprises:
providing a plurality of linkers, each linker comprising a plurality of binding sites, each configured to bind to a dye; and
for each affinity reagent, attaching one linker to the affinity reagent, and binding one of the combinations of dyes to the linker, each dye from the combination being bound to a binding site.

The method might further comprise at least one of:
deactivating at least one of the dyes in the first plurality of combinations of dyes;
removing the attachment between at least one affinity reagent and at least one of the combinations of dyes;
removing the attachment between at least one affinity reagent and at least one of the analytes; and
waiting longer than a fluorescence lifetime of at least one of the dyes in the first plurality of combinations of dyes; and
repeating steps i) to iii) of claim 1 for a second, different, plurality of combinations of dyes or for the first plurality of combinations of dyes according to a second, different, mapping.

The method might further comprise:
suggesting, by a computer processor, at least one dye and/or combination of dyes for the second plurality of combinations of dyes and/or rules for the second mapping, based on the at least one first readout.

The method might further comprise iteratively repeating the steps as mentioned above for at least one of: a number of pluralities of combinations of dyes; and a number of mappings, until all affinity reagents attached to analytes in the sample are determined.

Preferably, the stochastic labeling is achieved by at least one of DNA-PAINT, DNA-Exchange, Exchange-PAINT, and by using oligonucleotide barcodes attached to affinity reagents and complementary attachment oligonucleotide portions attached to combinations of dyes, which are configured to allow dynamic association of combinations of dyes and their assigned affinity reagent.

The stochastic labeling might be achieved by at least one of DNA-PAINT, DNA-Exchange, Exchange-PAINT, and by using oligonucleotide barcodes attached to affinity reagents and complementary attachment oligonucleotide portions attached to combinations of dyes, which are configured to allow repeated association and dissociation of combinations of dyes and their assigned affinity reagent by at least one of the following:
a change of concentration of a host molecule;
a change of temperature;
a change of illumination condition;
a change in the concentration of at least one capture strand; and
a change in the concentration and/or activity of an enzyme.

Preferably determining, by at least one computer processor, at least one affinity reagent present in the sample, comprises:
comparing at least two readouts selected from: the at least one first readout, at least one second readout, and any further readouts generated in steps ii); and
determining the presence of at least one affinity reagent based, at least in part, on the comparison.

Preferably, determining the presence of at least one affinity reagent is based on at least one measure of statistical confidence.

Preferably, the characteristics of a dye for at least one of excitation and emission comprise at least one of: excited wavelength; emitted wavelength; fluorescence intensity; and fluorescence lifetime.

Preferably, determining, by at least one computer processor, at least one affinity reagent present in the sample based on a readout comprises:
converting the readout into a fully determined or overdetermined set of linear equations; and
solving the set of linear equations.

A device for analyzing a sample, might configured to perform the method as mentioned above.

A linker configured to bind to an affinity reagent may comprise:
at least one binding site configured to bind to at least one dye; and
an oligonucleotide attachment portion configured to bind to an oligonucleotide barcode of the affinity reagent.

The linker is configured to couple to an affinity reagent as mentioned above.

The linker may further comprise:
a plurality of binding sites, wherein at least two of the binding sites are configured to bind to dyes having different characteristics for at least one of: excitation and emission.

A reporter might comprise:
a linker as mentioned above; and
at least one dye bound to the at least one binding site of the linker.

Preferably, the linker may comprise a combination of dyes, each dye bound to one of the plurality of binding sites, wherein at least two of the dyes have different characteristics for at least one of: excitation and emission.

A marker might comprise:
an affinity reagent, configured to attach to an analyte; and
a reporter as mentioned above attached to the affinity reagent.

Preferably, the oligonucleotide attachment portion of the linker may be fully or partially complementary to the oligonucleotide barcode of the affinity reagent.

Preferably, the oligonucleotide attachment portion of the linker is configured to have an affinity to the oligonucleotide barcode of the affinity reagent such as to bind transiently under at least one condition before dissociating again from the oligonucleotide barcode. The at least one condition provides for dissociation of the oligonucleotide attachment portion depending on at least one of host-guest mediated ligand-invasion, strand displacement, and photochemical control of hybridization.

According to a particular embodiment, one of the oligonucleotide attachment portion of the linker and the oligonucleotide barcode of the affinity reagent comprises at least one guest molecule attached to a nucleobase thereof and configured to complexate with a host molecule, and the other of the oligonucleotide attachment portion of the linker and the oligonucleotide barcode of the affinity reagent comprises at least one nucleobase complementary to the nucleobase to which the guest molecule is attached.

Preferably, the host molecule may be a cucurbituril such as CB[7].

According to another embodiment, the marker may comprise at least one base mismatch between the oligonucleotide attachment portion of the linker and the oligonucleotide barcode of the affinity reagent hindering stable association therebetween, wherein the base mismatch is configured to be bridged by photoswitching.

For example, the base mismatch is a GG mismatch which is configured to be bridged by photoswitching trans-NCDA to cis-NCDA.

According to a preferred embodiment, the affinity reagent is an oligonucleotide-probe which is configured to bind to a nucleic acid analyte.

A plurality of markers as mentioned above may be provided, wherein each reporter comprises a unique combination of dyes, and wherein each reporter is attached to an affinity reagent configured for attachment to an analyte, such that no unique combination of dyes is associated with more than one affinity reagent.

A solution may comprise a linker as mentioned above, a reporter as mentioned above, a marker as mentioned above, or a plurality of markers as mentioned above.

A lyophilized solid may comprise a linker as mentioned above, a reporter as mentioned above, or a marker as mentioned above, or a plurality of markers as mentioned above.

A computer program with a program code is provided for performing the method as mentioned above when the computer program is run on a processor.

A computer readable storage medium storing the computer program as mentioned above is provided.

A database is provided, the database comprising information corresponding to:
a plurality of affinity reagents;
a first plurality of combinations of dyes; and
a first mapping,
and optionally further comprising information corresponding to at least one of: a second and/or any further plurality of combinations of dyes; each combination of dyes having characteristics for at least one of: excitation and emission of the dyes; a second mapping or any further mapping; at least one first readout; at least one second readout and/or any further readout;
wherein the database is used to carry out at least one of steps i) to ii) of the method as mentioned above.

Furthermore, there may be provided a method for analyzing a sample, the sample comprising: a plurality of affinity reagents, each affinity reagent being configured to attach to an analyte, at least one of the affinity reagents being attached to an analyte; and a first plurality of combinations of dyes, each combination of dyes being unique within the first plurality of combinations of dyes and each combination of dyes comprising at least two dyes having different characteristics for at least one of: excitation and emission, wherein each one of the unique combinations of dyes is attached to an associated affinity reagent according to a first mapping, the method comprising:
i) directing excitation light at the sample, the excitation light having characteristics for exciting at least the at least two dyes having different characteristics for at least one of: excitation and emission;
ii) generating at least one first readout from emission light emitted by the excited dyes; and
iii) determining, by at least one computer processor, at least one affinity reagent present in the sample based on the at least one first readout.

The method may be a computer-implemented method.

In this way, the method provides an improved method for detecting the presence of analytes in a sample. In particular, by directing excitation light having characteristics for exciting dyes having different excitation and/or emission characteristics, the readout generated can contain information allowing for the determination of a greater number of analytes per readout than is possible using known methods, as will be described in greater detail herein.

The method may be further defined in that each unique combination of dyes in the first plurality of combinations of dyes is attached to only one affinity reagent, such that no unique combination of dyes is associated with more than one affinity reagent in the first mapping. In this way, the detection of a unique combination of dyes within a readout can, with confidence, be used to determine that an analyte is present in the sample. It may also be said that the mapping of the plurality of combinations of dyes to affinity reagents is at least injective, preferably bijective.

Generating at least one first readout may further comprise: separating the emission light emitted by the excited dyes into detection channels, wherein each detection channel substantially corresponds to a dye from the plurality of dyes or wherein the detection channel corresponds to a dye from the plurality of dyes. Each combination of dyes may be selected to comprise one dye per detection channel.

In this way, a greater number of dyes may be readout on a readout device, as will be described in greater detail herein.

The at least two dyes may have different excitation characteristics, and wherein excitation light having each excitation characteristic is directed to the sample at different times. In this way, the information available to the readout device can be increased, and many more combinations of dyes can be unique. For example, dyes having different excitation characteristics and the same emission characteristics may be harder to distinguish from one another if light having both of their excitation characteristics is directed towards the sample simultaneously. By separating the excitation lights, and noting when light having the shared emission characteristic was emitted, the dyes can be more easily distinguished. This leads to a greater number of feasible combinations of dyes.

The at least two dyes may have different excitation characteristics, and wherein excitation light having each excitation characteristic is directed to the sample simultaneously. In this way, the method may be more efficient both computationally and in terms of time to run. Exciting all dyes at the same time may be permissible for smaller numbers of analytes in the sample, i.e., where fewer unique combinations of dyes are required.

The method may further comprise: providing the plurality of affinity reagents; and providing the first plurality of combinations of dyes.

The method may further comprise:
providing the sample; and
   I) attaching the plurality of affinity reagents to the first plurality of combinations of dyes to form a plurality of markers; and
introducing the plurality of markers to the sample to allow attachment to analytes in the sample; or
   II) introducing the plurality of affinity reagents to the sample to allow attachment to analytes in the sample; and
attaching the plurality of affinity reagents to the first plurality of combinations of dyes, to form a plurality of markers attached to the analytes.

In this way, it can be controlled how the attachments between affinity reagent and analyte, and between combination of dyes or reporter and affinity reagent, are achieved. Depending on the affinity reagent, analyte, and dyes, among other variables, it may be advantageous to form the markers before attachment to the analytes. In other cases, it may be advantageous to attach the affinity reagents to the analytes, and add the dyes later, forming the markers “in situ” within the sample.

Attaching the plurality of affinity reagents to the first plurality of combinations of dyes may comprise:
providing a plurality of linkers, each linker comprising a plurality of binding sites, each configured to bind to a dye; and
for each affinity reagent, attaching one linker to the affinity reagent, and binding one of the combinations of dyes to the linker, each dye from the combination being bound to a binding site.

The method may further comprise at least one of:
deactivating at least one of the dyes in the first plurality of combinations of dyes;
removing the attachment between at least one affinity reagent and at least one of the combinations of dyes;
removing the attachment between at least one affinity reagent and at least one of the analytes; and
waiting longer than a fluorescence lifetime of at least one of the dyes in the first plurality of combinations of dyes; and
repeating steps i) to iii) as above for a second, different, plurality of combinations of dyes or for the first plurality of combinations of dyes according to a second, different, mapping.

In this way, more information can be generated that pertains to the same sample. A single combination of dyes and/or single mapping may not produce a readout from which any or all analytes can be determined. It is therefore desirable to obtain more information relating to the original sample. The method may therefore deactivate, or allow to deactivate, at least one of the dyes or attachments, such that the (second) readout generated when steps i) to iii) are run again will be different to the original (first) readout. A second readout having new information relative to the first can then be used to determine further analytes.

The method may further comprise suggesting, by a computer processor, at least one dye and/or combination of dyes for the second plurality of combinations of dyes and/or rules for the second mapping based on the at least one first readout. In this way, the number of iterations can advantageously be reduced, thereby optimizing the process.

The method may further comprise iteratively repeating the steps of at least one of deactivating, removing attachment, waiting, and suggesting for at least one of: a number of pluralities of combinations of dyes; and a number of mappings, until all affinity reagents attached to analytes in the sample are determined. In this way, the method determines all analytes present in the sample.

Determining, by at least one computer processor, at least one affinity reagent present in the sample, may comprise:
comparing at least two readouts selected from: the at least one first readout, at least one second readout, and any further readout generated in steps ii); and
determining the presence of at least one affinity reagent based, at least in part, on the comparison.

In this way, the method can analyse differences between the readouts, and the known differences between the mappings and/or combinations of dyes, to aid the determination of analytes in the sample.

Determining the presence of at least one affinity reagent may be based on at least one measure of statistical confidence. Measures of statistical confidence may be employed at any number of, including only one, stages of the method. For example, a measure of statistical confidence may be used to determine whether or not a dye has been excited, the same or a different measure may then be used to determine the presence or not of a particular dye or of a combination of dyes, and the same or a different measure may be used to determine the presence of an analyte based on the determined presence of a combination of dyes. This process of “presence calling”, and certain exemplary probability thresholds, are described in greater detail herein.

The characteristics of a dye for at least one of excitation and emission may comprise at least one of: excited wavelength; emitted wavelength; fluorescence intensity; and fluorescence lifetime. In this way, the readout can effectively be retrieved and analysed, and the dyes distinguished from one another.

Determining, by at least one computer processor, at least one affinity reagent present in the sample based on a readout may comprise:
converting the readout into a fully determined or overdetermined set of linear equations; and
solving the set of linear equations.

In this way, the method provides a computationally efficient way to “decode” the information in each readout, such that the presence of analytes in the sample can be determined.

In accordance with embodiments of the invention, there is provided a device for analyzing a sample, the device being configured to perform the method according to embodiments of the invention.

In accordance with embodiments of the invention, there is provided a linker, configured to couple to an affinity reagent, the linker comprising:
a plurality of binding sites, wherein at least two of the binding sites are configured to bind to dyes having different characteristics for at least one of: excitation and emission.

Preferably, the linker is configured to perform the method according to embodiments of the invention or the method as described herein.

In accordance with embodiments of the invention, there is provided a reporter, comprising:
a linker according to embodiments of the invention; and
a combination of dyes, each dye bound to one of the plurality of binding sites, wherein at least two of the dyes have different characteristics for at least one of: excitation and emission.

In accordance with embodiments of the invention, there is provided a marker, comprising:
an affinity reagent, configured to attach to an analyte; and
a reporter according to embodiments of the invention, attached to the affinity reagent.

In accordance with embodiments of the invention, there is provided a plurality of markers according to embodiments of the invention, wherein each reporter comprises a unique combination of dyes, and wherein each reporter is attached to an affinity reagent configured for attachment to an analyte, such that no unique combination of dyes is associated with more than one affinity reagent.

In these ways, embodiments of the invention provide the building blocks to put methods in accordance with embodiments of the invention into effect. The linker, reporter, marker, and plurality of markers described above allow the advantageous effects, including determination of a greater number of analytes per readout, as described in relation to the method.

In accordance with embodiments of the invention, there is provided a solution comprising at least one of a combination of dyes according to embodiments of the invention, a linker according to embodiments of the invention a reporter according to embodiments of the invention, a marker according to embodiments of the invention, and a plurality of markers according to embodiments of the invention. A solution may be manufactured to comprise linkers, reporters, markers, or a plurality of markers, by any suitable methods apparent to a skilled person. By way of example only, the solution may comprise water and/or saline, for example a phosphate-buffered saline, and may comprise further minerals in alternative formulations.

In accordance with embodiments of the invention, there is provided a lyophilized solid comprising at least one of a combination of dyes according to embodiments of the invention, a linker according to embodiments of the invention, a reporter according to the embodiments of invention, a marker according to embodiments of the invention, and a plurality of markers according to embodiments of the invention. A lyophilized solid may be manufactured to comprise linkers, reporters, markers, or a plurality of markers, by any suitable methods apparent to a skilled person. By way of example only, a lyophilized solid may be manufactured by processes of freezing and drying, optionally under vacuum.

In accordance with embodiments of the invention, there is provided a computer program with a program code for performing the method according to embodiments of the invention when the computer program is run on a processor.

In accordance with embodiments of the invention, there is provided a computer readable storage medium storing the computer program according to embodiments of the invention.

In accordance embodiments of with the invention, there is provided a database comprising information corresponding to:
a plurality of affinity reagents;
a first plurality of combinations of dyes; and
a first mapping,
and optionally further comprising information corresponding to at least one of: a second and/or any further plurality of combinations of dyes; each combination of dyes having characteristics for at least one of: excitation and emission of the dyes; a second mapping or any further mapping; at least one first readout; at least one second readout and/or any further readout;
wherein the database is used to carry out at least one of steps i) to iii) as described above.

In this way, embodiments of the invention may keep track of the relevant information in an efficient way, which allows the rapid retrieval and storage requiring minimal amounts of memory on a memory device.

In further exemplary embodiments, a method for analyzing a biological sample comprises the following steps:
a) providing a plurality of affinity reagents (S2), wherein each affinity reagent (a1, a2, a3, . . . an) of the plurality of affinity reagents (S2) is configured to specifically bind to a predetermined target structure within the biological sample or to a predetermined chemical compound or to a predetermined chemical element;
b) providing a plurality of combinations of dyes (S1) [from a plurality of dyes (YD) with y□ members], each combination of dyes (s1, s2, s3, . . . sn) is unique within the plurality of combinations of dyes (S1), each combination of dyes (s1, s2, s3, . . . sn) comprises at least two different dyes (|s|>=2);
c) wherein the plurality of combinations of dyes (S1) is composed such that each dye (y1, y2, y3, . . . y□) in the plurality of combinations of dyes (S1) can be readout by a readout device; wherein dyes can be separated by a readout device into channels; each channel corresponding to one of the dyes (y1, y2, y3, . . . y□); This means that each dye can be discerned from the other dyes in the same by the readout that is used for data acquisition based on any of the following properties or their combination: excitation fingerprint/spectrum, emission spectrum, intensity, fluorescence lifetime, fluorescence anisotropy, and an affinity reagent unique to the marker, the affinity reagent being configured to attach to a predetermined structure within the sample. In a particularly preferred embodiment of the invention the method disclosed in PCT/EP2021/063310, to which we refer to as “IP” (iterative hyperplexing) is used to substantially increase the y□□ the overall number of dyes that can be reliably discerned from each other and represented in distinct channels by a suitably configured readout device. This is particularly advantageous as even small increases in the y□□ such as from y□STD□□ (standard widefield fluorescence microscope) without the method to y□IHP□□□□ using “IHP” lead to substantially higher cardinalities of the pluralities of combinations of dyes (S1) that can be generated using different functions or algorithms to compose the combinations of dyes. These substantial increases in the cardinalities of the pluralities of combinations of dyes (S1) lead to exponentially higher statistical power of the approach and are the foundation for decoding a very high number of combinations of dyes present in a readout volume with high statistical confidence in the decoding result, i.e. the presence calling.
d) preferably introducing at least some affinity reagents from the plurality of affinity reagents (A) to the sample; The plurality of affinity reagents may be introduced in entirety or in several iterative steps. In the latter case sub-pluralities of affinity reagents may be defined using a priori knowledge to optimize the conditions for presence calling.
e) before or after Step d), preferably assigning each affinity reagent of the plurality of affinity reagents (S2) to at least one combination of dyes from the plurality of combinations of dyes (S1); both the virtual assigning (virtual marker) and the physical constitution of a marker (physical marker), i.e. the physical assembly of the linkage between the affinity reagent and its assigned combinations of dyes may be performed before the introduction of the affinity reagent into the sample. In a particularly preferred embodiment of the invention, affinity reagents are introduced into the sample and allowed to attach to their predetermined target structures before the linkage between the affinity reagent and the assigned combination of dyes is established. In another particularly preferred embodiment of the invention, a linkage between an affinity reagent and its first assigned combination of dyes from a first round in an iterative process, in which steps d)-f) are repeated for at least two iterations, is severed to allow establishing of a new linkage between said affinity reagent and a second assigned combination of dyes in a second round. This may be referred to as “code swapping” or introducing a further mapping or changing the encoding of affinity reagents and/or markers, which is a powerful strategy to decode a readout volumes even if, the sample contains a very high number of different combination of dyes and associated affinity reagents.
f) preferably directing excitation light having the respective specific characteristics for exciting each dye in the plurality of combinations of dyes to the sample in order to excite the respective dyes
g) preferably generating at least one readout from emission light emitted by the excited dyes located in a readout volume of the sample by the detection channels for at least one readout volume; and
h) preferably determining which affinity reagents are present in the readout volume based on the at least one readout

In a particularly preferred embodiment of the invention the determination of the presence of affinity reagents in the readout volume is established based on a measure of statistical confidence and a certain level of statistical confidence.

A measure of statistical confidence is computed for each marker and/or affinity reagent and/or combination of dyes and/or predetermined target molecule. This may be a combined measure consisting of multiple measures of statistical confidence assessing related aspects. The measure of statistical confidence may incorporate a priori knowledge and use Bayes theorem for example to adjust the probability of observing a given marker and/or affinity reagent and/or combination of dyes and/or predetermined target molecule based on a priori knowledge about that marker and/or affinity reagent and/or combination of dyes and/or predetermined target molecule. This a priori knowledge may be generated before or during the experiment. This means that for example a p value is computed for each marker and/or affinity reagent and/or combination of dyes and/or predetermined target molecule which assesses the probability that the detected presence (qualitative decoding) and/or quantity (relative or absolute quantitative decoding) is observed when the null hypothesis is true, i.e. the respective marker and/or affinity reagent and/or combination of dyes and/or predetermined target molecule is actually not present in the readout volume. The presence calling, i.e. the user's decision to accept the presence of a given marker and/or affinity reagent and/or combination of dyes and/or predetermined target molecule in the readout volume is then based on attaining a sufficient level of statistical confidence. The decision may be automated by using thresholds, which may be fixed and the same across all markers, affinity reagents, combinations of dyes, and target molecules or they may be different thresholds, which may be based on a priori knowledge. Further thresholds may be adjusted dynamically throughout the experiment. For example, they may be made more or less stringent. This is advantageous as it allows to demand a higher statistical confidence for target molecules, which are of particular interest.

Embodiments of the present invention are based on “looking at” microscopy as an encoding/decoding problem rather than a problem of registering spatially located intensities in an image, which is essentially a matrix of intensity values. While the method described according to embodiments of the present invention is compatible with image-based readouts the “images” generated by the method and device according to embodiments of the present invention should be regarded probabilistic mathematical models of the reality of the sample under investigation, in which the presence of a target molecule is detected or called (presence calling) based upon the decision of the user to accept its presence based on a measure of statistical confidence and a certain level of statistical confidence in the presence of the respective target molecule in the readout volume.

Making the step of accepting the presence of a certain marker, a certain affinity reagent, and a certain target molecule based on a measure of statistical confidence and a certain level of statistical confidence (i.e. presence calling) generates a mathematical truth. This is an important aspect of the present invention as this also implies that following to presence calling one is operating in the axiomatic domain of mathematics which is inherently free of influences that complicate measurements in the non-mathematical domain, i.e. the physical, chemical, biological domain. This fact therefore has important implications and also indicates that this method enables a completely new microscopic modality.

The statistical methods to provide a measure of statistical confidence on a per marker-basis or per target-molecule basis, may well be a combined measure and may in many ways be similar or identical to methods, which are used in transcriptomics and genomics, where enrichment scores and p values are commonly used.

Embodiments of the present invention relate to the patent application with the title “Method and device for analyzing a biological sample” with the application number PCT/EP2021/063310 which leverages the capability of the “IHP method” to image or readout a plurality of dyes with a high number of dyes in a single round. In contrast to the method disclosed in the patent application with the title “Method and device for analyzing a biological sample” with the application number PCT/EP2021/063310, wherein there is a 1:1 relationship between a given dye and a given affinity reagent, such that each marker is unique in a round, here we disclose a method in which this principle is combined with a combinatorial code such that there is 1:many relationship between marker and dyes. The content of PCT/EP2021/063310 is completely included herein by reference.

In a particularly preferred embodiment of the method, a given affinity reagent, such as an antibody, a single domain antibody, an oligonucleotide probe, an aptamer or a toxin, is assignable to a reporter comprising a unique combination of dyes and a linker forming a virtual marker, i.e. wherein the bijective pair or injective association between an affinity reagent (ai) and a combination of dyes corresponds to a marker (μi) within a plurality of markers (M).

Further in this embodiment dyes in the plurality of dyes can be assigned to sets of dyes A to n (n being 0 or an element of the natural numbers), wherein each set of dyes A to n contains yA to yn dyes, such that the plurality of dyes (PD) contains yA+yB+yC+ . . . . yn=y□□ members; wherein dyes assigned to one of the sets of dyes A to n are excitable with the same excitation light, e.g. with excitation light having respective different wavelengths λ1 to λn, wherein at least all dyes in each set of dyes A to n can be separated by a readout device into channels, wherein each dye is read out in an individual channel and each channel corresponds to one of the dyes; Using the dyes and the readout device in this way, which is based on the “IHP method” maximizes the number of dyes that can be readout and separated reliably, i.e. increases y□□ and thereby the cardinality of the plurality of combinations of dyes (S1). The unique combination of dyes (si) is in this case assigned to the affinity reagent (ai) either prior to or following to the introduction of the affinity reagent into the sample, but prior to the generation of the readout. In a next step at least some affinity reagents from the plurality of affinity reagents (S2) are introduced into the sample. Then excitation lights are directed to the sample in order to excite the fluorescent dyes of the markers (μ1, μ2, μ3, . . . μn), this means that all dyes in the plurality of combinations of dyes (S1) are excited at least one time. Following excitation of the dyes at least one readout, preferably a complete readout, from fluorescence light emitted by the excited dyes located in a readout volume of the sample is generated, the readout comprising at least two channels, each channel corresponding to one of the dyes. In other words in the method described in this document each dye is readout in an individual channel. In a following step the markers present in the readout volume are determined based on the at least one readout sequence obtained in step (d) which may be made dependent on a measure of statistical confidence and attaining a certain level of statistical confidence (“presence calling”).

In a particularly preferred embodiment of the method, the plurality of combinations of dyes (S1) is mapped uniquely to the plurality of affinity reagents (A=S2=T1*) using at least one code C□1 to C□n and/or at least one cipher X□1 to X□n, wherein C: S->T* or X: S->T* are total functions which are preferably bijective or at least injective, wherein S is the “source alphabet” and T is the “target alphabet”, and wherein S and T are finite sets [case Q].

In a particularly preferred embodiment of the method, the plurality of affinity reagents (A=S2=T1*) is mapped uniquely to the plurality of combinations of dyes S1 using at least one code C□1 to C□n and/or at least one cipher X□1 to X□n, wherein C: S->T* or X: S->T* are total functions which are preferably bijective or at least injective, wherein S2 is the “source alphabet” and T1 is the “target alphabet”, and wherein S2 and T1 are finite sets [case □].

In both cases □ and □ the method disclosed in this document can be performed so as long as the codes C1, C2, C3, . . . Cn, and/or ciphers X1, X2, X3, . . . Xn used for encoding/decoding and/or encrypting/decrypting are total functions that are either injective or bijective. In the following preferred embodiments are proposed as an example, which use a certain code Ci, but this is not intended to a express a restriction of the method to any particular code in anyway, as all the codes C1, C2, C3, . . . Cn, and/or ciphers X1, X2, X3, . . . Xn., which are total functions and are either injective or bijective may be used for encoding/decoding and/or encrypting/decrypting to perform the method disclosed in this document.

There may be a 1: n relationship between a given affinity reagent and the dyes assigned to or attached to it, as combination of dyes contains at least two dyes. Importantly, n in this case defines the number of different dye species that are being used not the number of dye molecules.

In a particular preferred embodiment, of the present invention the combination of dyes is established by randomly selecting one dye from A to n sets of dyes, with yA+yB+yC . . . +yn=y□□□ members. If for example the method disclosed in “IHP method” is used and each set A to n comprises yA to yn markers, and n different excitation lights are used for generating n different readouts, the number of unique markers that can be readout and discerned from each other is yA+yB+yC . . . +yn. If for example the method disclosed in this document is used and each marker is labeled with n dyes, and n different excitation lights are used for generating n different images, the number of unique markers that can be readout and discerned from each other is yA×yB×yC . . . ×yn. In other words this leads to yA×yB×yC . . . ×yn unique codes. We refer to this particularly preferred embodiment as “set-based encoding”.

In a particularly preferred embodiment of the present invention the plurality of dyes (YD) formed by all fluorescent dyes of the plurality of combination of dyes (S1) comprises at least 10, 20, 50, 100, 1000, or 10000 different fluorescent dyes.

In a further particularly preferred embodiment of the method, a given affinity reagent, such as an antibody, a single domain antibody, an oligonucleotide probe, an aptamer or a toxin, is connected to a set of up to n×y labels such that there is variable and random relationship between a given affinity reagent and the dyes attached to it. This leads to a binary encoding, in which the absence of a particular dyes is counted as “0” and the presence as “1” in the code with up to n×y digits generating a set of unique codes with 2(yA+yB+yC . . . +yn) or 2y□□□ members. We refer to this particularly preferred embodiment as “binary encoding”.

Thus, the method described in this document vastly increases the number of markers and/or combinations of dyes and/or affinity reagents and/or predetermined target structures that can be readout without requiring to remove or to deactivate the previous markers, and without additional staining.

Importantly, there are many other codes or ciphers, i.e. ways of encoding/encrypting and decoding/decrypting that can be used in conjunction with the method disclosed in this document.

Each affinity reagent targets its combination of dyes to its predetermined structure which may also be named a target molecule or an analyte within the biological sample or lysate, e.g. a specific biomolecule.

Owing to combinatorial encoding for both methods the number of combinations of dyes which can be regarded as unique codes grows exponentially and quickly exceeds the number of protein-coding genes, which is in the range of 20,000. This is an important reference value as proteins carry out the majority of biological functions and are therefore of great interest. For this reason, the bulk of fluorescence microscopy performed today analyzes protein targets.

In both embodiments, the method remains compatible with various means of amplification including multiple binding sites or amplification strategies based on enzymatic reactions such as for example rolling circle DNA amplification.

In both embodiments, the method remains compatible with various analyte classes as the affinity reagent can be for example an antibody (protein target) or an oligonucleotide (RNA/DNA target).

In a particularly preferred embodiment of the present invention each marker (μi) comprises a linker having at least two different attachment sites, the combination of attachment sites being unique to the marker; and wherein each dye is connected to a complementary linker to form a reporter, the complementary linker being unique to the dye and configured to attach to a predetermined attachment site.

In a particularly preferred embodiment of the present invention the linker and/or the complementary linkers are oligonucleotides comprising DNA, RNA, peptide nucleic acid, morpholino or locked nucleic acid, glycol nucleic acid, threose nucleic acid, hexitol nucleic acid, or another form of artificial nucleic acid.

In a particularly preferred embodiment of the present invention, the linker and/or complementary linkers contain a site for enzymatic cleavage or photolysis. This allows the efficient and particularly easy releasing of a first combination of dyes in order to deactivate the first combination of dyes, which may be followed by relabeling with a second combination of dyes.

In a preferred embodiment of the present invention, the reporters are attached to their respective attachment sites before the markers are introduced into the sample.

In a particularly preferred embodiment of the present invention, at least two readouts are generated; and the reporters are dynamically associated with and/or dissociated from their respective attachment sites between the generation of the first and second readouts in order to achieve a stochastic labeling. This is a strategy to increase spatial resolution and a strategy to render the decoding of multi-species readout volumes simpler. Such stochastic labeling may be based on super resolution microscopy such as STORM, PALM, GSDIM or a related method which leverages blinking.

In a particularly preferred embodiment of this invention, stochastic labeling is achieved by combining the method with DNA-PAINT.

In a particularly preferred embodiment of this invention, the plurality of dyes formed by all fluorescent dyes of the markers is divided into sets of dyes A to n, with yA to yn members, with yA+yB+yC+ . . . yn=y□□□ with y being a natural number and y□□ being the total number of dyes in the plurality of dyes (PD); wherein each dye in the same set can be excited by light of essentially one wavelength spectrum or by the same wavelength spectrum; wherein at least one excitation light for each set of dyes is directed at the sample in order to excite the fluorescent dyes of the respective set; wherein at least one readout for each set of dyes is generated from fluorescence light emitted by the excited dyes located in the readout volume of the sample, the readout comprising at least two channels, each channel corresponding to one of the dyes of the respective set. This embodiment uses the “IHP method”, which basically allows a higher number of dyes to be readout on a readout device. This is particularly advantageous as a higher y□□ leads to a higher cardinality of the plurality of combinations of dyes (S1) and consequently to a lower assignment rate □ and higher statistical power of the method.

In a particularly preferred embodiment of the present invention, excitation lights for exciting the sets of dyes A to n are directed onto the sample in a sequence temporally following each other.

In a particularly preferred embodiment of the present invention, the readout is an image, or a microscopic image, or a readout image data stream of the readout volume.

In a particularly preferred embodiment of the present invention, the readout is or contains a hyperspectral image of the sample. This is particularly advantageous as it allows a high total number of dyes y□□ to be used and leads to a higher cardinality of the plurality of combinations of dyes (S1) and consequently lower assignment rate □ and higher statistical power of the method.

In a particularly preferred embodiment of the present invention, the method comprises the further step of stabilizing the fluorescence lifetime of at least one fluorescent dye. This can be achieved by placing the fluorescent dye in a shielded environment by at least one of encapsulating, polymer-matrix embedding, and co-crystallizing. SMILEs are a particularly advantageous class of dyes in this regard. In a particularly preferred embodiment of the present invention, at least one dye in the plurality of dyes (PD) is a SMILEs.

In a particularly preferred embodiment of the present invention, the step of generating the channels is based on at least one of channel unmixing, spectral unmixing, excitation spectral imaging, spectral phasor analysis, spectral FLIM phasor, a fluorescence lifetime of the fluorescent dyes and an excitation fingerprint of the fluorescent dyes. This is particularly advantageous as it allows a high total number of dyes y□□ to be used and leads to a higher cardinality of the plurality of combinations of dyes (S1) and consequently lower assignment rate □ and higher statistical power of the method.

In a particularly preferred embodiment of the present invention, the step of generating the channels is based on at least two orthogonal contrasts. When orthogonal contrasts are obtained from these methods and used in conjunction, they can be used to strongly increase the total number of dyes y□□ i.e. separate a much higher number of dyes. For example, excitation fingerprinting information may be combined with fluorescence emission spectral information and/or fluorescence lifetime information with either or both of the aforementioned.

In a particularly preferred embodiment of the present invention, the step of generating the channels is based on at least one of machine learning, deep learning, or artificial intelligence.

In a particularly preferred embodiment of the present invention, the following steps are repeated at least twice in order to create series of images or readouts of the sample: providing a second plurality of markers, introducing the second plurality of markers into the sample, direct the at least one excitation light onto the sample, generating the at least one readout, and determining the markers present in the readout volume; or wherein the steps a) to e) of the methods described above are repeated at least twice.

In a particularly preferred embodiment of the present invention, the reporters labeling the second plurality of markers comprise combinations of dyes that were determined based on the first series of images or readouts of the sample.

In a particularly preferred embodiment of the present invention, the reporters are assembled by adding a mix of dyes wherein each dye is connected to a complementary linker to form reporters with linker molecules containing dye-specific attachment sites for all dyes in the plurality of dyes, such that adding a mix of dyes corresponding to a unique combination of dyes to a linker molecule in a coupling reaction volume leads to a stoichiometric coupling.

In a particularly preferred embodiment of the present invention, the reporters are assembled by adding a mix of dyes wherein each dye is connected to a complementary linker to form reporters with linker molecules containing dye-in-specific attachment sites for all dyes in the plurality of dyes, such that adding a mix of dyes corresponding to a unique combination of dyes to a linker molecule in a coupling reaction volume leads to a stochastic coupling.

In a particularly preferred embodiment of the present invention, the excitation light is coherent light.

In a particularly preferred embodiment of the present invention, the excitation light comprises a wavelength range being smaller than 50 nm, smaller than 30 nm, smaller than 10 nm or a single wavelength.

In a particularly preferred embodiment of the present invention, a device for analyzing a biological sample is adapted to carry out the method according to one of the methods described above.

In a particularly preferred embodiment of the present invention the device comprises a microscope preferably a lens-free microscope, a light field microscope, widefield microscope, a fluorescence widefield microscope, a light sheet microscope, a scanning microscope, or a confocal scanning microscope, a plate reader, a cytometer, an imaging cytometer, or a fluorescence activated cell sorter configured to generate the at least one readout.

In a particularly preferred embodiment of the present invention the device is configured to determine a fluorescence emission intensity, a fluorescence lifetime, an emission spectrum, an excitation fingerprint, fluorescence anisotropy from fluorescence dyes in the sample.

In a particularly preferred embodiment of the present invention the device is configured to perform separation of the readout into the at least two channels by at least one of a spectrometer comprising a prism or a grating and at least one detector.

In a particularly preferred embodiment of the present invention the device is configured to perform separation of the readout into the at least two channels by at least one of a spectrometer comprising a prism or a grating and at least one detector and the device comprises a comprising a time-sensitive detector.

In a particularly preferred embodiment of the present invention, the device may comprise a memory device for storing a unique identifier that identifies the affinity reagent, the predetermined structure, and the unique combination of dyes for each marker.

In a particularly preferred embodiment of the present invention, the device may comprise a calibration unit configured to receive the fluorescence light emitted by the excited dye, and to generate calibration data based on the received fluorescence light; wherein the at least one readout is generated based on the calibration data.

In a particularly preferred embodiment of the present invention, no combination of dyes is assigned to more than one affinity reagent.

The following examples illustrate the power of the approach and likewise underscore its feasibility. In the following example n=5 different excitation light wavelengths, e.g. 405 nm, 488 nm, 560 nm, 630 nm, 700 nm are used to excite n=5 sets of fluorescent dyes with yA=yB=yC=yD=yE=5 members, such that the plurality of different dyes y□□ used in this example is 25. In this case, owing to the exponential nature of combinatorial coding the total number of unique codes is 55=3125. In other words, the method presented here can be used to readout the entire human secretome based on a simple 5 channel fluorescence-based readout such as a microscope or a cytometer using commercially available dyes. In relation to the prior art, which enables multiplexing in the range of 30-60 biomarkers using an iterative process (i.e. ~markers per round), this is a significant 50-100× improvement. Using the same set of dyes and an iterative process in which the sample is stained, imaged, and then blanked (i.e. dyes are being removed or inactivated) it would be possible to probe the 30,000 targets in 10 rounds, which is roughly equivalent to the number of coding genes in the human genome. Today about 20,380 human protein-coding genes have been registered in UNIPROT.

In the following example n=6 different excitation lights, e.g. 350 nm, 405 nm, 488 nm, 560 nm, 630 nm, 700 nm are used to excite n=10 sets of fluorescent dyes with yA=yB=yC=yD=yE=yF=yG=yH=yI=yJ=10 members, such that the plurality of different dyes y□□ used in this example is 60. In this case, owing to the exponential nature of combinatorial coding the total number of unique codes is 106=1,000,000. In other words, the method presented here can be used to readout the entire human proteome estimated to have on the order of 80,000-400,000 distinct proteins in a single round.

Further, the method is easily adapted in a cytometer, a plate reader, a fluorescence microscope, allowing to readout a very high number of markers. In other words, the method is compatible with image-based and non-image-based readouts.

Further, the method is easily adapted in a fluorescence microscope, allowing to readout a very high number of markers with very high spatial resolution.

In a preferred embodiment the method is based on detecting individual disparate spots, i.e. spots that can be resolved from each other by the readout device. A very high number of spots can be readout at the same time using area detectors that image a field of view, such as for example a camera. Disparity of spots may result from different locations in X, Y direction in a single field of view in an image of a microscope, or from a different point of time T in passing through a flow cell as for example in a cytometer or imaging cytometer or in a laser scanning microscope. In a system which uses image-based readouts such as microscopes and plate readers disparity in densely labeled structures such as, when trying to image a very high number of markers in a cell for instance, may be achieved by stochastic dye blinking, i.e. the temporal separation. This is a commonly used strategy in stochastical optical reconstruction microscopy (STORM) and related modalities, which rely on for example Gaussian fitting to find the location of disparate emitters in densely labeled samples.

The disparity of spots maybe an immediate consequence of the assay format such as in a bead-based assay in flow-through, wherein a plurality of beads passes the flow cell (to which a readout device is adapted) in sequence. A spot may result from a structure which is bigger or smaller than the readout volume. In a biological sample like a cell, intracellular targets may be at different X, Y, Z locations. In some cases, when label density is too high, an iterative approach may be employed to reduce the label density to an acceptable level. Other strategies to achieve disparity of spots may involve stochastically labeling by reducing the concentration of labeling reagent for instance. Further disparity may be achieved by expanding the sample using protocols known as expansion microscopy, which are described in Wassie et al. 2019 Nature Methods, Volume 16, pages 33-41 (2019). Further a suitable spacing between spots may be achieved by increasing the number of sets of dyes n. To this end tunable light sources or continuous light sources can be combined with the method. Further strategies for densely labeled samples are discussed in the following.

Combining the Disclosed Method with Iterative Staining Processes

In another preferred embodiment the following steps are repeated at least twice in order to create a series of readouts/images of the sample: Staining the sample. Directing the first excitation onto the sample. Generating the first readouts/images. Directing the second excitation onto the sample. Generating the second readouts/images. The steps defined in claim 1 describe a single round of readouts/images acquisition. Additional rounds may be performed in order to acquire a series of readouts/images of the sample. In particular, the series of subsequent readouts/images may be used in order to observe changes in the sample that occur over time. In particular, the series of subsequent readouts/images may be used in order to further increase the number of markers that can be readout or to make sure that the number of markers readout in a single round is not too high in densely labeled samples, i.e. in this case it may be useful to reduce the number of markers to, for example, 1000 markers per round, which is still significantly higher than the methods described in the prior art for fluorescence-based imaging-compatible readout.

Mono-Species Readout Volumes Vs. Multi-Species Readout Volumes

When using the method a spot in the sample, which is defined by the size of the main maximum of the effective point spread function and in the case of confocal microscopy also referred to as the confocal volume, may contain only one marker or a plurality of markers of a single specificity (mono-species readout volume) or may contain markers of multiple specificities (multi-species readout volume). The method presented in this document allows the robust decoding of mono-species readout volumes and provides very high numbers of unique codes. For multi-species readout volumes, however, it cannot be guaranteed that the markers of multiple specificities located in a spot can be decoded, thereby finding only a single possible combination of unique codes, i.e. the decoding of a multi-species readout volume may lead to multiple possible combinations of markers. In the case that a multi-species readout volume is encountered, however, the method can recognize this event reliably and inform the user that a multi-species readout volume was encountered for which an unambiguous solution was not found. The method may further find a limited number of possible alternative solutions and may suggest, based on these solutions, a labelling strategy for at least one further round of staining and imaging with a subset of markers labeled with a new set of fluorescent combinatorial codes. Alternatively or in addition, the user may resort to the approaches described in section “Strategies for densely labeled samples”.

The likelihood to encounter multi-species readout volumes depends on both the number of markers, which are to be read-out in a single round, and also on their subcellular location. A cell has multiple meta-compartments such as the nucleus, the cytoplasm, and the secretory pathway as well as a range of compartments including for example the nuclear membrane, nucleoli (~7%, ~1300 proteins), the nucleoplasm, actin filaments, intermediate filaments, centrosomes, microtubules, the cytosol, mitochondria, the endoplasmic reticulum, the Golgi apparatus, the plasma membrane, secreted proteins, vesicles, which are further divided into sub-compartments. For example, endosomes, lipid droplets, lysosomes, peroxisomes and vesicles are grouped in the cohort of vesicles. While some proteins have a given location in a cell other proteins are multi-localizing proteins (MLPs), which constitute ~55% (n=7106) of the localized proteins in the Cell Atlas (source: https://www.proteinatlas.org/humanproteome/cell/multilocalizing). The number of localized proteins is therefore in the ~14,000 range with ~55% localizing in multiple (sub)compartments. Nucleoli are a particularly dense structure and so far ~7% or 1361 proteins have been detected in one or multiple nucleolar sub compartments: nucleoli (1008), nucleoli fibrillar center (300), nucleoli rim (100) (source: https://www.proteinatlas.org/humanproteome/cell/nucleoli). A typical nucleolus may be in the range of 0.2-3.5 μm in diameter, which means that a small nucleolus of 0.2 μm diameter has a volume of roughly 0.0335 μm³, which is about 1.3-fold larger than the volume of the effective PSF of an NA 1.4 oil immersion objective. For this reason, the nucleolus may be regarded as a challenging structure with respect to multi-species readout volumes. One could estimate that in the worst case ~1500 distinct localized proteins (non-localized proteins excluded) would have to be resolved at the same time in a confocal volume, if one wanted to achieve this in a single round of imaging. Similarly, roughly 24% or 4770 proteins have been detected in the cytosol in humans (source: https://www.proteinatlas.org/humanproteome/cell/cytosol), which consists of ~70% water and 20-30% protein. Cytosolic proteins may be evenly distributed throughout the cytosol or in punctate patterns, like aggresome, cytoplasmic bodies, rods & rings. The odds of encountering a multi-species readout volume in an iterative staining and imaging process can therefore be minimized by taking this a priori knowledge of protein localization into account when defining the sets of markers for each round in a way that they are distributed across the maximally possible number of sub compartments.

### Multi-Species Readout Volume Decoding

Multi-species readout volumes can be reliably detected by the method by acquiring a first readout sequence and retrieving (from a memory device) or computing all combinations of dyes from the plurality of combinations of dyes (S1) subsumable under the first readout sequence. A multi-species readout volume is detected, when more than one combination of dyes from the plurality of combinations of dyes (S1) is subsumable under the first readout sequence. In this case the user is notified, e.g. by a software program, that the corresponding readout volume contains multiple species of target molecules. Depending on possible species in the spot and preferably a priori knowledge from aforementioned protein expression databases an optimized second deterministically assembled set of combinations of dyes from the plurality of combinations of dyes (S1) may be suggested to decode a multi-species readout volume in an iterative decoding process with a minimum number of iterations based on a certain acceptable level of statistical confidence. Alternatively, or in addition, a second independent and identically distributed set of combinations of dyes from the plurality of combinations of dyes (S1) may be assigned to the plurality of affinity reagents in a second round (this might be regarded as random repeated drawing with putting back/replacement).

### Primary Qualitative Iterative Multi-Species Readout Volume Decoding by Reassigning Codes in Between Iterations (“Code Swapping”)

It is important to note though that for a multi-species readout volume that cannot be decoded finding an unambiguous solution in a single round of read-out it is still possible to decode the spot finding an unambiguous solution by multiple rounds of read-out with varying sets of combinatorial combination of dyes attached to the same set of affinity reagents. It is important to note that the cardinality of the set of unique codes can be very high in relation to the cardinality of the set of genes in the human genome or the cardinality of analytes to be identified/to be decoded due to the exponential nature of combinatorial encoding even when a limited amount of dyes is used to generate the codes. For this reason, it is easy to perform experiments in which only a fraction of the available codes are actually assigned to a marker and a target molecule. For example, the number of protein-coding genes in the human genome may be estimated to be roughly 20,000. In an example where n=5 and yA=yB=yC=yD=yE=10, which would generate 100,000 unique codes this means that ~20% of the available codes would actually be assigned. Further, it means that the fraction of actually assigned codes may be easily adjusted over a wide range by adding a further set of dyes or expanding the number of dyes in a set, for example, n=6 and yA=yB=yC=yD=yE=yF=15 which would yield 10,000,000 unique codes and drop the fraction of actually assigned codes needed to encode 20,000 markers to ~0.2%. Importantly 6 excitation lines can be easily provided e.g. on commercial confocal microscopes such as 360 nm, 405 nm, 488 nm, 560 nm, 630 nm, 700 nm and sets containing 15 dyes each of which ~5 each fall into one of three major classes according to their fluorescence lifetime (e.g. <1 ns, 1-5 ns, >10 ns) can be derived from existing fluorescent dyes through modification of the base structure of the dyes. Likewise, several approaches to use fluorescence emission spectral information and lifetime information in conjunction are available and include spectral and fluorescence lifetime, gating, unmixing, phasor-based approaches, machine or deep learning-based classification strategies.

For this reason, it is possible to decode a multi-species readout volume reliably using a particularly preferred embodiment of the method disclosed in this document using an iterative process based on a certain level of statistical confidence such as a p value. A p value measures the probability of obtaining a test result equal to the actually measured value under the assumption that the null hypothesis is true. In the method described in this document a p value for each marker can be calculated that measures the probability that the marker was observed in the readout volume (i.e. the confocal volume or effective point spread function of the readout device) despite the fact that it was not actually present in the readout volume, that the null hypothesis (i.e. the marker is not present in the readout volume) is true. Importantly the confidence in the decoding result grows quickly with each iteration. For this reason, generally acceptable statistical confidence levels, i.e. p values, are attainable with a limited number of iterations such as 1-10.

This is achieved by (1) providing a first set of markers with a first set of affinity reagents labeled with a first set of combination of dyes, which are (randomly or deterministically) selected from the set of combination of dyes, reading out the spot or the readout volume and assembling the first set of all possible combination of dyes subsumable under the first readout sequence, (2) providing a second set of markers with a first set of affinity reagents labeled with a second set of combination of dyes, which are either randomly or deterministically selected from the set of combination of dyes, reading out the spot or the readout volume again and assembling the second set of all possible combination of dyes subsumable under the second readout sequence, (3) comparing the first set of combinations of dyes subsumable under the first readout sequence with the second set of combinations of dyes subsumable under the second readout sequence and removing all combinations of dyes, which are not shared by the first and the second set of combinations of dyes by all possible combination of dyes, i.e. defining the overlap. (4) calculating p values and/or other suitable measures of statistical confidence for each combinations of dyes and/or affinity reagent and/or marker and/or target molecule detected in the overlap. (5) comparing all calculated p values and/or other suitable measures of statistical confidence against a user defined threshold to perform “presence calling” based on a certain level of statistical confidence for at least some of the combinations of dyes and/or affinity reagent and/or marker and/or target molecule detected in the overlap. (6) repeating the process until all multi-species readout volumes of interest have been decoded at a satisfactory level of statistical confidence for at least each combination of dyes and/or affinity reagent and/or marker and/or target molecule in a user-defined subset (e.g. a group of target molecules of interest).

### Secondary Qualitative Multi-Species Readout Volume Decoding

Alternatively or in addition to the method described above, decoding of readout sequences may leverage intensity information. It can be postulated that all dyes exhibit substantially comparable brightness and a substantially linear response in the regime of conditions under which the measurements are performed. Furthermore, differences in the brightness of individual dyes can be accounted for by performing a suitable calibration. This is a general assumption underlying for example fluorescence microscopy measurements. Under this is assumption it can be stated that the likelihood of observing a false-positive result is lower when higher signal intensities are being observed. For example, when a first readout sequence contains a “1” for DyeA.1 and DyeB.2, which means that they were both detected, the intensity of these dyes may be different for example DyeA.1 may have an intensity of 1 AU and DyeB.2 of 10 AU. In this case, codes subsumable under the readout sequence that have a “1” in position DyeB.2 correspond to markers that have a higher probability of being actually present in the readout volume, i.e. better intensity-adjusted pI values and/or other suitable measures of statistical confidence.

### Secondary Quantitative Multi-Species Readout Volume Decoding

Alternatively, or in addition to primary and secondary qualitative decoding a multi-species readout volume may also be quantitatively decoded. This may be brought about by finding the scaling of the proportions of markers in the overlap such that they match the observed intensity profile in the best possible way. As both the intensity profile as well as the identities of the markers in the readout spot are known after primary and/or secondary qualitative decoding (based on a certain level of statistical confidence) this becomes a fully determined set of linear equations, which is essentially comparable to linear unmixing. Using the aforementioned steps multi-species readout volumes can be decoded reliably using a limited number of rounds/iterations providing the identity of markers in the readout spot/volume (i.e. confocal volume/effective PSF) based on suitable measures of statistical confidence and attaining a certain level of statistical confidence, which can be provided in the form of for example marker-specific p values or intensity-adjusted pI values, or on other suitable combinations of dyes-/affinity reagent-/marker-/target molecule-specific measures of statistical confidence. Furthermore, relative quantitative or absolute quantitative information may be derived as well. In this case the response of the readout device and the dye have to be in the linear regime. For an absolute quantitative readout suitable calibrations have to be performed to relate the area under the curve (AUC) for a given dye emission spectrum back to the number of dye molecules. It is important to note that the method does not require an absolute quantitative readout.

### Likelihood of Observing the Same Non-Present Marker Multiple Times

In case the method presented in this document, is used for samples labeled with very high densities such as genome-wide labeling (e.g. 20,000 markers) multi-species readout volumes may occur at high frequency and each multi-species readout volume may contain a high number of species, i.e. different target molecules (e.g. 100-1000 different target molecule species). This leads to the question how high is the likelihood that the same non-present marker is detected multiple times in an iterative decoding scheme. In an example with n=5 and yA=yB=yC=yD=yE=10, □=20,000 markers and □=100,000 available unique codes. How high is the probability to observe a non-present marker in the overlap, i.e. to obtain the same wrong or untrue result, multiple times. In this sense it is useful to consider the probability after the second round. Following to obtaining the first readout sequence the first set of combination of dyes subsumable under the first readout sequence with □□ codes that can be subsumed under the first readout sequence are retrieved from the memory. In our example □□ maybe equal to 1 for a mono-species readout volume or between 1 and 20,000, the maximum number of used codes, for multi-species readout volumes. Generally, it can be expected, however, that for multi-species readout volumes □□ may be in the range of 10-5000. After the round of decoding, therefore significant uncertainty exists with respect to the true content of a multi-species readout volume. This leads to the question: How high is the probability p□ to obtain a readout sequence under which □□ codes can be subsumed? As markers and codes are assigned to each other in a random fashion, it can be postulated amongst all sets of subsumable codes is a stable overlap, i.e. corresponding to the actually present markers in the sample, plus a random assortment of markers, whose codes can by chance be subsumed under the observed readout sequence. The probability p□ that a given marker out of □=20,000 markers is subsumable under the readout sequence, depends on the readout sequence, and is provided by □□□, wherein the □□ is the cardinality of the plurality of combinations of dyes (S1). For example, for, □=1,000 in each round the probability to observe the same non-present marker twice is 1,000:100,000×1,000:100,000 or 1:10,000. Importantly, the likelihood of observing a certain □ can be estimated a priori and used as an information to guide the choices by the user with respect to how many dyes shall be used, i.e. the cardinality of the set of unique codes, how many iterations would be needed to decode a certain number of markers at a certain level of statistical confidence. Whether a higher or lower □□ is observed depends on a number of parameters. For example □□ the assignment rate □=□□□, which corresponds to relation between the set of combination of dyes assigned to a marker to the overall number of combination of dyes available. Further □□ depends on the entropy S in the set of combination of dyes assigned to a marker and is inverse proportional to S, □□□□□S, i.e. a higher the entropy in the set combination of dyes assigned to a marker allows better p values to be obtained in fewer iterations.

In this particularly preferred embodiment the method, which is based on an iterative process of staining the sample, reading out the sample, and inactivating the dyes, further comprises a step of deactivating at least one of the plurality of markers, at least one set of markers, at least one marker. In this document, deactivating one or more markers means preventing the associated fluorescent dye from emitting fluorescence light from the sample in the future. This can be done by either removing the fluorescent dye from the sample or by bleaching the fluorescent dye. Thereby, crosstalk between fluorescent dyes associated with different sets of markers is greatly reduced. In other words, by deactivating a set of markers, the structure marked by said set will not be visible in future images/readouts. This means, for example, that fluorescent dyes with similar emission spectra may be used in subsequent images, thereby, increasing the number of overall markers that can be used in a single round, a single experiment and/or with a single biological sample.

Preferably, the deactivating step is done by at least one of bleaching the fluorescent dye unique to the at least one marker and removing the at least one marker from the sample, preferably by at least one of dissociating or cleaving the fluorescent dye from the affinity reagent or dissociating the affinity reagent from the target structure.

### Discerning Dyes Based on Different Properties

In another preferred embodiment the step of generating the channels is based on at least one of spectral unmixing (which may also be referred to as spectral imaging and linear unmixing, or channel unmixing), a fluorescence lifetime of the fluorescent dyes and an excitation fingerprint of the fluorescent dyes. Spectral unmixing may be performed in various ways including but not limited to linear unmixing, principle component analysis, learning unsupervised means of spectra, support vector machines, neural networks, (spectral) phasor approach, and Monte Carlo unmixing algorithm. In order to reduce crosstalk between the fluorescent dyes associated with different markers, several techniques may be employed. The unmixing techniques are used to separate contributions from different fluorescent dyes to the same detection channel, i.e. the crosstalk due to overlapping emission spectra. Employing these techniques can greatly enhance the sensitivity of the method due to reduced noise. Further, the fluorescence lifetime and the excitation fingerprint of a fluorescent dye can be used in order to correctly identify the fluorescent dye. Phasor S-FLIM for example (as described in Scipioni, L., Rossetta, A., Tedeschi, G. et al. Phasor S-FLIM: a new paradigm for fast and robust spectral fluorescence lifetime imaging. Nat Methods 18, 542-550 (2021)) is a suitable approach to leverage both the emission spectrum and fluorescence lifetime information to increase the overall number of dyes that can be reliably discerned. This can be used to employ more sets of markers per image, i.e. have more sets of markers in one set. In turn, this vastly increases the overall number of markers that can be imaged. Both □ gating and □ unmixing are suitable strategies to take advantage of fluorescence lifetime to increase the number of discernible dyes y per set.

Today fluorescence lifetime is not widely used as an orthogonal contrast in microscopy and cytometry. This is probably related to the fact that most organic dyes, which account for the vast majority of commercially available fluorescent dyes, have fluorescence lifetimes in the 1-5 ns range, which renders the lifetime-based separation challenging. Further and maybe more importantly fluorescence lifetime is strongly dependent on the molecular environment and many dyes show a shortening of fluorescence lifetime in aqueous or polar environments, which are typical for biological specimens. Nevertheless, the widespread use of fluorescence lifetime as an orthogonal contrast seems feasible. In a preferred embodiment of this invention, fluorescence lifetimes of the dyes are stabilized against the environmental conditions by means of one of the following encapsulation, caging, dyad formation, deriving rotaxanes from dyes, co-crystallizing dyes into for example SMILEs, polymerizing dyes, and incorporating dyes into nano- or microstructures such as polymer beads.

In another preferred embodiment machine learning, deep learning or other artificial intelligence approaches are used to train a classifier to discern dyes based on a combination of at least two of the following properties: excitation fingerprint, fluorescence emission spectrum, fluorescence lifetime, fluorescence intensity, brightness. Such a trainable classifier may be similar to “Learning Unsupervised Means of Spectra” (LUMOS) described in McRae T D, Oleksyn D, Miller J, Gao Y-R (2019) Robust blind spectral unmixing for fluorescence microscopy using unsupervised learning. PLoS ONE 14(12): e0225410, which is based on k-means clustering.

In a particularly preferred embodiment, a learning algorithm based on machine learning, deep learning, or artificial intelligence techniques including but not limited to support vector machines, classic neural networks, convolutional neural networks, recurrent neural networks, generative adversarial networks, self-organizing maps, Boltzmann Machines, deep reinforcement learning, autoencoders are trained to separate dyes based on either their emission spectrum.

In a particularly preferred embodiment, a learning algorithm based on machine learning, deep learning, or artificial intelligence techniques including but not limited to support vector machines, classic neural networks, convolutional neural networks, recurrent neural networks, generative adversarial networks, self-organizing maps, Boltzmann Machines, deep reinforcement learning, or autoencoders are trained to separate dyes based on either their emission spectrum and fluorescence lifetime. This may be based on simple fluorescence lifetime gating or more sophisticated fluorescence lifetime analysis. A particularly suitable means to derive training data for this approach may be Phasor S-FLIM for example (as described in Scipioni, L., Rossetta, A., Tedeschi, G. et al. Phasor S-FLIM: a new paradigm for fast and robust spectral fluorescence lifetime imaging. Nat Methods 18, 542-550 (2021)).

In another preferred embodiment the method further comprises a step of capturing a hyperspectral image of the sample. In contrast to multispectral imaging, which captures a limited number of wavelength bands, typically less than or around 10, a hyperspectral image captures tens or hundreds of wavelength bands per pixel. In other words, hyperspectral images have a very high spectral resolution. This allows for a much finer differentiation of fluorescent dyes based on their emission spectrum and thereby increases the sensitivity and reliability of the method.

### Robustness of the Readout

The robustness of the readout is an important consideration. Ideally each spot is readout individually, which means that spots that are readout in parallel are separated spatially such that the optical system used for their detection can resolve them as separate spots. If two or more markers with distinct reactivities/specificities are in too close spatial proximity, i.e. both located substantially within the same readout volume, and are readout simultaneously, then it may happen that an unambiguous decoding of the encoded information cannot be obtained in a single round. In this case, strategies for densely labelled samples may be employed as described below. If densely labelled samples are to be used with very high number of markers such as genome-wide studies, in which multi-species readout volumes may occur with high frequency, the method can be adapted to reliably detect multi-species readout volumes and decode the contained information, i.e. the identity of the markers in the spot, in an iterative process with a limited number of rounds. This is a particularly preferred embodiment of the invention and a breakthrough with respect to the prior art in terms of the ‘plexing’ level that can be attained per round, which is several orders of magnitude higher than currently available methods. This is described above and referred to as “Primary qualitative iterative multi-species readout volume decoding by reassigning codes in between iterations” or “(“code swapping”)”. Further as described above it is also possible to perform relative and absolute quantitative decoding/decryption.

### Strategies for Densely Labelled Samples

When the method according to embodiments of the invention is intended to be used in conjunction with densely labelled samples, it may be beneficial to adapt the method. One such adaptation is based on a priori knowledge of protein location such nuclear, cytoplasmic, nucleocytoplasmic, secreted proteins, proteins located on or in organelles, or on the cell membrane both intracellular and extracellular, which allows the stratification of the plurality of markers into multiple sub-pluralities that are then brought into the sample and acquired in multiple rounds of an iterative staining and imaging process in a way that minimizes the chances of two distinct markers colocalizing in the same spot in the same round. This may be combined with expansion microscopy protocols as described by Martinez et al., Scientific Reports, Volume 10, Article number: 2917 (2020) to expand the sample by a roughly a factor of 4 in all room directions and thereby further reduces the odds of two distinct markers colocalizing in the same spot or readout volume in the same round. Further the number of sets n can be increased, and the plurality of dyes may be divided into sub-pluralities of dyes, each sub-plurality of dyes may then be used to generate combinations of dyes for respective sub-pluralities of markers. As more and more dyes become available with narrower excitation and emission spectra, it will be easier to accommodate a higher n and/or a higher y. A similar problem exists in super resolution microscopy; stochastic labelling of the target structure or stochastic blinking of fluorescent dyes may be used to avoid said problem. Blinking of fluorescent dyes can be achieved in various ways, while some dyes such as for example quantum dots generally blink, other fluorophores may be photoactivated, photoswitched, or ground state depleted for example to make them blink. These techniques can be adapted for the method disclosed in this document to allow the imaging of densely labelled samples. In a particularly preferred embodiment, DNA-PAINT is used and markers are readout stochastically such that an 1 to n readout is reiterated for i times to obtain a first readout sequence. In a preferred embodiment, stochastic optical reconstruction microscopy or a related blinking method is used and markers are readout stochastically such that an A to n readout, is reiterated for i times to obtain a first readout sequence.

### Spot Detection

In order to readout the combination of dyes, it is preferable in some embodiments of the present invention, such as in whole secretome profiling, to ensure that markers of a given specificity are located in disparate locations or spots in the sample and in this case it is useful to perform a spot detection. Spot detection is based on image segmentation. A spot is a kind of feature in the sense of this document. Combination of dyes are preferably readout on a per spot basis in assay formats in which the majority of readout volumes are mono-species readout volumes.

The image segmentation analysis can be carried out with classical approaches, artificial intelligence based techniques including machine learning and neural-networks/deep learning, or other techniques including thresholding techniques, dimensionality reduction techniques, clustering methods, compression-based methods, histogram-based methods, edge detection, dual clustering method, region-growing methods, partial differentiation equation-based methods, variational methods, graph partitioning methods (for example Markov random fields), a watershed transformation, model-based segmentation, multi-scale segmentation, semi-automatic segmentation, trainable segmentation using various machine learning, neural network and artificial intelligence approaches for example pulse-coupled neural networks (PCNNs), and convolutional neural network (U-Net), recurring neural networks (RNNs) as well as object co-segmentation methods such as Markov networks, convolutional neural networks, or long short-term memory (LSTM), for example. Alternatively, or in addition, characteristics such as size and/or colour and/or fluorescent intensity and/or fluorescent lifetime can be used to identify the constituent parts of the marker from the image data. Various algorithms can be used for identification including Harris Corner, scale invariant feature transform (SIFT), speeded up robust feature (SURF), features from accelerated segment test (FAST), and oriented FAST and rotated BRIEF (ORB) are known and can be used to identify the constituent parts and/or features of the marker from the image data.

### General Considerations for Applying the Method

In another preferred embodiment the method further comprises a step of applying the second excitation light temporally after the first excitation light. Preferably, the time between applying the first excitation light and applying the second excitation light is longer than the fluorescence lifetime of the fluorescent dyes of the first set which are excited by the first excitation light. This ensures, that only fluorescence light emitted by the fluorescent dyes of the second set which are excited by the second excitation light is captured for generating the second image/readout. Thereby, crosstalk between fluorescent dyes can be reduced and the sensitivity of the method is further improved.

In another preferred embodiment at least one of the first wavelength spectrum and the second wavelength spectrum for dye excitation comprise a wavelength range being smaller than 50 nm, smaller than 30 nm, smaller than 10 nm or a single wavelength. These wavelength bands are typical ranges of e.g. dichroitic beam splitters or bandpass filters being used in fluorescence microscopy. Various methods can be used in order to generate the respective wavelength spectrum for sample illumination or fluorescent dye excitation. For example, a bandpass filter which filters out a wavelength range might be used in combination with a light source emitting light having a broad spectrum of wavelengths, e.g. a mercury or xenon lamp. Alternatively, or additionally, a white light laser emitting supercontinuum white light in combination with an AOTF for selecting of one or more single wavelengths of the emitted light could be used.

In another preferred embodiment the fluorescent dyes unique to each set can be excited by essentially one wavelength spectrum or by the same wavelength spectrum. This allows the fluorescent dyes of a single set to be excited by a single light source with e.g. a narrow emission spectrum. This embodiment of the method can be easily implemented with existing fluorescence microscopes which often comprise such light sources.

In another preferred embodiment the fluorescent dyes of a single set of dyes A to n comprise emission spectra of at least partially different wavelength ranges. Thereby, the fluorescent dyes of a single set of dyes A to n can be easily distinguished from another by their emission spectra. This reduces or eliminates the computational load of the unmixing necessary to separate the channels of each image and makes the method faster and more reliable.

In another preferred embodiment at least two fluorescent dyes, each unique to one set of dyes A to n, have different fluorescent lifetimes. Thereby, the at least two fluorescent dyes can be distinguished from another by their lifetimes. In particular, this can be used to increase the number of channels per image, i.e. capture more markers per image. Thus, the overall number of dyes per set that can be imaged is vastly increased. In particular, existing fluorescent dyes may be engineered to generate derivative fluorescent dyes with similar excitation and/or emission spectra, but different fluorescence lifetimes by modifying the base structure or putting the fluorescent dye into a different molecular environment. In particular, existing fluorescent dyes may encapsulated in micro- or nanocapsules, embedded into a polymer like polystyrene, caged, or co-crystallized in for example SMILES to stabilize their molecular environment and thereby their fluorescence lifetime. This strategy may also be used to generate sets of dyes of the same dye species with different fluorescence lifetimes. Rotaxanes and in particular rotaxanes derived from squaraine are interesting dyes in this regard, as the interlocking of the dye molecule in a macrocycle stabilizes the molecular environment and thereby the fluorescence lifetime.

In a particularly preferred embodiment at least one of the dyes or labels is a small-molecule ionic isolation lattices (SMILES), these are small crystals that consist of cationic dyes which are co-crystallized with counterions such as for example anion-binding cyanostar or alternative agents, such as Bis-amide, cyclodextrin, Tetra-phenyl or pyrene as described in Benson et al. 2020 Chem 6, 1978-1997, Aug. 6, 2020.

In a particularly preferred embodiment at least one of the dyes or labels is a polymer microbead or nanostructure containing a small-molecule ionic isolation lattices (SMILES).

In a preferred embodiment at least one of the dyes or labels is a rotaxane dye such as squaraine-rotaxane dyes.

In a preferred embodiment at least one of the dyes or labels is a dyad consisting of an antenna moiety and emitter moiety.

In a particularly preferred embodiment at least one of the dyes or labels is a FRET pair of at least two dyes a donor and an acceptor connected by a linker, such as a nucleic. The at least one of the fluorescent dyes may be a FRET-pair based having at least one fluorescent dye as FRET donor and at least one fluorescent dyes as a FRET acceptor. The FRET-pair might be physically connected by a linker comprising DNA, RNA, peptide nucleic acid, morpholino or locked nucleic acid, glycol nucleic acid, threose nucleic acid, hexitol nucleic acid or another form of artificial nucleic acid, a DNA nanostructure and or a peptide.

In another preferred embodiment at least two fluorescent dyes, each unique to one marker, in the first and/or second sub-pluralities comprise emission spectra of essentially the same wavelength ranges and essentially the same fluorescent life time at a first condition such as a certain first pH value, a certain first solvent, a certain first redox level, certain first temperature, or a certain first concentration of a ligand (e.g. lower concentration of one of the following Cu(II), Zn(II), a small molecule) of the sample and comprise emission spectra of essentially the same wavelength ranges and substantially different fluorescent life time at a second condition such as a certain second pH value, a certain second solvent, a certain second redox level, certain second temperature, or a certain second concentration of a ligand (e.g. lower concentration of one of the following Cu(II), Zn(II), a small molecule) of the sample.

Embodiments of the invention also relate to a device for analyzing a biological sample being adapted to carry out the method for analyzing a biological sample describe above. The device has the same advantages as the method and can be supplemented using the features of the dependent claims directed at the method. The device may in particular be configured to image samples in an array format such as a microplate.

In a preferred embodiment the device is configured to readout samples flowing through a flow cell.

In a preferred embodiment the device comprises at least one of a first light source configured to emit the first excitation light, and at least one second light source configured to emit the second excitation light. Alternatively, or additionally, the device comprises a tunable light source configured to emit the first and second excitation light. Preferably at least one of the first excitation light and the second excitation light is coherent light.

In another preferred embodiment a separation of the first and/or second images (readouts) into the at least two channels is done by at least one of a spectrometer comprising a prism or a grating and at least one detector. Diffractive elements can be used to optically or spatially separate the captured fluorescence light by wavelength into distinct channels, e.g. by directing different wavelength onto different parts of a single detector or onto different detectors. Since these channels are created by detector hardware they will also be called detection channels in the following. An example for such a spectrometer arrangement for a confocal scanning microscope is disclosed e.g. in U.S. Pat. No. 6,614,526 B1.

In another preferred embodiment a separation of the first and/or second images (readouts) into the at least two channels is done by at least one time-sensitive detector. Such detectors register not only the wavelength spectrum but also the arrival time of the captured fluorescence light. They may also be time-gated, i.e. configured to register events within discrete segments of time so called time gates, enabling e.g. the determination of lifetime information from the arrival time of the captured fluorescence light. Thereby, fluorescent dyes having significantly overlapping emission spectra but different fluorescence lifetimes can be separated reliably into different channels. This further increases the number of markers that can be grouped into a single sub-plurality, i.e. imaged at the same time.

Embodiments of the invention further relate to a microscope system comprising the device for analyzing a biological sample described above. The microscope system is preferably a lens-free microscope, a light field microscope, widefield microscope, a fluorescence widefield microscope, a light sheet microscope, a scanning microscope, or a confocal scanning microscope.

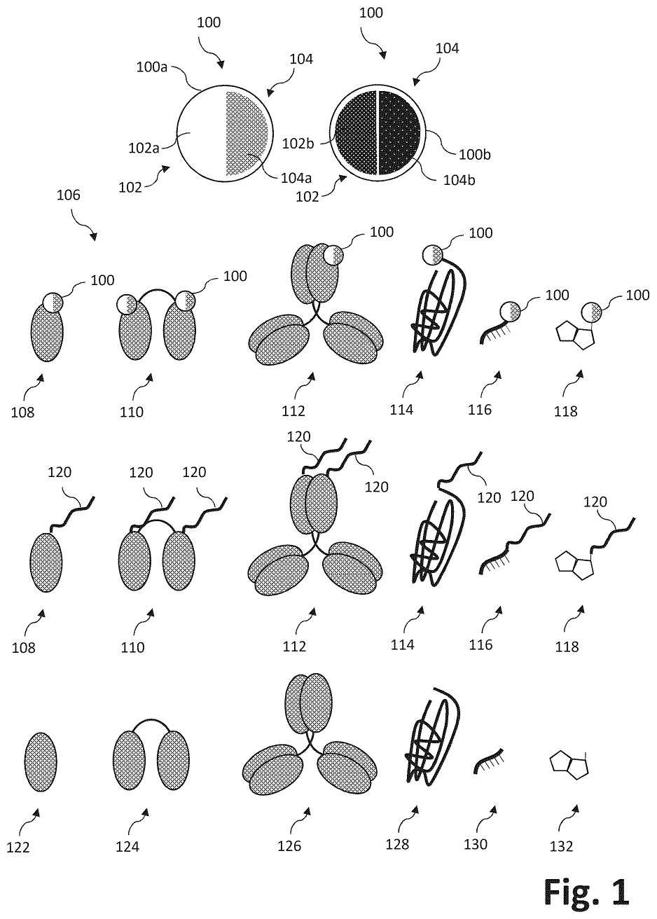

FIG. 1 schematically shows a prior art fluorescent dye which may also be named a label 100 represented as a circle, wherein the left half 102 is filled by a pattern indicating/reflecting the excitation spectrum, such that dyes with the same left half pattern are excitable with the same excitation light, wherein the right half 104 is filled by a pattern indicating/reflecting all properties that may be used individually or in conjunction to discern dyes from each other by a device used to perform the readout, which includes for example the fluorescence emission spectrum, fluorescence lifetime, fluorescence polarization, brightness, and excitation fingerprint, such that two dyes depicted with the same right half cannot be discerned by the device used for readout. FIG. 1 shows two fluorescent dyes 100a, 100b. Each fluorescent dye is pictured as a circle 100a, 100b with a solid border. Each circle 100a, 100b is divided into two half circles 102a, 102b, 104a, 104b with different hatching. The type of hatching of the left half circles 102a, 102b indicates the excitation light the respective fluorescent dye 100a, 100b can be excited with, i.e. fluorescent dyes having left half circles 102 with the same type of hatching can be excited with excitation light having the same wavelength spectrum or same single wavelength. The type of hatching of the right half circles 104 indicates the properties of the respective fluorescent dye 100 that can be used for their discrimination on the imaging system used for the data acquisition step or during the readout, including for example their emission spectra, their fluorescence lifetime, excitation fingerprint. This means, fluorescent dyes 100 having right half circles 104 with the same type of hatching have the same or essentially same, i.e. indistinguishable emission characteristics.

It is important to note that this is conceptually dependent on the readout as a readout that provides orthogonal contrasts such as emission spectrum and fluorescence lifetime for instance can differentiate more dyes as compared to readouts that provide only one kind of contrast. FIG. 1 further shows a range of commonly used affinity reagents 106, including single-domain antibodies 108, multimerized (single-domain) antibodies 110, conventional antibodies 112, aptamers 114, oligonucleotides 116, toxins/drugs/drug-like molecules/small molecules (e.g. biotin) 118, which are labeled by direct conjugation to a dye 100. The same affinity reagents may also be tagged with peptide tags, haptens or oligonucleotides. FIG. 1 further shows a range of commonly used affinity reagents 106, including single-domain antibodies 108, multimerized (single-domain) antibodies 110, conventional antibodies 112, aptamers 114, oligonucleotides 116, toxins/drugs/drug-like molecules/small molecules (e.g. biotin) 118, which are labeled by direct conjugation to an oligonucleotide 120.

In the sense of this document 124 shall represent not only dimers but multimerized (single-domain) antibodies in general. Such combinations 124 may be engineered in order to achieve specific affinities that are not obtainable otherwise (bispecific reactivity) or to increase avidity

Direct and indirect immunofluorescence labelling is widely used in life science research and diagnostic application to analyse the presence of molecular targets such as proteins, RNA, DNA, and other molecules. Typically, such fluorescence-based assays are read-out using at least one of a plate reader, a high content screening device, a microscope, a cytometer, or a fluorescence-activated cell sorter.

Read-out devices used to perform fluorescence multicolour imaging typically include at least one light source for generating excitation light, a detection system including at least one detection channel and may as well contain filters and/or dispersive optical elements such as prisms and gratings to route excitation light to the sample and emission from the sample to the detection system and/or onto to appropriate areas of the detector.

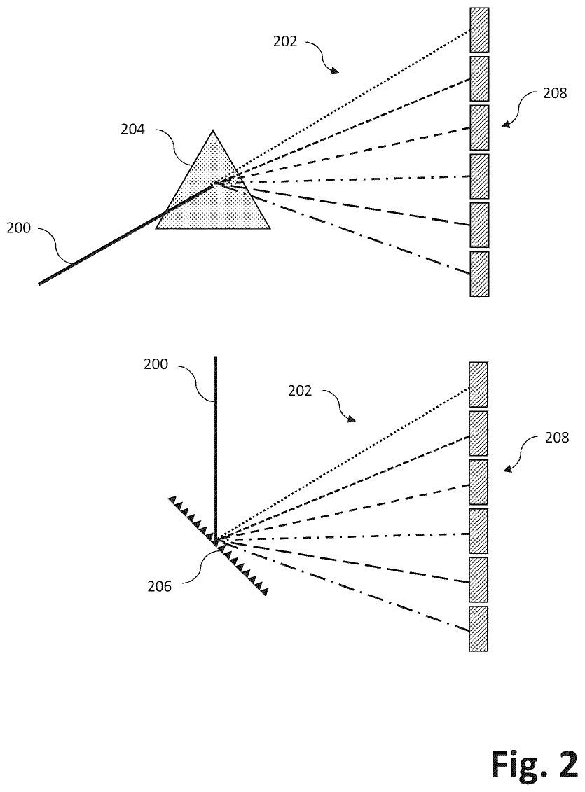

FIG. 2 schematically shows two detection systems with dispersive optical elements (e.g. a prism, or a grating) 204, 206 and a detector with multiple channels 208. The detection system in the sense of this document may consistent of several detection channels, may be a spectral detector detecting multiple bands of the spectrum in parallel, or a hyperspectral detector detecting a contiguous part of the spectrum. The detection system contains at least one detector, which may be a point-detector (e.g. a photomultiplier, an avalanche diode, a hybrid detector), an array-detector, a camera, hyperspectral camera. The detection system may record intensities per channel as is typically the case in cytometers or may be an imaging detection system that records images as in the case of plate readers or microscopes.

Read-out devices allow a certain number of dyes to be analysed from a given biological sample in a given run. This number typically depends on the number of detection channels, n, the read-out device is configured to provide, i.e. able to spectrally resolve, or discern. In the case of microscopes the number of detection channels is typically 4-5 in the case of camera-based widefield detections (e.g. widefield epifluorescence microscopes, spinning disk microscopes, light sheet fluorescence microscopes), 5-12 detection channels in the case of microscopes with spectral detection concepts that typically rely on excitation or emission fingerprinting and (spectral/linear) unmixing. In order to detect the emission from multiple fluorophores, quantum dots, and/or fluorescent proteins and assign detected photons to the corresponding fluorophores, quantum dot, and/or fluorescent protein from which they are emitted read-out devices may employ various strategies. Emission filters are commonly used to direct desired bands of the spectrum to the detector. Multiple emission filters are typically installed on filter wheels such that bands of emission light reaching the detector can be swiftly changed. Alternatively, or in addition dispersive optical elements schematically shown in FIG. 2 may spectrally separate the light 200 emitted from the biological sample. Both prisms 204 and gratings 206 find widespread use in excitation and emission beam routing and spectral separation in read-out devices like microscopes, cytometers, and plate-readers for instance. On the detection side prisms 204 and gratings 206 may be used in simple or more complex arrangements to spectrally separate the emission light 202 and route it onto a suitable detector 208, which may consist of multiple point-detectors (e.g. photomultipliers, avalanche diodes), array detectors, cameras or hyperspectral detectors. Depending on the configuration of the detection system the read-out device will be capable of separating emission from a number n of dyes reliably. In the way, in which read-out devices are currently being used, this number n determines the number of molecular markers, which can be acquired on the respective read-out device per round. Alternatively, or in addition to separating dyes based on their spectral properties it is possible to use fluorescence lifetime □, as a means to separate fluorescence emission on devices which are configured to measure lifetime, i.e. have pulsed laser light source and time-resolved detectors.

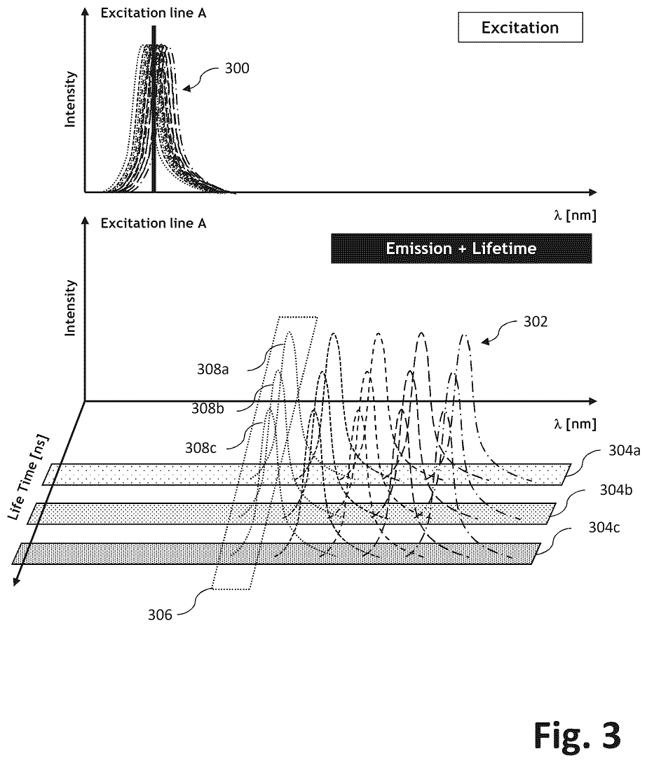

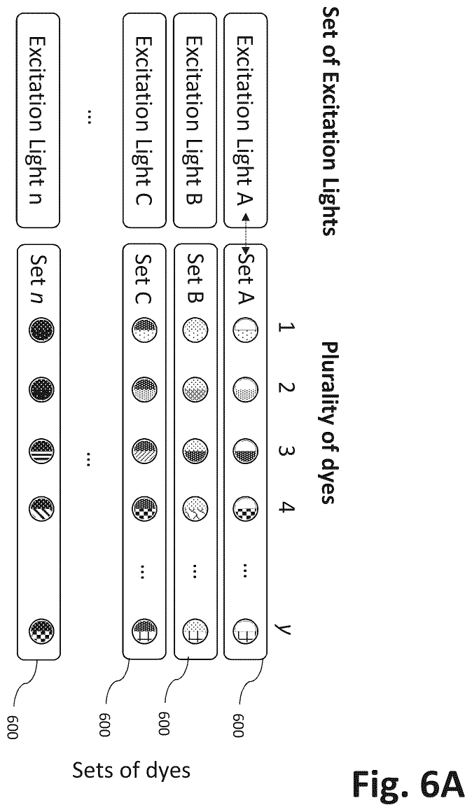

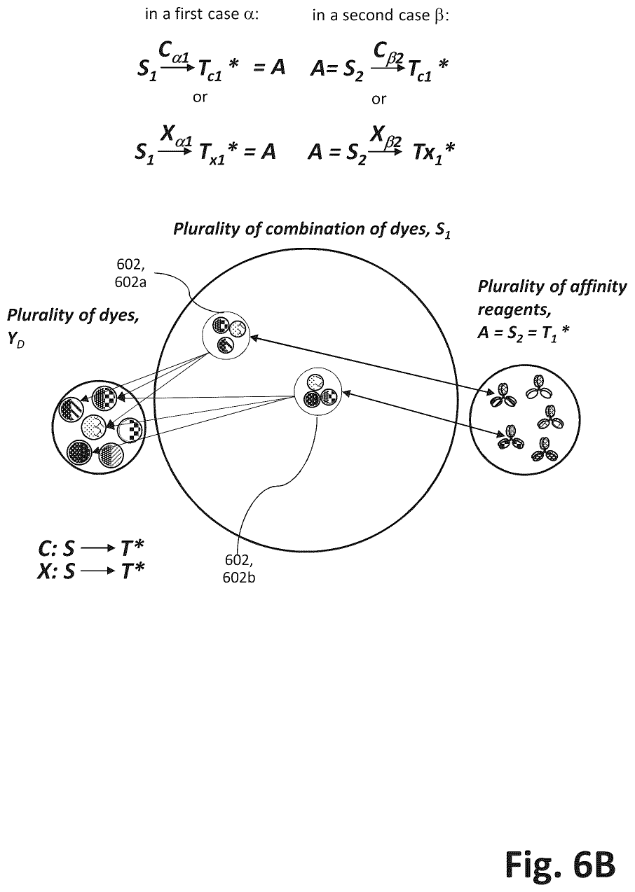

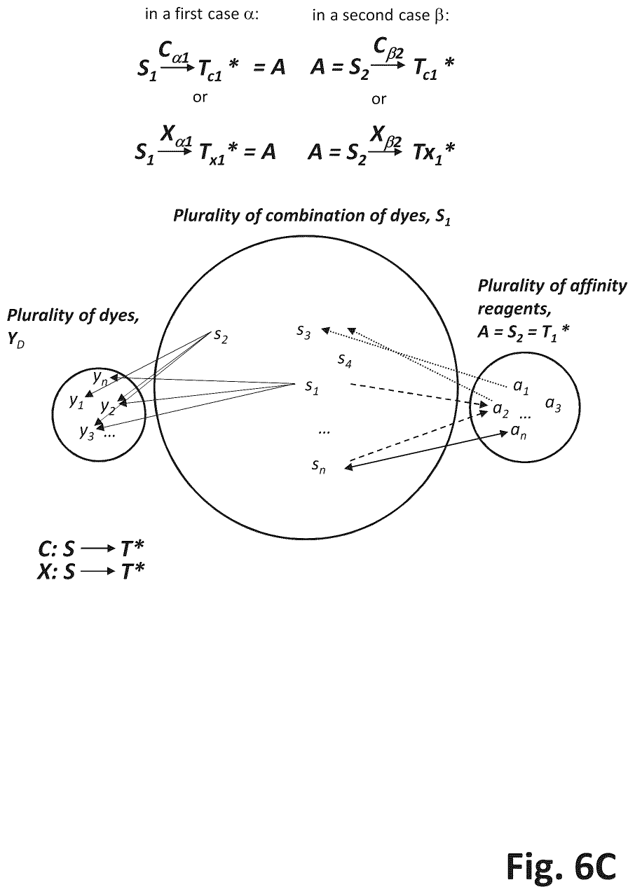

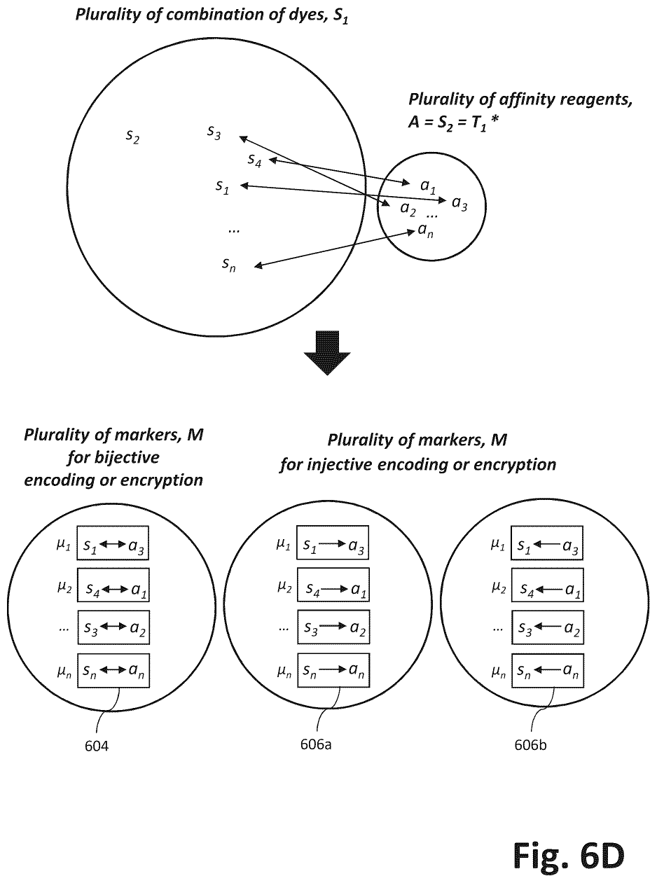

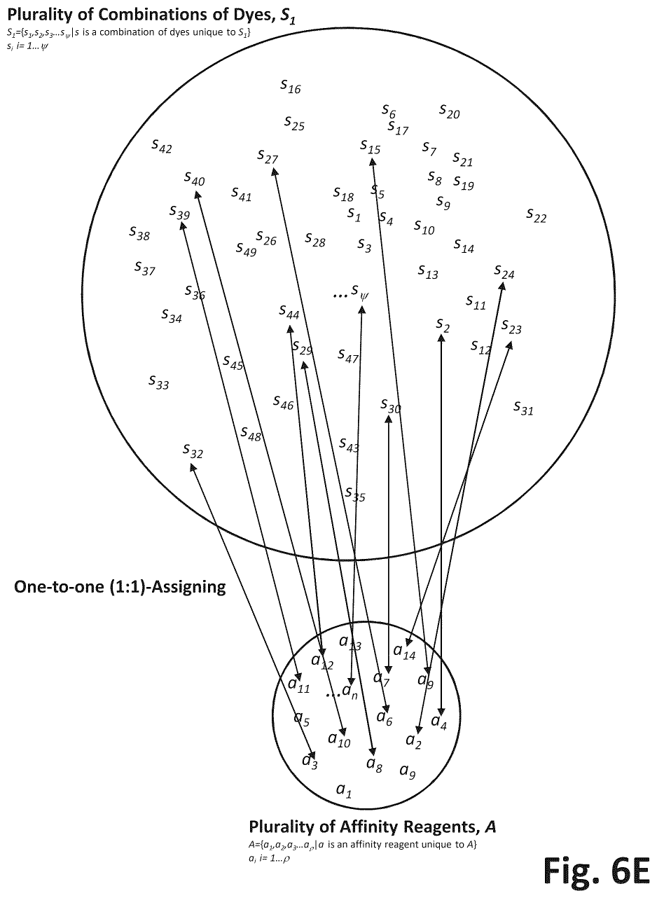

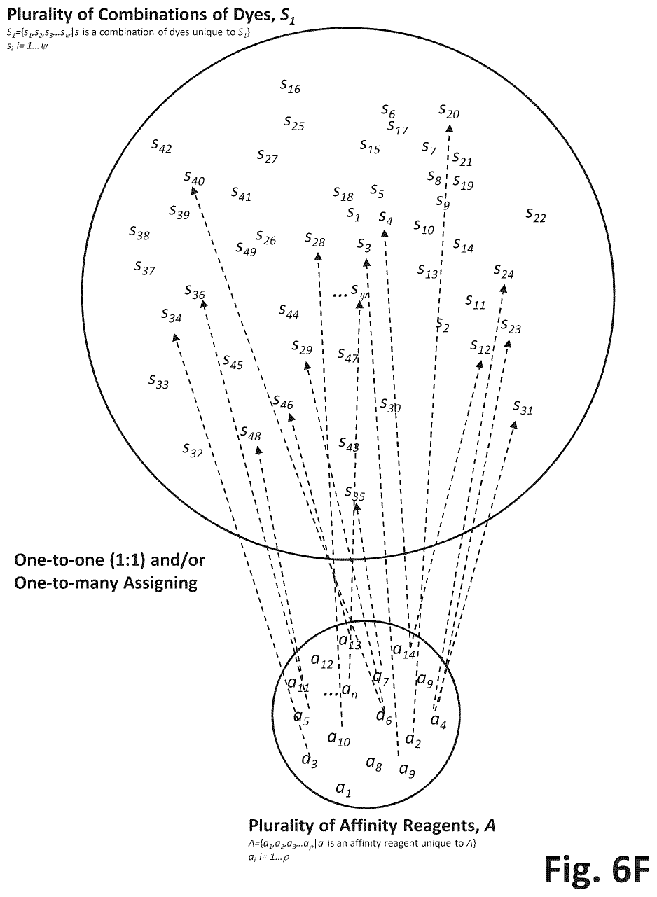

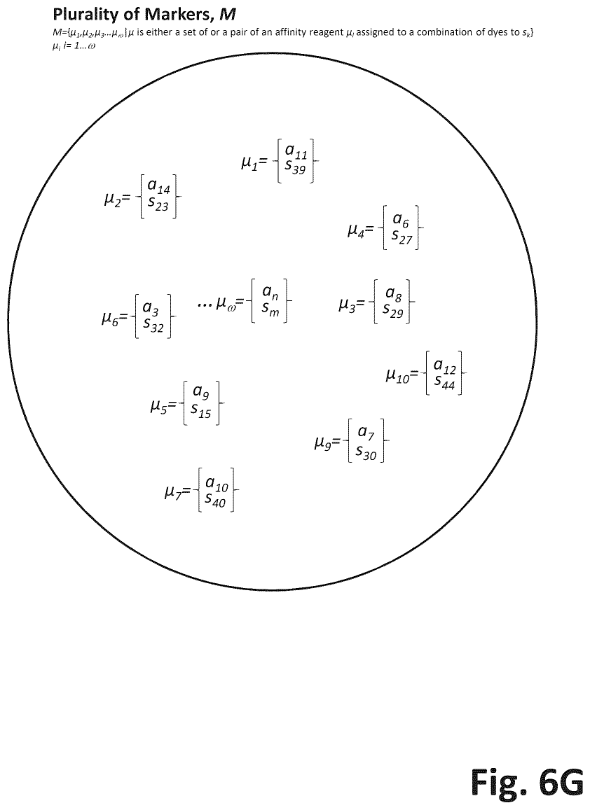

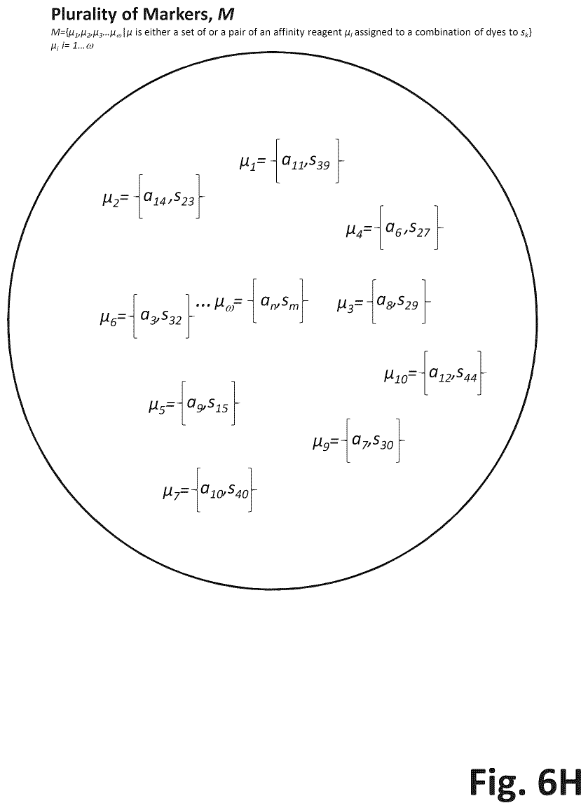

FIG. 3 schematically shows a part of the concept as disclosed in PCT/EP2021/063310 in which the excitation and emission characteristics of one set of fluorescent dyes from a plurality of n sets of fluorescent dyes is schematically shown. As shown in FIG. 6A sets of fluorescent dyes are build such that each dye in a set can be excited with the same excitation light, a single band, a multi-band, a single wavelength, or a plurality of wavelengths. Sets of fluorescent dyes are configured in a way that the dyes can be discerned from each other by the readout device and readout software. This means that readout devices with more channels and particularly readout devices that provide orthogonal contrasts allow for more dyes to be unequivocally assigned to channels. In the example shown in FIG. 3 a set of fluorescent dyes excitable with excitation line A, for example 390 nm contains 15 dyes, which are separated by their fluorescence lifetime into 3 groups or gates (based on r gating or r unmixing) 304a, 304b, 304c, each group holding 5 fluorescent dyes which can be discerned from each other based on their emission spectrum.

FIG. 3 schematically shows a prior art example which leverages the principle of forming n sets of selectively excitable dyes, i.e. dyes from set A are excitable by excitation light A, but substantially not excitable with excitation light B to n, in conjunction with a readout that can differentiate up to y dyes per set or up to yA, yB . . . yn, in case the number of dyes for each set may be different.

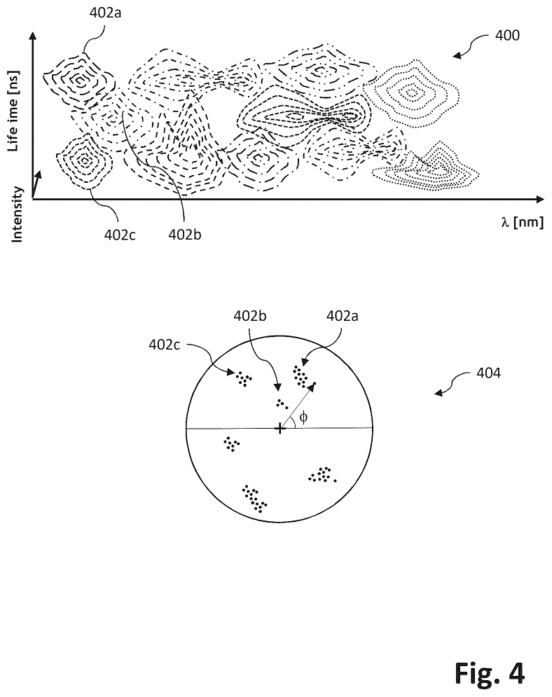

FIG. 4 schematically illustrates a “topographical approaches” 400, in which for example spectrum is plotted against the fluorescence lifetime and intensity, wherein lines of the same pattern correspond to different levels of (normalized) intensity of the same dye species 402a, 402b, 402c. Topographical approaches are particularly suited for machine learning, deep learning or other forms or artificial intelligence-based dye separation, by means of training a classifier. Such training may be supervised or unsupervised. The rich texture that this “topography” provides is ideal input for these approaches. Phasor-based approaches 404 for dye separation using information like intensity and/or emission spectrum and/or fluorescence lifetime are likewise suited both for classical dye separation algorithms or approaches as well as for machine learning, deep learning or other forms or artificial intelligence-based dye separation. Again, such training may be supervised or unsupervised.

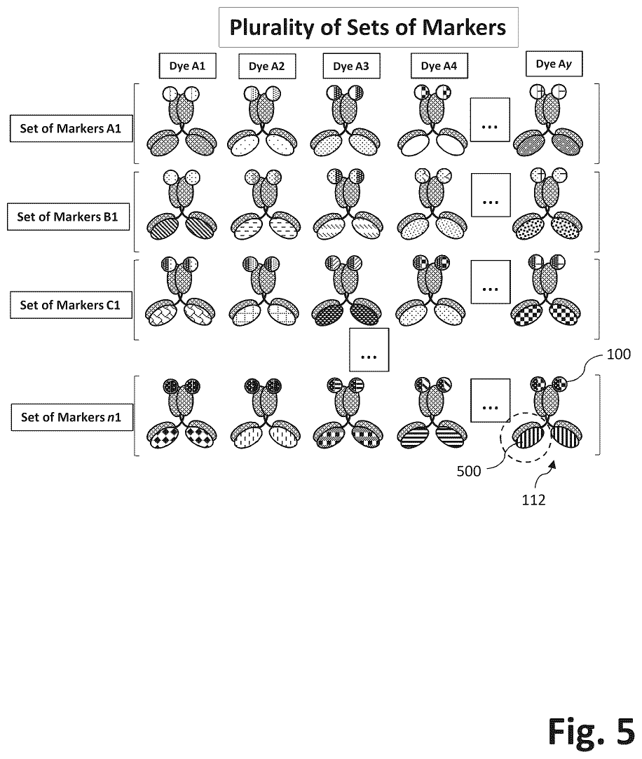

FIG. 5 schematically shows an example known in the art which leverages a 1:1 relationship between the unique specificity of the affinity reagent and a dye unique to the plurality of dyes, which means that a dye 100 corresponds to a channel, which corresponds to a marker in this case. Embodiments of the present invention change this 1:1 relationship between the unique specificity of the affinity reagent and the unique dye and replaces it conceptually with an injective or preferentially a bijective (i.e. 1:1 correspondence) relationship between a marker and a combination of dyes in a single round and a 1:many relationship between a marker and multiple combinations of dyes over multiple rounds. For example, using the approach as described in PCT/EP2021/063310 with a plurality of dyes, wherein n=5 and yA=yB=yC=yD=yE=10, would allow the readout or imaging of 50 dyes 100 and thus 50 markers of 50 different reactivities/specificities 500. This illustrates the strong advantage of the embodiments of the present invention with respect to the number of markers that can be readout in a single round and overall, i.e. in an experiment with a number of iterations which may be acceptable to the majority of users and may be in the range of 1-25 iterations. For example a genome-wide readout (~20,000 proteins corresponding to ~1 molecular target/analyte per protein-coding gene) is possible with embodiments of the present invention using only 2 rounds obtaining p values that may be generally acceptable for the vast majority of users in the vast majority of applications. More importantly, p values can be improved exponentially with every further round rendering method extremely powerful. At its core this is related to the exponential increase of the cardinality of the set of unique codes (plurality of combinations of dyes S1), which is opposed to a high but limited number of markers needed for genome-wide readout.

FIG. 6A shows the plurality of dyes grouped into sets of dyes A to n 600, based on their excitability with the excitation lights A to n, and the yA to yn members of each set. As depicted the plurality of dyes contains yA+yB+yC . . . +yn=y□□ members or elements. The amount of the excitation lights might be n whereas the amount of dyes might have a different value to n as indicated above.

FIGS. 6B and 6C schematically illustrates the relationship between the plurality of dyes YD from which the plurality of combinations of dyes S1 is formed by composing combinations of dyes 602 following a certain “rule of forming combinations of dyes” in a way that each combination of dyes in the plurality of combinations of dyes S1 is unique and that the plurality of combination of dyes S1, preferably, contains a maximum amount of elements (i.e. combinations of dyes), that can be formed by the respective “rule of forming combinations of dyes”, or in other words such that the cardinality of the plurality of combination of dyes S1 □ is maximal, preferably, composing the combinations of dyes in a way that they are unique and randomly distributed. Many different “rules of forming combinations of dyes” are compatible with the method. FIG. 6B further shows the mapping between the plurality of combinations of dyes S1 and the plurality of affinity reagents A=S2=T1* using a code C1, C2, C3, . . . Cn and/or a cipher X1, X2, X3, . . . Xn, i.e. the encoding or encryption. The encoding and/or encryption may be performed in different ways illustrated as case α and case β. In both cases the codes C1, C2, C3, . . . Cn and/or a ciphers X1, X2, X3, . . . Xn used are total functions that are preferably bijective and alternatively injective.

Referring to FIG. 6B:

$$C : S \to {T}^{*}$$

$$X : S \to {T}^{*}$$

C is a code, which is a total function
X is a cipher, which is a total function
S is a source alphabet
T is a target alphabet
T* is a set of sequences of symbols over T
in a first case α.

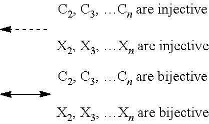

in a second case β:

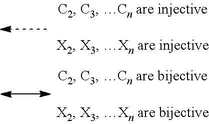

C₁, C₂, C₃, . . . Cₙ are codes
C₁, C₂, C₃, . . . Cₙ are either bijective (preferred embodiment) or injective
C_{α1}, C_{α2}, C_{α3}, . . . C_{αn} are codes 1 to n used in a case α
C_{β1}, C_{β2}, C_{β3}, . . . C_{βn} are codes 1 to n used in a case β
X_{α1}, X_{α2}, X_{α3}, . . . X_{αn} are ciphers 1 to n used in a case α
X_{β1}, X_{β2}, X_{β3}, . . . X_{βn} are codes 1 to n used in a case β

Referring to FIG. 6C:

$$C : S \to {T}^{*}$$

$$X : S \to {T}^{*}$$

C is a code, which is a total function
X is a cipher, which is a total function
S is a source alphabet
T is a target alphabet
T* is a set of sequences of symbols over T
in a first case α:

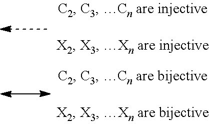

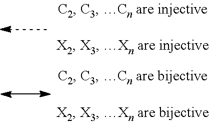

C₁, C₂, C₃, . . . Cₙ are codes
C₁, C₂, C₃, . . . Cₙ are either bijective (preferred embodiment) or injective
C_{α1}, C_{α2}, C_{α3}, . . . C_{αn} are codes 1 to n used in a case α
C_{β1}, C_{β2}, C_{β3}, . . . C_{βn} are codes 1 to n used in a case β
X_{α1}, X_{α2}, X_{α3}, . . . X_{αn} are ciphers 1 to n used in a case α
X_{β1}, X_{β2}, X_{β3}, . . . X_{βn} are codes 1 to n used in a case β

FIG. 6D schematically illustrates the assigning or mapping of affinity reagents to combinations of dyes or vice versa. The illustration on top shows double-headed arrows resembling bijective mapping, which means that pairs ai-si are formed by the encoding/encryption, in other words there is a one-to-one correspondence between an element ai of the plurality of affinity reagents A=S2=T1* and element si from the plurality of combination of dyes S1. The injective case is permitted as well, but not visualized in the illustration for the sake of clarity. Further FIG. 6D shows how the assigning of (encoding/encrypting) an affinity reagent ai of the plurality of affinity reagents A=S2=T1* to a combination of dyes si from the plurality of combination of dyes S1 or vice versa, leads to a marker μi from the plurality of markers M. As long as this assignment is purely virtual a marker may be considered as a virtual marker, when the physical correlate i.e. a certain affinity reagent such as antibody molecules are physically coupled to the correlate of a combination of dyes such as a certain mix of dyes or a certain reporter then, the marker may be regarded as a “physical marker”.

FIG. 6E schematically illustrates a one-to-one (1:1) assigning or mapping of affinity reagents to combinations of dyes or vice versa. A mapping such as this may be described as “bijective”.

FIG. 6F schematically illustrates a one-to-many assigning or mapping of affinity reagents to combinations of dyes. A mapping such as this may be described as “at least injective”.

At least some affinity reagents a from the plurality of affinity reagents A are uniquely assigned to a combination of dyes s from the plurality of combinations of dyes S1. In both mappings, no combination of dyes s is assigned to more than one affinity reagent a from the plurality of affinity reagents A. However, in a one-to-many mapping, an element aj from the plurality of affinity reagents A may be assigned to more than one combination of dyes s from the plurality of combinations of dyes S1. In this case multiple unordered pairs including aj are formed each corresponding to a given marker. For example, the markers μh={aj,sf}, μh′={aj,sz}, μh′={aj,sr} may be three distinct markers that share the same affinity reagent aj (e.g. in the same round or in different rounds). In such a case a given target would be addressed by multiple markers with different combinations of dyes s. Further the same target may be addressed with multiple markers using distinct affinity reagents a from the plurality of affinity reagents A that bind to the same predetermined structure or analyte. Alternatively, or in addition ordered pairs between affinity reagents a from the plurality of affinity reagents A and combinations of dyes s from the plurality of combinations of dyes S1 are formed. Exemplary illustrations of markers forming bijective pairings are shown in FIGS. 6G and 6H. The pairings shown are bijective, or one-to-one, because no affinity reagent or combination of dyes is shared by any two markers.

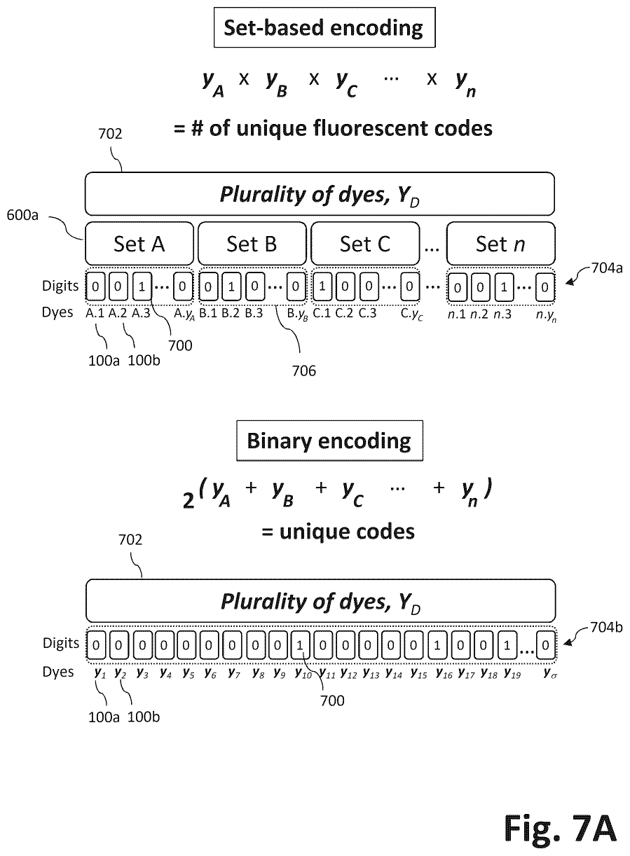

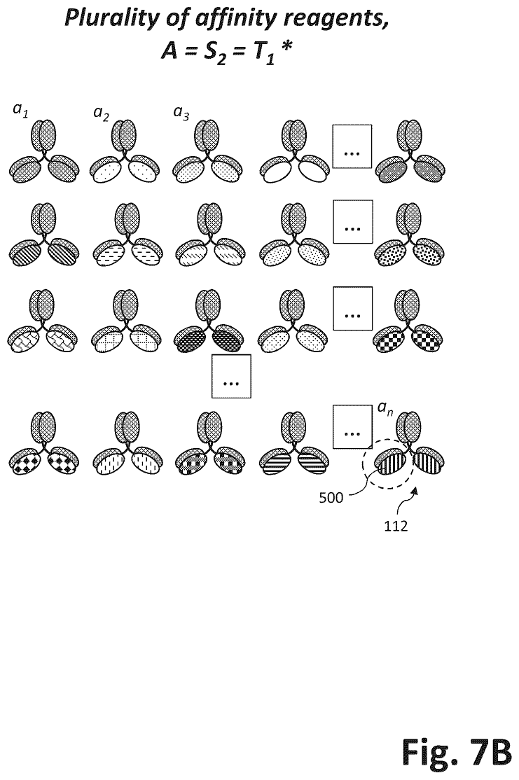

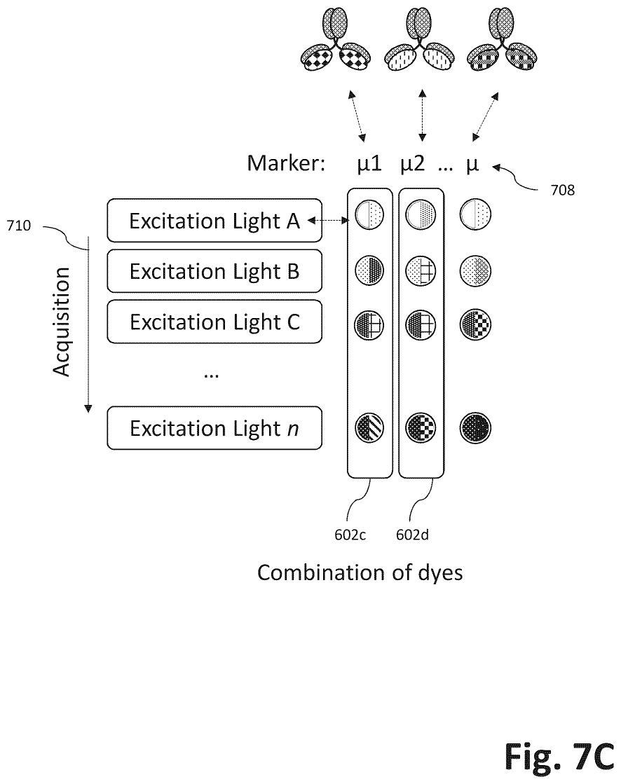

FIG. 7A schematically shows two distinct examples of “rules of forming combinations of dyes” from a plurality of dyes YD, 702. In a particularly preferred embodiment of the invention each dye corresponds to a digit 700 in a binary code 704b, which is a digital code that is structured in code blocks/code segments 706 corresponding to the sets of dyes A to n such that in each code blocks/code segments 706 contains a single “1”, such that each combination of dyes comprises/contains n dyes. In other words, if for example excitation line 1-5 are 405 nm, 488 nm, 560 nm, 630 nm, 700 nm, then in each block corresponding to a set of dyes A to n, the encoding will randomly select one of the dyes in that set to be included in the combination of dyes. For example, 405 nm—Atto930; 488 nm—Atto490 LS; 560 nm—Alexa Fluor 564, 630 nm—Alexa 633 nm, 700 nm—Atto690. In this case the encoding provides yA×yB×yC . . . ×yn combinations of dyes in the plurality of combinations of dyes S1.

“Set-based encoding” and “Binary encoding” are based on certain “rules of forming combinations of dyes”. In “set-based encoding” dyes y from the plurality of dyes YD are grouped into sets A to n excitable with the respective excitation lights A to n. A combination is formed by selecting one dye from each set A to n such that the number of combinations of dyes in the plurality of dyes S1 is maximized and such that each combination of dyes is unique to the plurality of combinations of dyes. In set-based encoding each combination of dyes is an n-tupel with n members. In “binary encoding” the “rule of forming combinations of dyes” is such that each dye from the plurality of dyes y1, y2, y3, . . . y□ corresponds to a particular digit 700 in a binary code. In “binary encoding” according to this preferred embodiment each combination of dyes comprises or contains 1 to y□ members.

In another particularly preferred embodiment of the invention each dye corresponds to a digit 700 such that each code 704b may comprise/contain a variable number of dyes which is randomly selected and between 1 and yA+yB+yC . . . +yn=y□. This is named as “binary encoding” in this document and yields 2 (yA+yB+yC . . . +yn) number of combinations of dyes in the plurality of combinations of dyes S1.

While combinatorial coding has been described in the prior art, the number of codes that could be obtained was limited, as the number of dyes that could be used was limited to around 5. Using the method disclosed in this document it is feasible to define pluralities of dyes with 25 or more members from commercially available fluorescent dyes that can be readout using commercially available readout devices. Using available detector and dye technology it is feasible based on the method disclosed herein in to define pluralities of dyes with 120 or more members from available fluorescent dyes that can be readout using dedicated readout devices. This is feasible, when for example n=8 (e.g. excitation line 1-8: 360 nm, 405 nm, 440 nm, 488 nm, 560 nm, 590 nm, 630 nm, 700 nm) excitation lines are used to with sets of yA=yB=yC . . . =yn= 15 dyes, which are grouped into 3 □ classes, such that each □ class holds 5 dyes, which are sufficiently spectrally separated. In this case the cardinality, i.e. the number of combinations of dyes that can be encoded in the set of combinations of dyes is in the range of 2.56 billion combinations of dyes for the set-based encoding and on the order of 1.33×1036 in the case of binary encoding. While this number is significantly higher than, for example 20,000 to 30,000, which is a rough estimate for the number of protein-coding genes in the human genome, it may still be preferable to work with such high numbers of available codes as this allows setting up experiments in a way that only a small percentage would actually be used. If a n=8 and y=15 set would be used with this method to analyze 20,000 target molecules □ would be around only 0.00078% of the available codes in the case 2.56 billion combinations of dyes in our example for set-based encoding. If for the same example binary encoding would be used then □ the fraction of combinations of dyes actually assigned to a marker from the set of unique codes would be in the range of 0.000000000000000000000000000002%. It is important to note that this is a situation which is already attainable based on existing dyes and readout technology. Working with very small □ means that, the entropy of a set of randomly assigned combinations of dyes will be higher, and consequently the decoding will be easier. A small □□ may also lead to a lower probability of observing type II false-positives a number of times.

In a particularly preferred embodiment of the invention this strategy is used to improve the readout of spots in densely labeled samples

FIG. 7B schematically shows an example of the plurality of affinity reagents.

FIG. 7C schematically shows how a given readout volume in the sample, which for example contains a single target molecule bound by a marker 708, which comprises a reporter, which comprises a linker and a combinations of dyes 602c, d that is readout in a single 1 to n acquisition sequence 710. The n=5 sets of dyes may for example be excited at 405 nm (set A), 488 nm (set B), 565 nm (set C), 630 nm (set D), at 700 nm (set E) or in a reverse order or in a different order. For these excitation wavelengths sets of dyes with yA=yB=yC=yD=yE=5 dye members, which can be separated spectrally or separated by combining spectral with fluorescence lifetime information can be easily constructed from commercially available fluorescent dyes. As illustrated in FIG. 7B the fluorescent dyes are preferably sequentially excited, such that all dyes have been excited and their fluorescence emission has been detected at least once in one round of reading out.

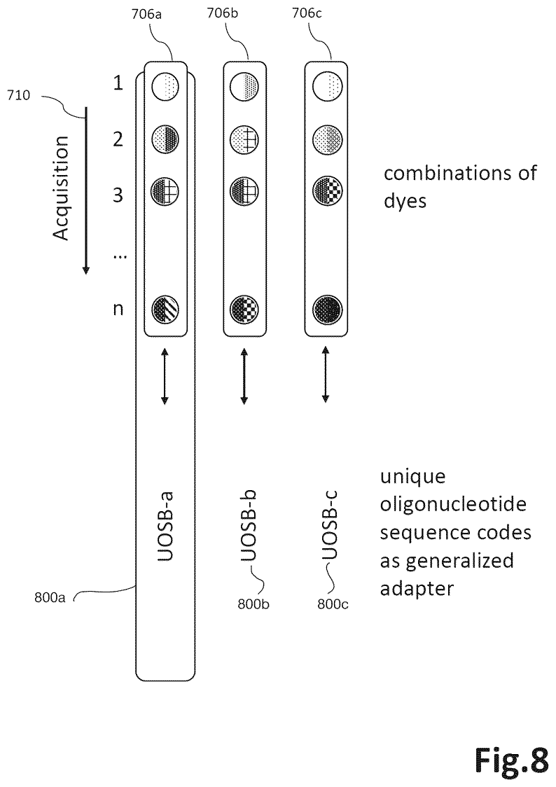

FIG. 8 shows a particularly preferred embodiment of the invention in which combination of dyes 706a, 706b, 706c, are mapped to unique oligonucleotide sequence barcodes (UOSBs) 800a, 800b, 800c in a 1:1 relationship. This is advantageous as it allows the linking of a combination of dyes 706a, 706b, 706c to a particular marker, which carries the corresponding complementary sequence barcode 800a, 800b, 800c, in a reversible fashion. This means that a library of combination of dyes can be constructed and connected to a library of oligonucleotides. To render an affinity reagent compatible with the process one would simply attach the complementary sequence barcode (UOSB) to the affinity reagent. This embodiment is well suited for an iterative process with changes to the encoding between multiple rounds as described below.

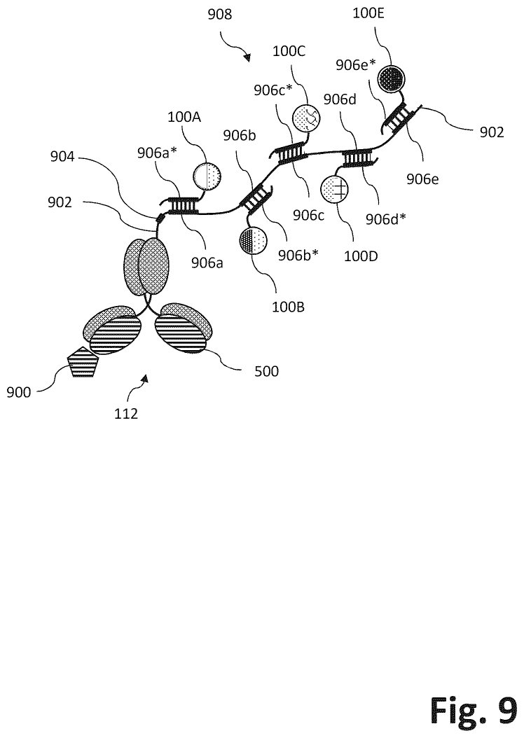

As shown in FIG. 9 the oligonucleotide sequence barcodes may also be used for the different dyes species in this case the sequences 906a*, 906b*, 906c* direct the conjugated dye to a complementary binding site 906a, 906b, 906c on the linker, which may well be a linker 902 comprising an oligonucleotide or longer sequence of DNA, RNA, LNA, morpholino or other artificial nucleic acid. Taken together the linker 902, which may contain multiple distinct elements, and the combination of dyes make up the reporter 908. FIG. 9 further shows a schematic example: a target molecule or analyte 900 is bound by an affinity reagent with corresponding specificity 500. This is schematically depicted by the pattern they share in the Figure. The affinity reagent is further connected directly or indirectly to a linker 902, which comprises a unique barcode of attachment sites 906a to 906e, which are complementary to the sequences 906a* to 906e* of the reporter, which include the fluorescent dyes or labels 100A to 100E. The linker further optionally may include a cleavage site 904.

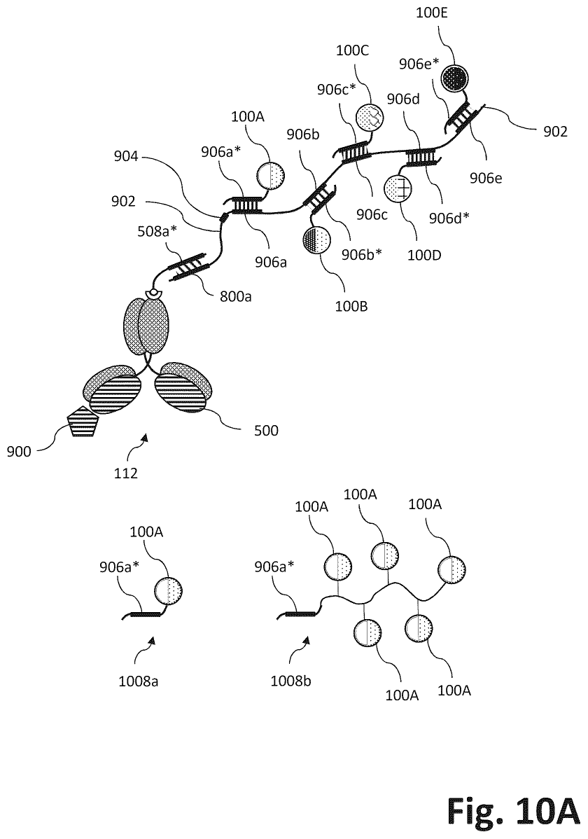

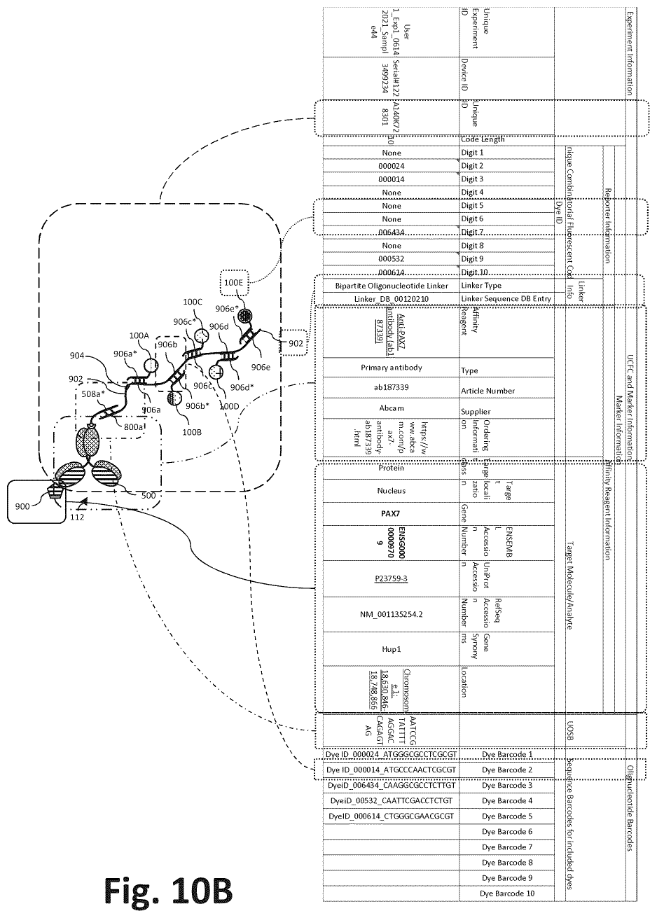

FIG. 10A illustrates that multiple dyes of the same species may be added to a tree-like structure, which might be an oligonucleotide a peptide or in particular a sugar. Sequence 906a* of a reporter may be attached to a single fluorescent dye or label 100A, as shown in FIG. 9 and on the left of FIG. 10A as 1008a. Alternatively, sequence 906a* of a reporter may be attached to a tree-like structure, comprising a plurality of fluorescent dyes or labels 100A, as shown on the right of FIG. 10A as 1008b.

FIG. 10B schematically illustrates how the combination of dyes and/or information relating to at least one of the combination of dyes, the affinity reagents, the markers, the targets molecules can be stored in a database and/or a memory device alongside a unique identifier (unique ID) and related information such as the dyes it contains, which are identified by a Dye_ID, as well as information about the assigned marker. Which may include information about the linker, the reporter, the marker and a UOSB (unique oligonucleotide sequence barcode) as well as further sequence barcodes, in case that an oligonucleotide linker 402 is used in which dyes are attached by using dyes-specific sequence barcodes. The information stored in a memory device may further contain information about the affinity reagent such as validation data, recommended dilutions, species of origin, data on cross reactivities, the target molecule, accessions to commonly used gene, transcript and protein databases. FIG. 10B further schematically illustrates where which type of information relating to a marker like 112 may be stored. Relational databases may be used to keep track of the relevant information in an efficient way, which allows the rapid retrieval and storage requiring a minimal amount of memory on a memory device.

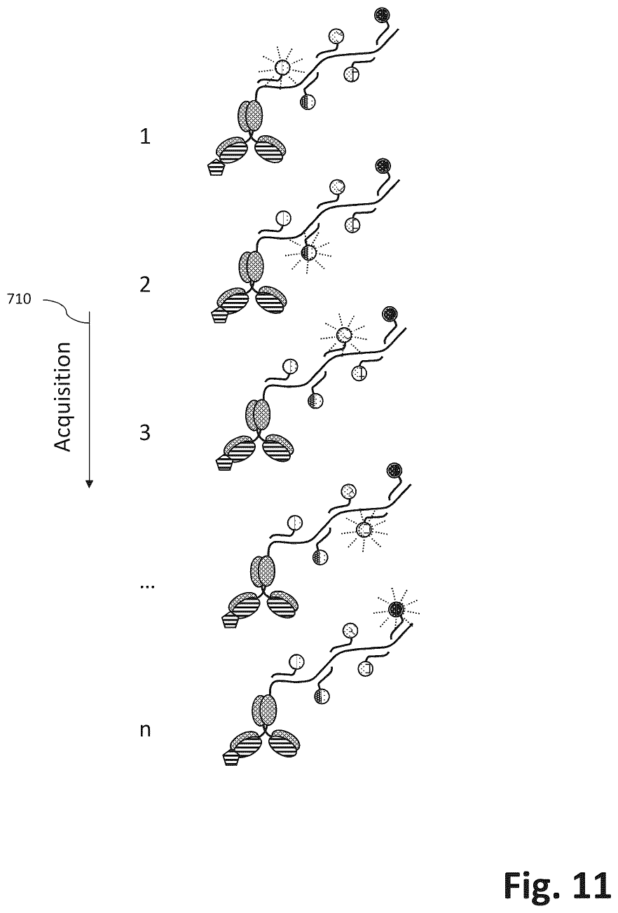

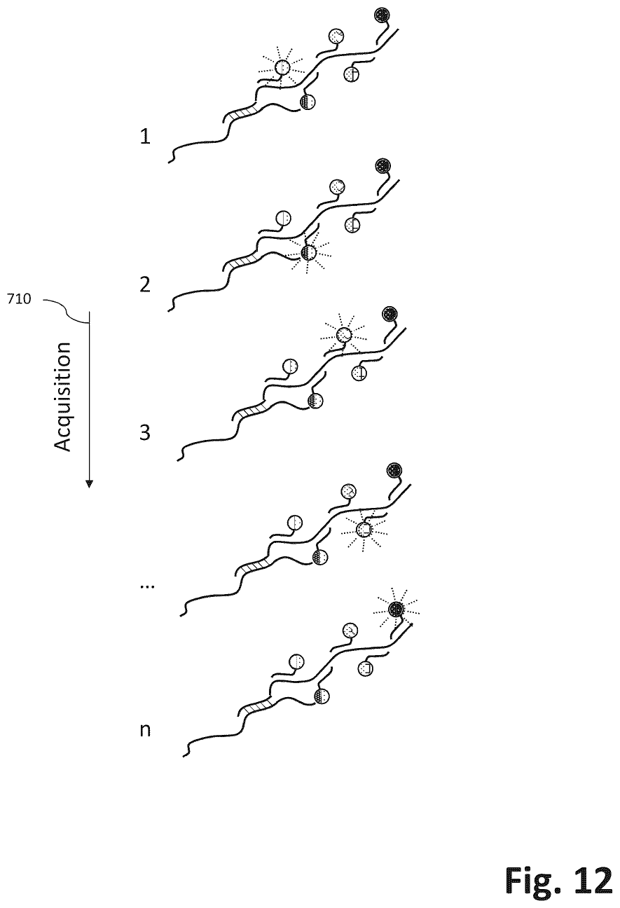

FIG. 11 illustrates the sequential readout of a marker bound to a target molecule, wherein the affinity reagent is an antibody and the target molecule a protein. The readout is accomplished by directing excitation light of different wavelengths towards the biological sample in which the markers are specifically attached to the respective analyte by a readout/acquisition sequence 710 indicated with 1 to n. FIG. 12 schematically shows the same process as in FIG. 11, but with a nucleic acid target for example a DNA or RNA target, which is bound by hybridization to a complementary oligonucleotide marker, which may comprise a DNA, RNA, PNA, LNA, morpolino, or other form of artificial nucleic acid.

FIG. 12. illustrates the sequential readout of a marker bound to a target molecule, wherein the affinity reagent is an oligonucleotide and the target molecule is DNA or RNA sequence.

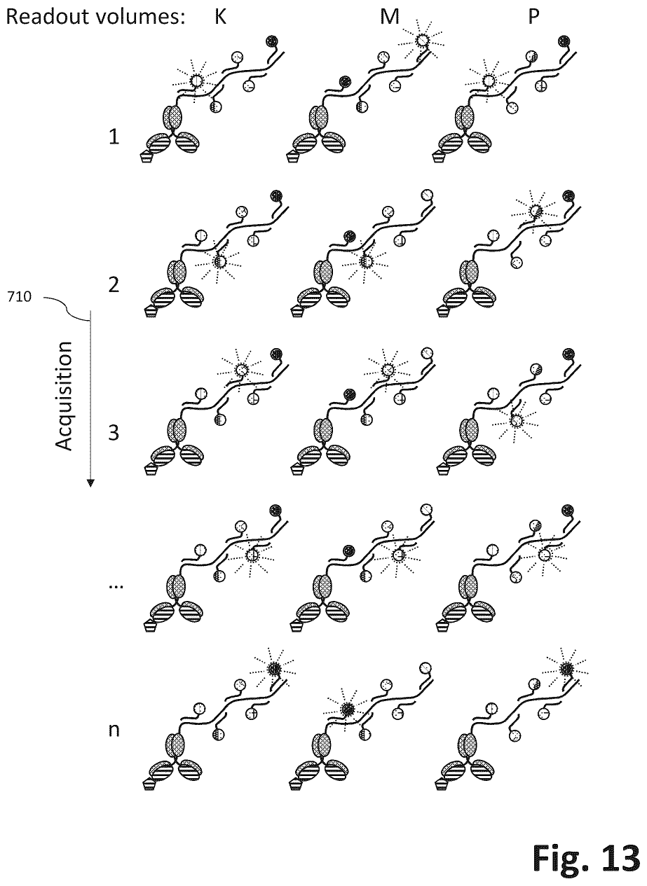

FIG. 13 schematically shows the same process as in FIG. 11 for three spots or readout volumes and illustrates the independence of the combinatorial code from the spatial positioning of reporter oligonucleotides on the linker oligonucleotide barcode. Readout volumes K, M, and P contain a single target molecule each of the same species bound by one marker molecule each of the same specificity, but with a linker in which the attachment sites to an identical combination of dyes are placed differently. As illustrated irrespective of the placing dyes the same readout sequence is obtained in this case for readout volumes K, M, P so as long as the spacing of dyes is substantially within the confines of the readout volume i.e. not resolved spatially by the optical system used for detection.

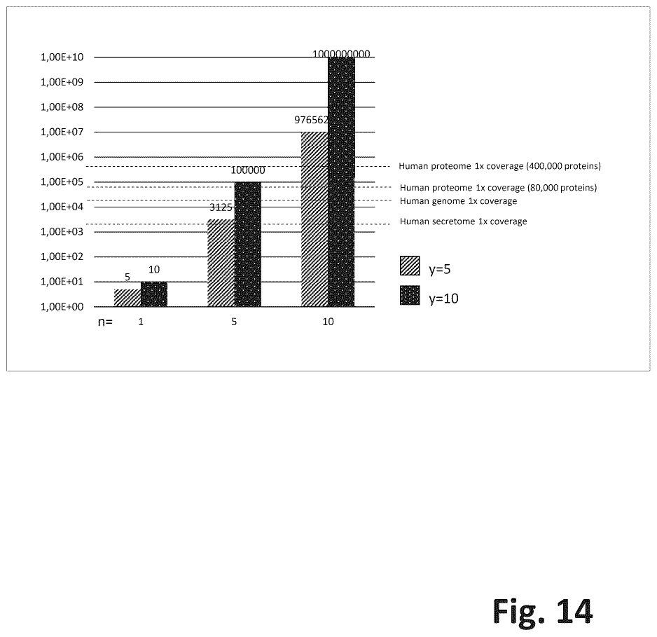

As illustrated by the bar chart in FIG. 14 and discussed above the exponential nature of the encoding is the basis for the statistical power of the approach. FIG. 14 shows another example, wherein set-based encoding is being used, wherein n=5 sets of dyes are excited at 405 nm (set A), 488 nm (set B), 565 nm (set C), 630 nm (set D), at 700 nm (set E) and each set contains 5 dyes, i.e. yA=yB=yC=yD=yE=5. In this case 3125 unique codes can easily be generated which is sufficient to provide uniquely labeled markers for each protein in the human secretome and thus enables secretome profiling in a single round, i.e. without necessitating an iterative process for staining, readout, dye deactivation. Further this means that the entire human genome and its main gene products can be uniquely labeled in an iterative process with 1-10 rounds of staining, reading out, deactivating the dyes. For comparison, modern multiplexing solutions offer around 60+ biomarkers to be imaged based on fluorescence imaging in 12 rounds of imaging. Similarly, modern cytokine profiling solutions allow the readout of around 40 cytokines (a special class of secreted molecules) in one round of imaging. For this reason, the method disclosed here is a significant improvement over the prior art and will allow the characterization of for example immune cells based on whole secretome analysis. This is particularly relevant in immunology, infection biology, and immunooncology to analyze the immune cells for their functionality and effectiveness and to derive predictive biomarkers that allow the stratification of patient populations. For example, it may be possible based on such analysis to predict whether a person infected with a certain virus, such as SARS-CoV-2 for example may have a mild or severe course based on the whole secretome profiling of immune cells. Similarly, it may be possible to predict whether a tumor patient has a good or poor prognosis without treatment, whether immune cells are competent and likely effective or already immunosenescent or ineffective for other reasons. Similarly, whole secretome profiling helps to predict the effectiveness of genetically modified immune cells such as CAR-T cells as well as to stratify patients into responders and non-responders for various forms immunotherapy (cell-based or non-cell-based). Further it may be possible, based on such analysis to predict which intervention for example combination of drugs may activated a weakly effective immune cell populations.

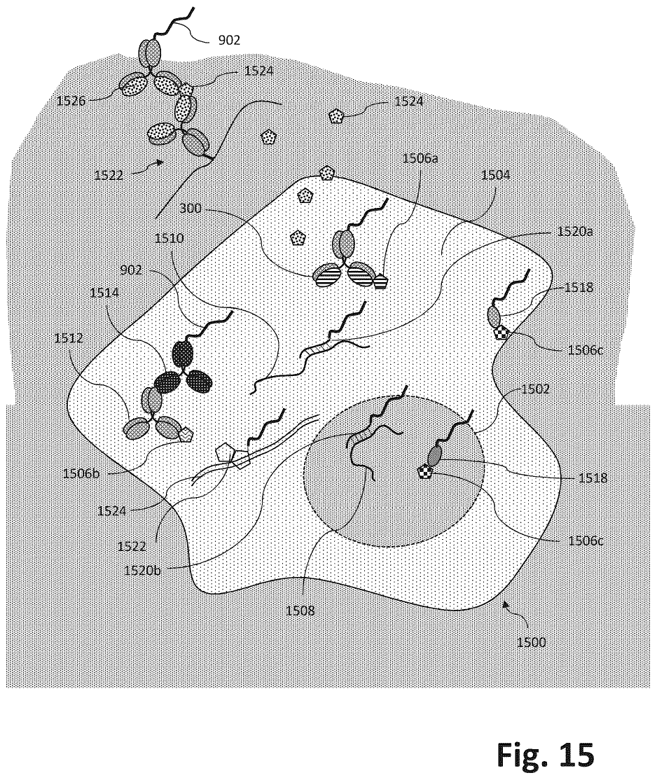

FIG. 15 is a schematic drawing of all cell 1500 as an example for a biological sample. In FIG. 15 markers are only shown with their linkers 902 and the dyes 100 are omitted (i.e. not shown) for the sake of clarity. The cell comprises a nucleus 1502 and cytoplasm 1504, each comprising structures known as epitopes 1506a-c to which antibodies attach themselves. This is exemplary shown for two markers 112 that comprise single-domain antibodies 1518 that bind to epitopes 1506c located in the nucleus 1502 and the cytoplasm 1504, respectively. Affinity reagents may be connected to a linker 902. Yet another marker comprises a primary antibody 1512 and a secondary antibody 1514 conjugated to a linker 902. The primary antibody 1512 is attached to an epitope 1506b in the cytoplasm 1504 and the secondary antibody 1514 is attached to the primary antibody 1512. Further, FIG. 15 shows two markers 1520a and 1520b comprising labeled oligonucleotide sequences as their affinity reagents. These two markers 1520a and 1520b are each attached to a complementary sequence 1508, 1510. A first of these two markers 1520a is attached to an RNA sequence 1510 in the cytoplasm 1504. A second of the two marker 1520b is attached to an DNA sequence 1508 in the nucleus 1502. A marker comprising a toxin 1522 (e.g. Phalloidin) as its affinity reagent is attached to an actin filament 1524.

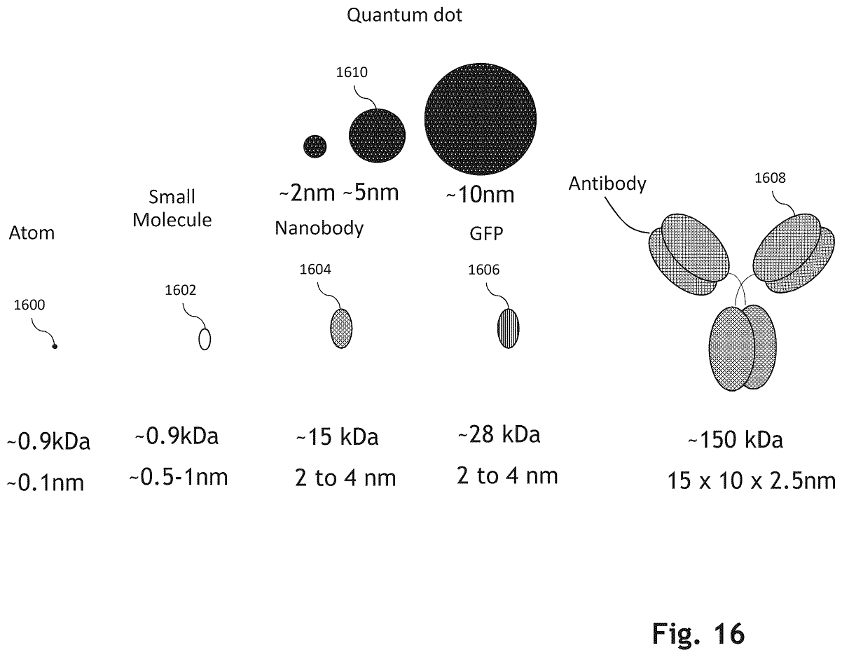

FIG. 16 schematically illustrates size ranges of relevant structures in the context of immunobased assays and immunohistochemistry. Shown are roughly to scale for the sake of illustration an atom 1600, a small molecule 1602, a single-domain antibody or nanobody 1604, a GFP molecule 1606, an antibody 1608, a quantum dot/polymer dot/graphene-based dot/other nanostructure in the range of 2-10 nm 1610.

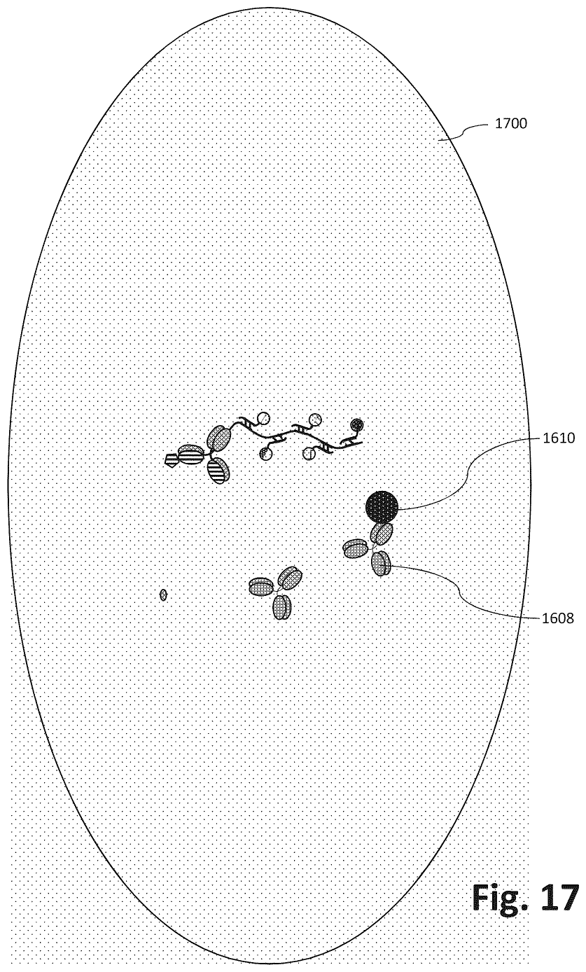

FIG. 17 schematically puts an antibody 1608 coupled to a quantum dot 1610 of 10 nm diameter in a PSF 1700 of a 1.4 NA objective. FIG. 11 and FIG. 12 illustrate that the optical resolution is not sufficient to readout the placing reporter oligonucleotides on the linker oligonucleotide barcode, which is why despite the difference in placing in the example shown in FIG. 13 the readout for spots K, M, P is exactly the same.

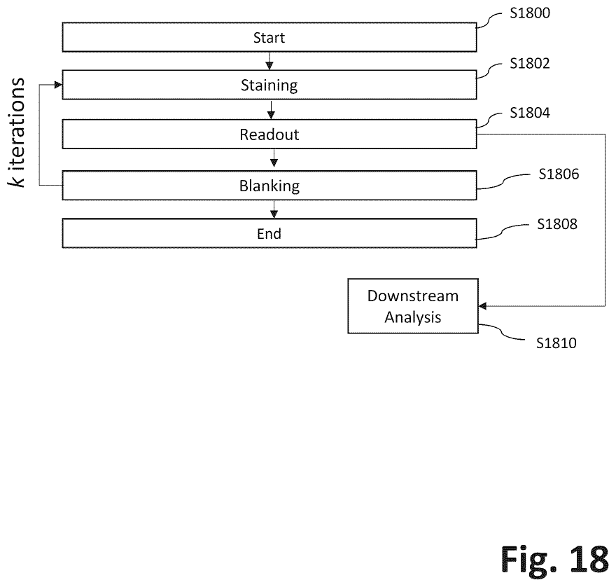

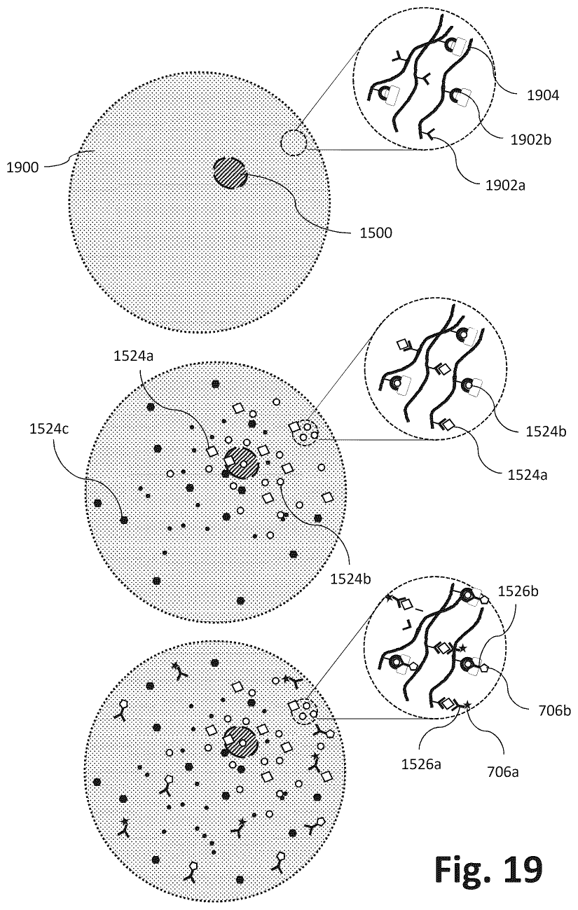

FIG. 18 schematically shows an iterative staining and imaging process, which is known from prior art and used in the context of multiplex imaging. The process starts in step S1800, in step S1802 the sample is stained. Markers are brought into the sample at this step and contact their predetermined structures and mark them. Commonly these markers are directly-dye conjugated in the process disclosed in this document. Alternatively, markers are generally indirectly dye conjugated and may be brought into the sample with reporters attached or without reporters. In the latter case the reporter may be brought into the sample, but dynamically associate and dissociate from the marker. Reporters may be brought into the sample in step S1802. In step S1804 the reporters are sequentially excited and read-out. In step S1806 the reporters are being inactivated by means of bleaching or removal from the sample and the process may be re-iterated or end in step S1808. Alternatively, the sample may be fed into a downstream analysis process step S1810 after the readout step S1804.

Optionally, following the capture of the images or the non-image-based readout, the fluorescent dyes 100 are deactivated in step S1806. Deactivation is done in order to prevent the fluorescent dyes 100 from emitting fluorescence light in the future. Methods for deactivating a fluorescent dye 100 include bleaching the fluorescent dye 100, either by chemically inactivating the fluorescent dye 100 or by photophysical bleaching; or removing the fluorescent dye 100 from the sample. In order to remove the fluorescent dye 100 from the sample, the connection between the primary affinity reagent 108 to 118 the predetermined structure or target molecule or analyte 900 has to be severed. This can be done for example by antibody elution in case the affinity reagent is an antibody 108 to 118. Alternatively, the fluorescent dye 100 could be removed from either the primary affinity reagent 108 to 118 or the secondary affinity reagent (not shown for sake of clarity). This can be done for example through enzymatic cleaving at a cleavage site 904 of the peptide 4802 or oligonucleotide 402 binding that connects the fluorescent dye 100 and the affinity reagent 108 to 118. It is also possible to reversibly bind the fluorescent dye 100 to the affinity reagent 108 to 118, e.g. through oligonucleotide hybridization and the use of barcoded antibodies. In the case of oligonucleotides 116, which are hybridization-based the oligonucleotides may be hybridized and dehybridized by using standard in situ hybridization or fluorescent in situ hybridization (FISH) protocols. In this case it is possible to hybridize the fluorescent dyes onto the linker and bringing the fully constituted marker into the sample. Alternatively, or in addition one could stain the sample with the affinity reagents 108 to 118 bearing a barcoded linker 902 and adding the dyes at a later point in time. Unlabeled primary affinity reagents 122 to 132 are likewise shown in FIG. 1.

FIG. 19 schematically shows a cell-based assay for secreted proteins. The illustration shows a region 1900 surrounding a cell 1500. This region may be virtual volume, in which detected secreted proteins are assigned to the included cell 1500 or it may be a physical volume, when the cell 1500 is for example encapsulated in a discrete entity like a hydrogel bead. In both cases the cell 1500 is embedded into a matrix, such as matrices used for 3D cell culture including but not limited to hydrogels, agarose, cellulose, alginates, Matrigel™, collagens, bioinks, and similar biocompatible materials. This matrix may be monophasic i.e. the zone used for cell cultivation and the zone used for capture and detection of secreted molecules may be the same or polyphasic, i.e. there may be distinct zones for cultivation and capture and detection, which may comprise different materials. In the latter case a polymer such as polyacrylamide, polyethylene glycol, polylactic acid, poly(vinyl alcohol), polyoxazoline, polystyrene may be used for the capture and detection zone. In either case as shown in FIG. 19 capture affinity reagents 1902a, 1902b may be covalently attached to the matrix. Upon secretion of secreted proteins 1524a, 1524b, 1524c are released into the matrix. In the example shown in FIG. 19 secreted proteins 1524a, 1524b are then immobilized by the capture affinity reagents 1902a, 1902b as shown in the enlarged insert. The immobilization allows the detection of secreted molecules even following harsh washing conditions by a suitable marker 1526a, 1526b which are identified by their assigned combination of dyes 706a and 706b respectively.

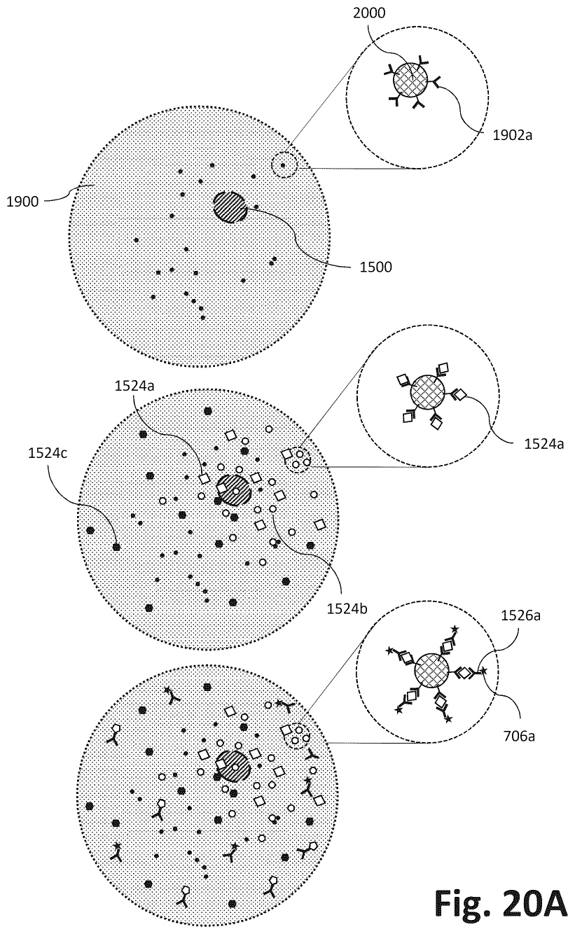

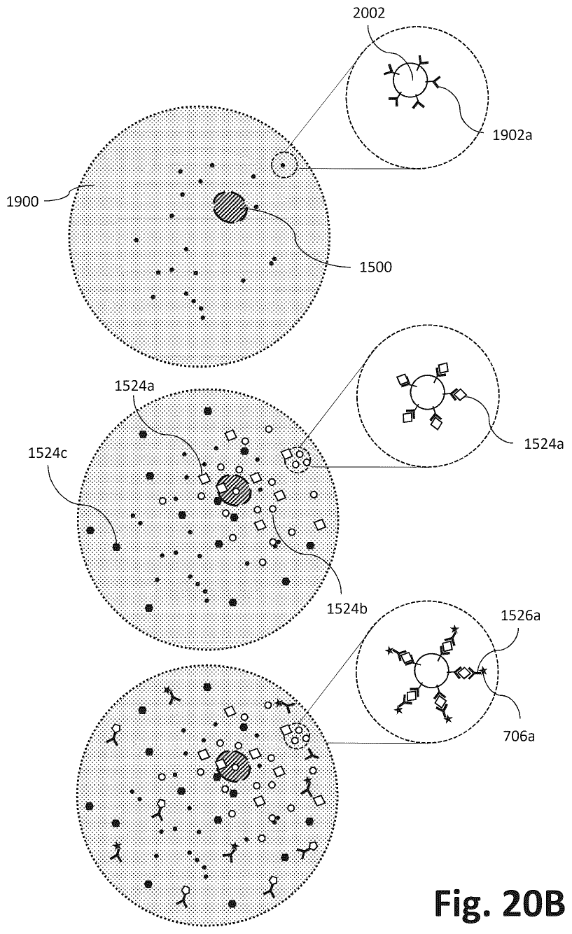

FIG. 20A schematically shows a particularly preferred embodiment of the invention in which the same process shown in FIG. 19 is using a different immobilization strategy. In this case capture affinity reagents 1902a, 1902b are immobilized on microbeads 2000, such as latex beads or polystyrene beads. Immobilization may be achieved directly by means of covalent coupling through a variety of chemistries (e.g. NHS, maleimide coupling) or through indirect coupling using oligonucleotide barcode linkers, biotion-(Strept)avidin interaction, or protein A, protein G mediated coupling. For example, a hydrogel bead of 50-250 μm diameter may be used to encapsulate a human cell 1500 (typical size 5-50 μm) and a mix of microbeads 2000 of 50-500 nm size carrying a plurality of capture affinity reagents 1902a, 1902b may be added during the embedding step. Due to the size of the microbeads they are immobilized in the hydrogel, which means that they are in a substantially stable position relative to each other and the circumference of the hydrogel bead. For this reason, they are ideally suited to define detection spots or detection regions as their size can be adjusted and chosen in relation to the numerical aperture of the readout device. The high local concentration of capture affinity reagents on microbeads is particularly preferable as this yields to a higher intensity per area, a better signal-to-background and renders a segmentation and analysis easy. Moreover, the high local concentration of capture affinity reagents on the microbeads leads to a pronounced avidity effect, which renders even weak and transient interactions accessible to this method, i.e. it renders the assay significantly more sensitive. Further microbeads with capture affinity reagents can be prepared in libraries and flexibly deployed for a variety of assays and different assay formats, such as assays in microtiter plates or in flow-through.

FIG. 20B schematically shows a particularly preferred embodiment of the invention in which the same process shown in FIG. 19 and FIG. 20A, wherein the capture affinity reagents are immobilized on a nanostructure 2002, such as graphene, carbontube, nanoruler or other DNA-origami-based nanostructure.

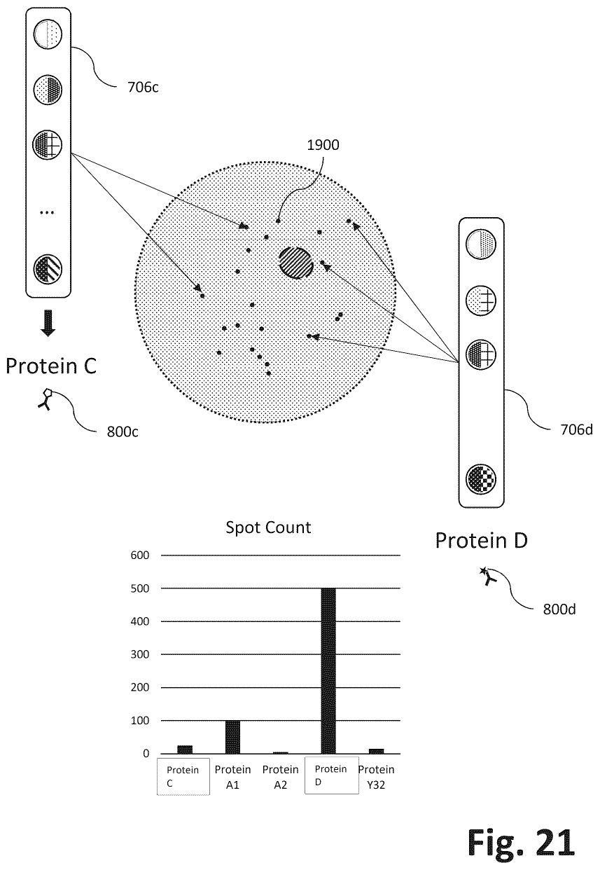

FIG. 21 schematically shows how microbeads and similar sized nanostructures in the range of 1-5 nm, 5-10 nm, 50-100 nm, 100-200 nm, 250-350 nm and 350 nm-1000 nm, and 1000 nm-5000 nm are particularly preferable for a variety of assays involving secreted proteins, which are depicted in FIGS. 19, 20A, 20B, as a random placing of the microbeads facilitates the disparate placing of detection spots and thus aids in readout. FIG. 21 schematically shows 2 spots identified by their unique combinatorial code 800c as Protein C and a respectively attached combination of dyes 706c and 3 spots identified by their unique combinatorial code 800d as Protein D and a respectively attached combination of dyes 706d. Further FIG. 21 illustrates how spots are decoded and counted. The bar chart on the bottom of FIG. 21 shows a schematic of a spot count result for several proteins. Alternatively, or in addition DNA-origami based nanostructures lend themselves well for this kind of assay, as they can be generated in relevant sizes and can be easily modified to contain attachment sites for fluorescently labeled oligonucleotides or other labels and functionalizations.

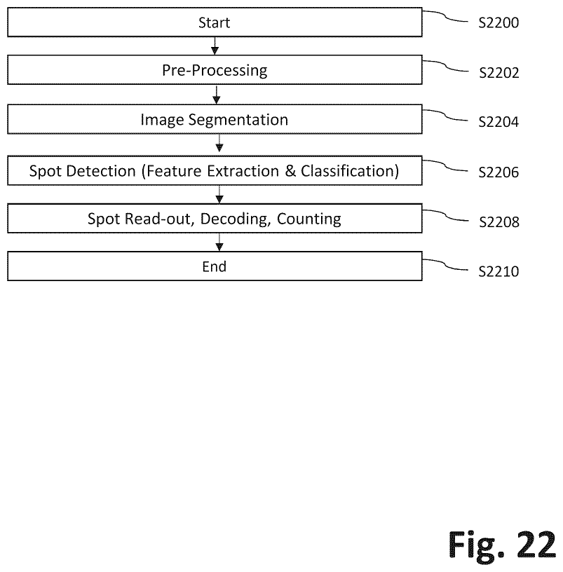

FIG. 22 shows a flowchart of a workflow for finding and reading out mono-species readout volumes in assays shown in FIGS. 19 to 21, that starts in step S2200. This may also be referred to as an image processing pipeline. Such an image processing pipeline may include at least one of the following: background removal, compression, filtering, denoising, enhancement, reconstruction, correction, deconvolution, multi-view deconvolution, multi-view registration, multi-view fusion, as well as other tools well known to someone skilled in the art of digital image processing and typically include an image segmentation step, which generates a set of segmented objects that may also be named features, as well as a feature classification step. For many of the afore mentioned image processing tools classical software algorithms and (modern) machine/deep learning or AI-based algorithms exist. In particular for pixel classification, image denoising, deconvolution, enhancement, segmentation, and classification of segmented objects/features machine/deep learning or AI-based algorithms exist and are known to provide superior performance to most classical algorithms. It might be advantageous to perform image analysis, image improvement and/or image deconvolution of the images generated by an optical read-out.

Certain steps may be omitted or repeated, other steps not shown in the FIG. 22 may be included in the image processing pipeline. Sub steps of the pipeline may be executed using classical image processing algorithms and/or machine learning, deep learning algorithms. The output of feature extraction might be a set of features, that includes features from the biological sample of a hydrogel bead in which the biological sample might be embedded such as its outer limitations, its volume or centre of mass, the subset of features derived from the structure outside of the sample including the mono-species readout volumes, as well as the subset of features derived from the biological sample.

In a step S2202 the image data is pre-processed, which may include background removal, by means of a variety of feature detection methods including segmentation and filtering. The constituent parts of the marker may be identified by their keypoint features, edges, and interest points/feature points. Feature or interest points may be any detectable object, for example a microbead 2000. A keypoint feature could be all the objects that have a certain neighbourhood for instance. For example, microbeads 2000 may be segmented and their centre of mass determined.

The image segmentation analysis in step S2204 can be carried out with at least one of: classical approaches, artificial intelligence based techniques including machine learning and neural-networks/deep learning, or other techniques including thresholding techniques, clustering methods, compression-based methods, histogram-based methods, edge detection, dual clustering method, region-growing methods, partial differentiation equation-based methods, variational methods, graph partitioning methods (for example Markov random fields), a watershed transformation, model-based segmentation, multi-scale segmentation, semi-automatic segmentation, trainable segmentation using various machine learning, neural network and artificial intelligence approaches for example pulse-coupled neural networks (PCNNs), and convolutional neural network (U-Net), recurring neural networks (RNNs) as well as object co-segmentation methods such as Markov networks, convolutional neural networks, or long short-term memory (LSTM), for example. Alternatively, or in addition, characteristics such as size and/or colour and/or fluorescent intensity and/or fluorescent lifetime can be used to identify the constituent parts of the marker from the image data. Various algorithms can be used for identification including Harris Corner, scale invariant feature transform (SIFT), speeded up robust feature (SURF), features from accelerated segment test (FAST), and oriented FAST and rotated BRIEF (ORB) are known and can be used to identify the constituent parts and/or features of the marker from the image data.

It is particularly preferable, that spot detection and/or feature extraction and/or feature classification analysis S2206 are performed leveraging machine and deep learning approaches, such as for example content aware feature enhancement. This is especially advantageous when the marker is generated using fluorescent microbeads, fluorescent nanorulers or similar structures as this allows neural networks to be pre-trained to perform content aware feature enhancement specifically for these features. Likewise, in this case substantial pre-training can be easily performed against a ground truth, i.e. image data from the hydrogel beads containing only the marker. Importantly, the ground truth can be generated on a different imaging system. In particular the ground truth can be generated on an imaging system that has a high optical performance with respect to e.g. numerical aperture, resolution, light-collecting efficiency, Etendue (flux), signal-to-noise, chromatic or spherical aberrations as well as any other imaging aberration. Importantly, the method can be implemented in a way that each run generates respective training data that potentially improves the quality of the generated image data by improving for example denoising, background removal, image correction, deconvolution, amongst others as well as the performance of feature extraction. Similarly, networks can be pre-trained using suitable reference samples to classify features. In particular networks can be pre-trained to classify a feature as the hydrogel bead, a fluorescent microbead, a fluorescent nanoruler, or similar, and a cell, a group of cells, or a different kind of biological sample.

Following to feature extraction and classification spots are readout, their combination of dyes is decoded in step S2208, by looking up the identity of the corresponding marker and its target molecule/predetermined structure in the memory device. Each spot labelled with a certain combination of dyes is then counted and the result is stored alongside the intensity information in a memory device. The process ends in step S2210.

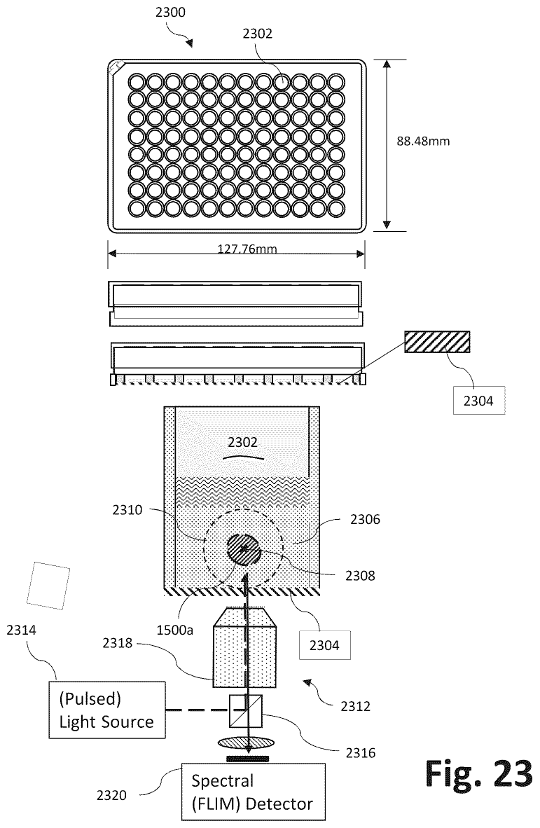

FIG. 23 schematically illustrates how the assays described in FIG. 19 to 21 can be performed in a standard microscopic sample carrier such as a microplate 2300 with 96 wells or sample containers 2302, each having a transparent window or bottom 2304. In this case the sample such as a cell 1500a is segmented and its centre of mass 2308 is found, a virtual readout volume 2310 assigned to cell 1500a is related to this reference point. The sample and the virtual readout volume 2310 is imaged or readout using a microscope or plate reader 2312 that has a light source 2314, a beam path with beam routing and beam splitting elements 2316, an illumination/detection optic 2318 (which may be through the same optic or different optics) as well as a detector 2320 configured to readout fluorescence intensity. The detector 2320 may also be a spectrometer as depicted in FIG. 2.

In a particularly preferred embodiment of the invention, the detector and the light source are configured to perform spectral fluorescence lifetime imaging which may be used for example with spectral FLIM phasors and provide a high dye separation capacity of the readout device.

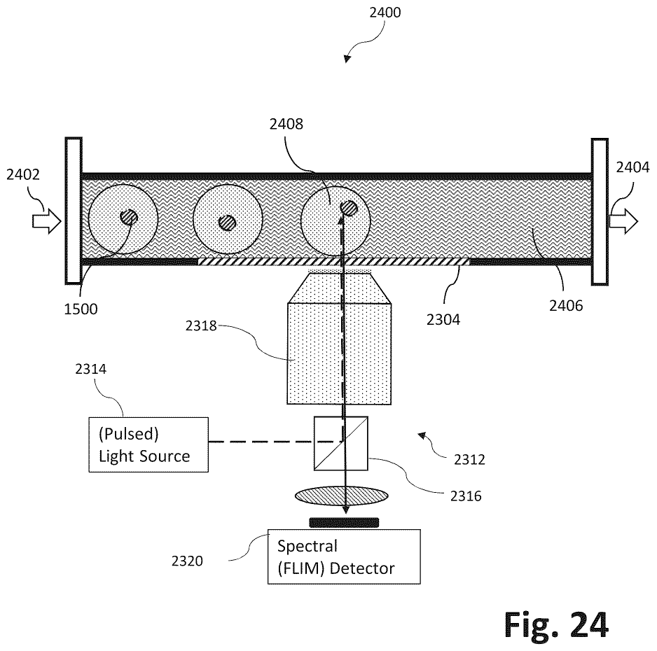

FIG. 24 schematically illustrates how the assays described in FIG. 19 to 21 can be performed in flow through in an imaging cytometer, a cytometer or a microscope configured to perform flow-through based imaging. The imaging system 2312 described in FIG. 23 is used in this case in conjunction with a flow cell 2400, with an inflow 2402, and an outflow 2404, at least one fluidic channel in which the immersion or flow medium 2406 flows. The samples 1500 in this case are embedded in hydrogel beads 2408, which is particularly advantageous as it protects the enclosed samples 1500 and enables the assays described in FIG. 19-21 to be performed in flow-through with very high throughput. Embedding and identifying biological samples in hydrogel beads is e.g. described in the PCT/EP2021/061754, the content of which is completely included herein by reference.

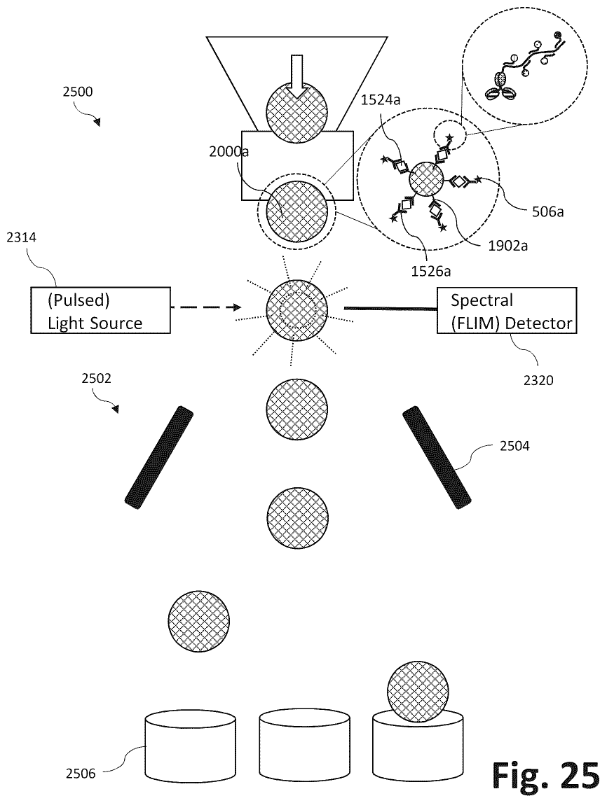

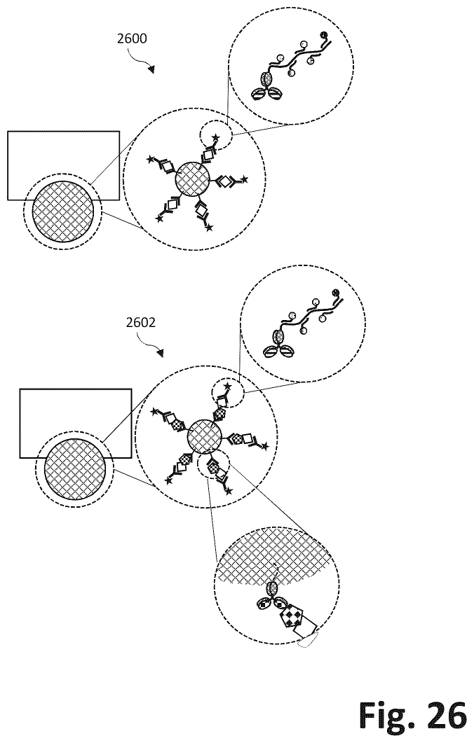

FIG. 25 shows a bead-based assay on a cytometer 2500, wherein microbeads 2000a that carry capture reagents 1902a, are excited by light from a light source 2314 and the emitted fluorescence is detected by a detector 2320. As shown in FIG. 25 each microbead 2000a may carry an affinity reagent of a given specificity 1902a rendering it a mono-species readout volume effectively. Such a capture bead 2000a may be incubated with a cell for a given amount of time in for example a well (compare FIG. 23) and then subject to cytometric analysis. In a particularly preferred embodiment of the method, this is used to perform whole secretome profiling using the described bead-based format and cytometry as a readout.

Alternatively, or in addition, the capture beads 2000a may be incubated with a lysate of a cell or the lysate of a sample or an environmental sample to detect the presence of an analyte in the sample. In a particularly preferred embodiment of the method, this is used to detect the presence of a large number of analytes in the same experiment using the described bead-based format and cytometry as a readout.

Optionally, the capture beads 2000a may be sorted using a fluorescence activated cell sorter (FACS) 2502, which uses deflector plates 2504 that direct beads into respective collection tubes 2506.

FIG. 26 shows that the assay described in FIG. 25 can be used to detect the presence of an analyte in the capture reagent-target molecule-marker-configuration 2600 shown on top, which might be configurations as shown in FIG. 19, 20A or 20B as well. Alternatively, or in addition the format maybe adjusted to the capture reagent-target molecule-interacting molecule-marker-configuration 2602 shown below. While the former allows for the presence of analytes to be analysed, the latter enables probing interactions of a certain analyte or a plurality of analytes, which are captured by the capture reagents, with interacting molecules (i.e. other analytes such as small molecules, proteins, nucleic acids), which are in turn readout by markers that mark the bound interacting molecules. Using the method disclosed in this document with such bead-based assays combines the advantages of bead-based assays such as a pronounced avidity effect that results from concentrating capture reagents on the beads with the advantages of the method disclosed in this document, which provides a way to detect a very high number of analytes and/or to assess a very high number of interacting molecules. The interacting molecules may be in solution or may be cell surface molecules expressed on cells. In this way bead-based assays using the capture reagent-target molecule-interacting molecule-marker-configuration 2602 may be used to detect the presence of cells based on a combination of multiple cell surface molecules. In this way the method may be used to detect the presence of certain cell types, which may be rare cell types such as circulating tumor cells or immune cells reactive to a certain antigen. Both of these assays are well suited in situations where a high number of events, a high number of analytes, high throughput or short time to result are desirable.

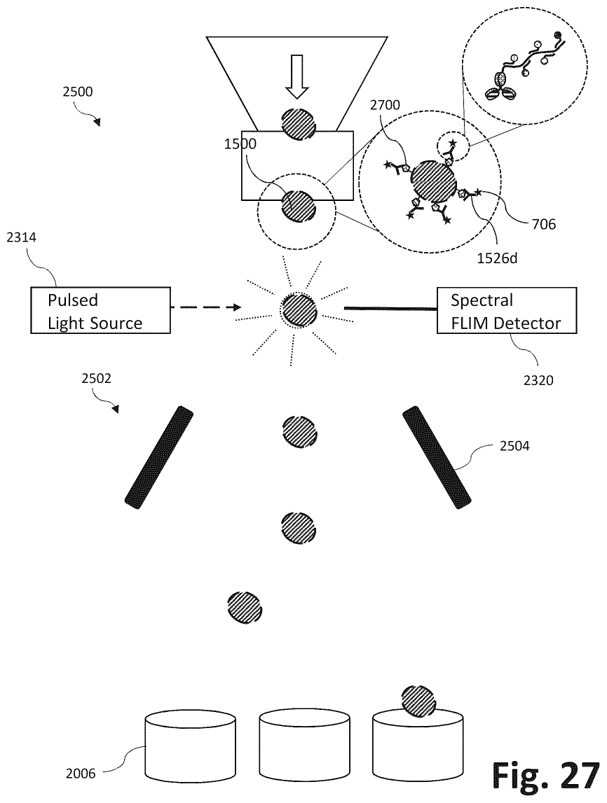

FIG. 27 shows a cytometer 2500 and a FACS 2502 similar to FIG. 25. In this case the samples in the stream of samples are single cells 1500. The assay schematically depicted in FIG. 27, illustrates a particularly preferred embodiment of the invention, wherein a very high number of markers is used to label proteins or other target molecules (e.g. sugars) that are bound to the cell-surface of cell 1500. This may be in particular cell surface receptors and proteins of the “cluster of differentiation” (CD proteins). These cell surface-bound target molecules 2700 are bound by respective markers consisting of an affinity reagent 1526d and a combination of dyes 706.

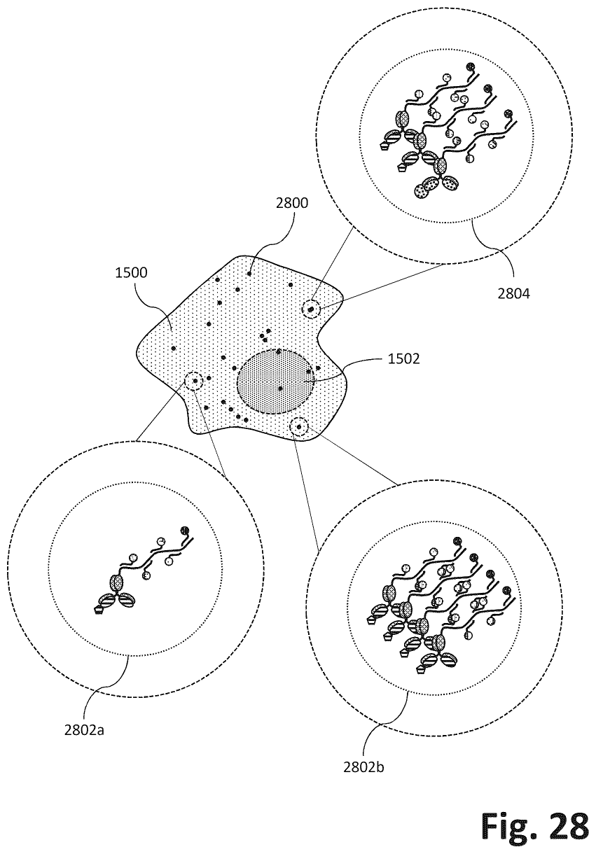

In a particularly preferred embodiment of the invention the assays described in FIGS. 19-27 are configured such that they generate primarily mono-species readout volumes, see e.g. 2802a, 2802b in FIG. 28, which can be directly readout, i.e. wherein the readout directly decodes the identity of the marker.

In a further particularly preferred embodiment of invention the assays described in FIGS. 25-27, are used to perform ultra-high plex cytometry, such as cytometry with 100-1000, 1000-2000, 2000-10,000, 10,000-30,000, 30,000-100,000 and above 100,000 markers which are readout in the same experiment and preferably in a single round (multi-species readout volume), see e.g. 2804 in FIG. 28.

In a further particularly preferred embodiment of invention the assays described in FIGS. 23 and 24, are used to perform ultra-high plex imaging in a standard sample carrier or in flow cell in flow-through, such as imaging with 100-1000, 1000-2000, 2000-10,000, 10,000-30,000, 30,000-100,000 and above 100,000 markers which are readout in the same experiment and preferably in a single round or alternatively in multiple rounds of staining, imaging, dye deactivation (multi-species readout volume).

FIG. 28 illustrates the difference between a mono-species readout volume 2802a and 2802b and a multi-species readout volume 2804. A mono-species readout volume as shown in FIG. 28 contains markers of the same reactivity or specificity 500 either in a single copy 2802a or in multiple copies 2802b. For this reason, the readout sequence from a mono-species readout volume 2802a or 2802b is identical to one combination of dye from the set of marker-assigned combinations of dyes. This means that in order decode a mono-species readout volume, i.e. identify the included marker species it is sufficient to simply readout the mono-species readout volume and retrieve the corresponding marker ID by comparing the readout sequence to the combinations of dyes from the set of marker-assigned combinations of dyes stored in the memory device. FIG. 28 further illustrates that a multi-species readout volume 2804 contains at least two markers of distinct reactivity or specificity 500.

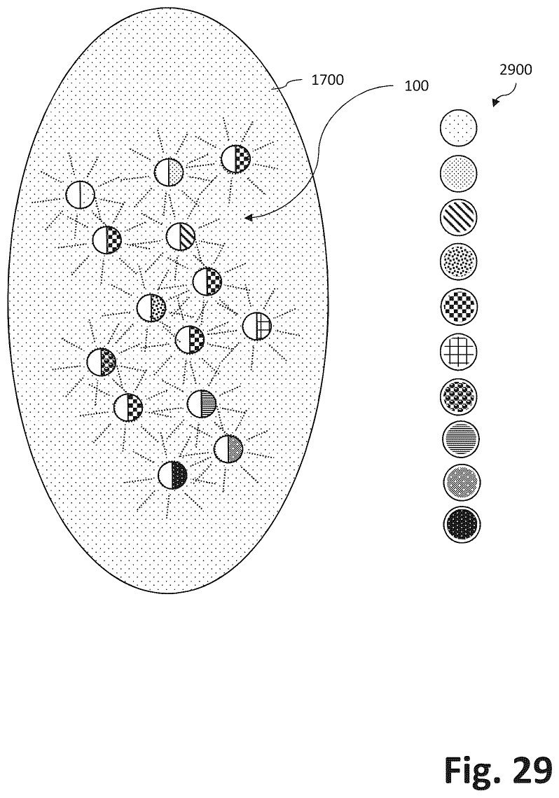

In contrast to a mono-species readout volume a multi-species readout volume cannot simply be decoded by obtaining the readout sequence and retrieving the underlying markers. As illustrated in FIG. 29 a multi-species readout volume may contain a number of different markers. In FIG. 29 a confocal readout volume or effective point-spread function 1700 and a number of dyes 100 of for example the first set of dyes are shown. For the sake of clarity of the illustration each dye corresponds to one marker molecule of which the remainder is omitted (i.e. the affinity reagent and the other dyes of the respective combination of dyes). If for example such a plurality of different markers is excited with the first excitation light the first dyes in the respective combination of dyes will be excited and emit fluorescence light. This light will be detected and will be separated into the respective dye channels, this is schematically depicted by the column of circles which visually represent a readout sequence 2900 on the right. The circles on the right only have a single pattern, which reflects all the properties used for dye separation, whereas the circles in the PSF 1700 correspond to dyes 100 and have a left half pattern corresponding to the excitation spectrum and a right half pattern corresponding to the properties of the dye that can be used for dye separation (e.g. emission spectrum, fluorescence lifetime, excitation fingerprint). In the example shown in FIG. 29, after the first excitation light has been applied to the sample the emitted fluorescence light has been detected, the method has registered the presence of 10 dyes represented by ten circles with ten distinct patterns. Further the method has registered the intensities of these dyes. In analogous fashion now all other excitation lights are applied and the fluorescence light is detected as described above. In this way a first readout sequence (e.g. from the first iteration of an iterative staining-imaging-readout process) is obtained. Depending on the number of distinct markers in the spot this may lead to a significant number of codes that can be subsumed under this readout sequence and likewise significant uncertainty as many of the subsumable codes might be false-positives, i.e. their assigned markers may not be physically present in the confocal volume or effective point spread functions.

The following discussion first discusses strategies to mitigate this problem and then turns to a discussion about a robust solution of this problem second.

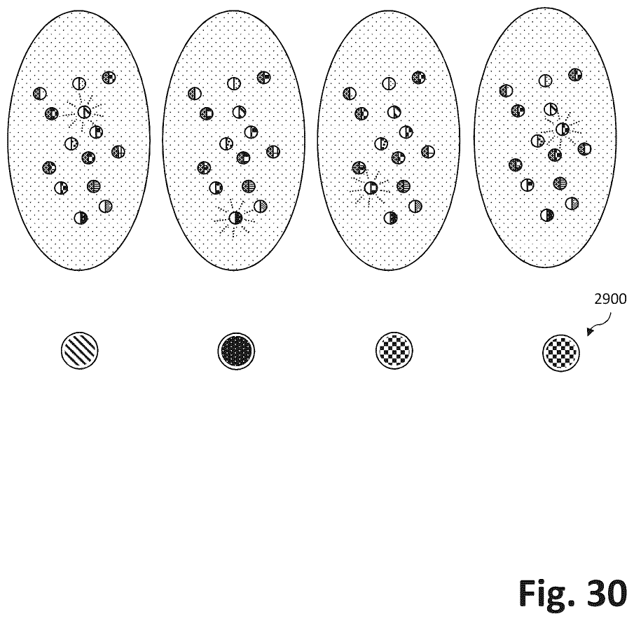

In terms of mitigating the challenge to decode multi-species readout volumes, it is possible to use blinking of dyes and methods that are used for localization microscopy, which effectively turns a multi-species readout volume into a number of temporally separated sub-diffraction localized mono-species readout volumes. This is depicted by only one dye lighting up in each of the PSFs shown in FIG. 30.

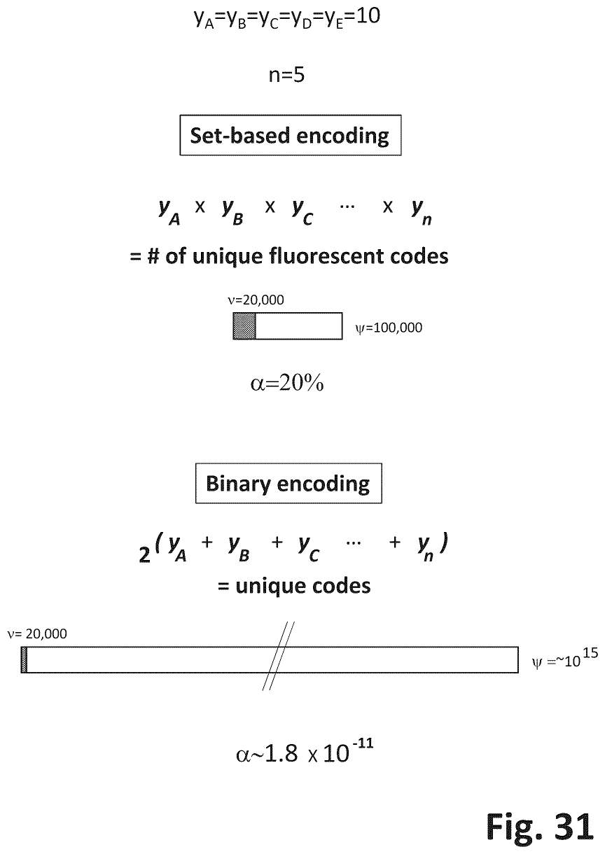

In a particularly preferred embodiment of the invention, the challenge of multi-species readout volume decoding is solved in principle. As illustrated in FIG. 31 for an example in which yA=yB=yC=yD=yE=10 and n=5 set-based encoding generates □=100,000 and binary encoding generates □□~1015 combinations. This means that □ is 20% and ~1.8× 10-11. In other words, and as illustrated by the bar charts only a small or extremely small fraction of the available codes is actually assigned to a marker (grey rectangle).

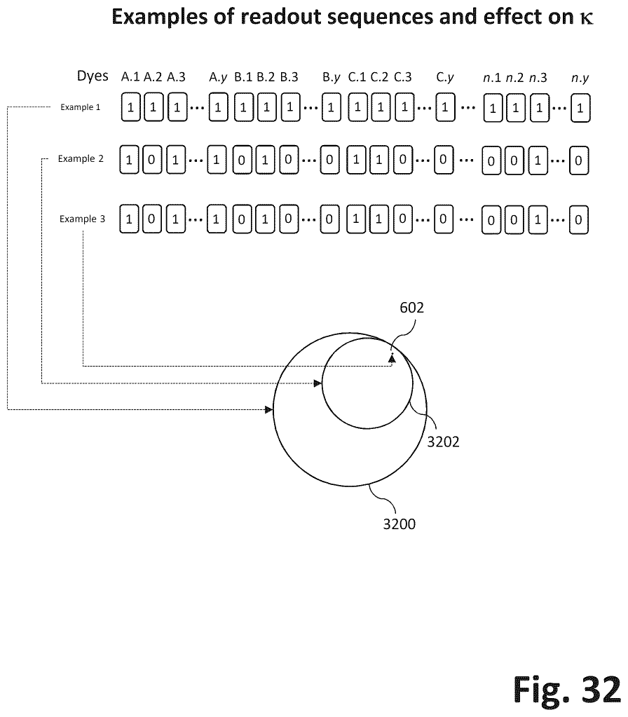

FIG. 32 illustrates how different first readout sequences impact □ the number of combinations of dyes subsumable under a given first readout sequence. In an “Example 1” a readout sequence is observed, in which each digit corresponding to a dye A1. . . . , n.y, shows a “1”, i.e. all dyes were observed. In this case the entire plurality of combinations of dyes S1, 3200 can by necessity be subsumed under the readout sequence of “Example 1”. This is schematically depicted by the big circle 3200. In an “Example 3” the readout sequence allows the unambiguous identification of a single combination of dyes 602 and thus a single marker (mono-species readout volume). In an “Example 2” a readout sequence is obtained, under which a significant number of combinations of dyes can be subsumed forming the “set of combinations of dyes subsumable under the first readout sequence” 3202. This latter case is the realistic case for multi-species readout volumes and is further discussed in the following.

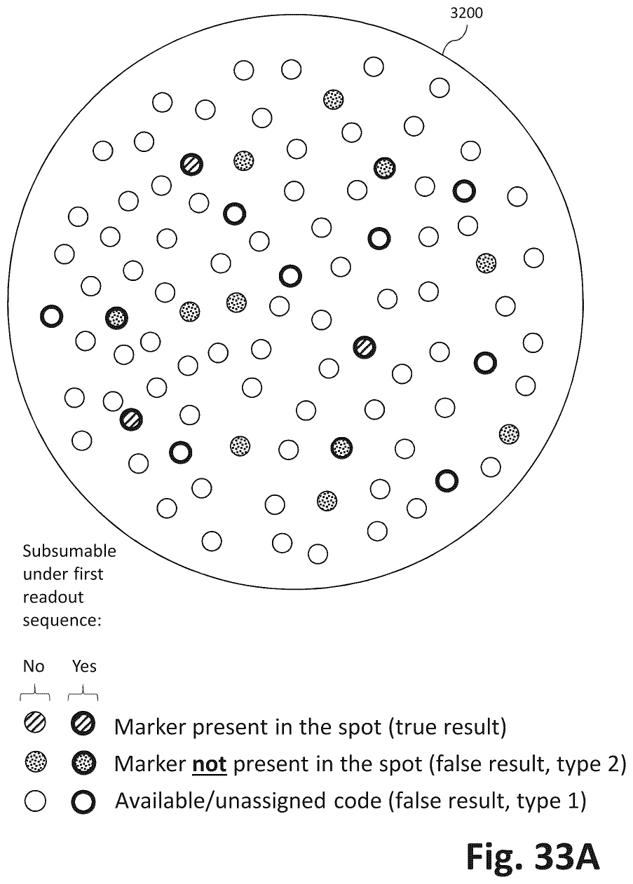

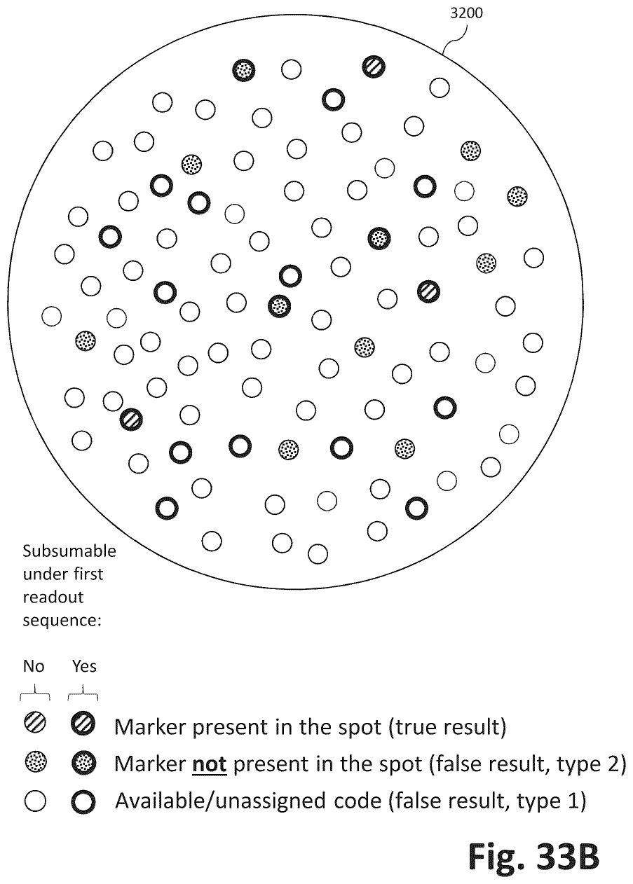

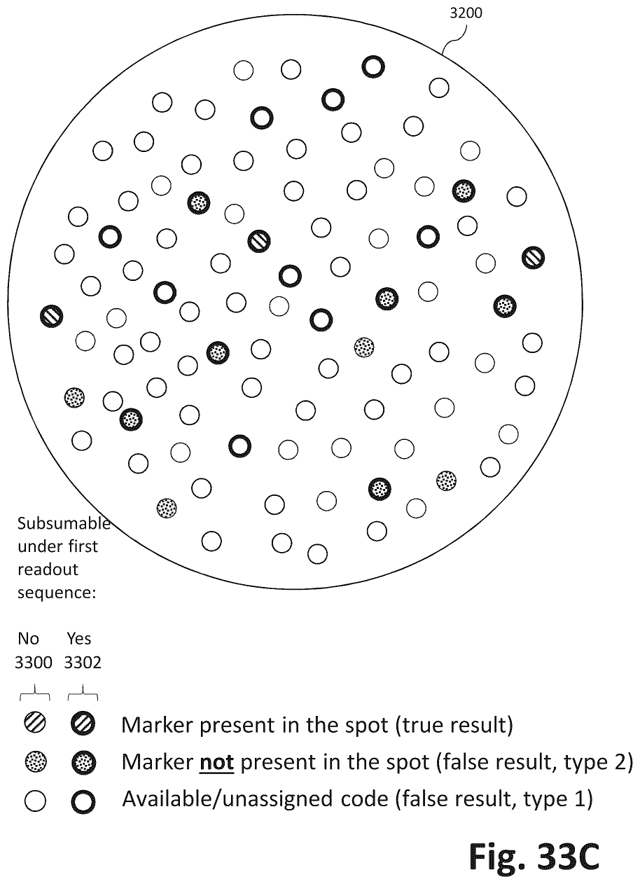

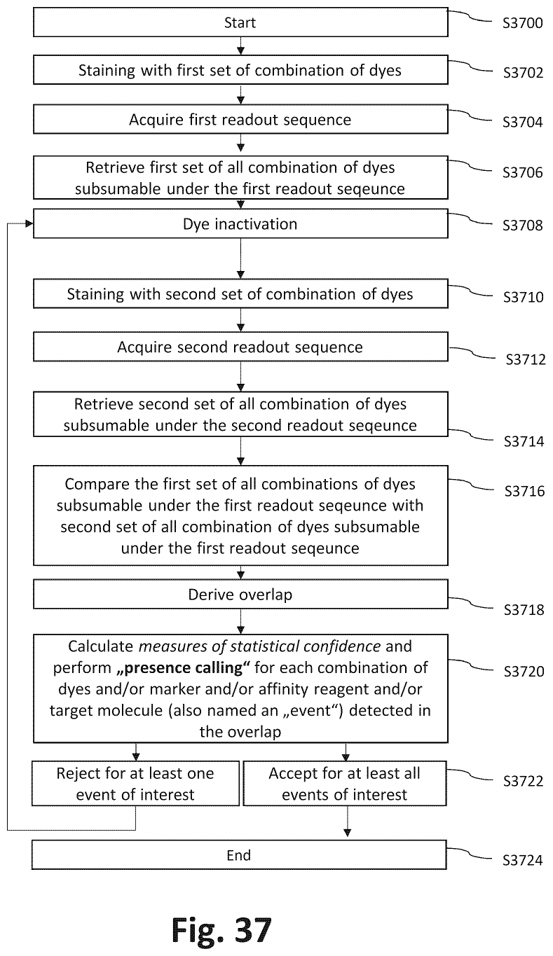

FIG. 33A illustrates this schematically, in this example a first readout sequence was obtained and the entire plurality of combinations of dyes S1, 3200 is shown. In the illustration each circle corresponds a combination of dyes 602. Most circles are filled white and correspond to combination of dyes which are not assigned to a marker. Some circles are filled with a pattern either (large confetti pattern or diagonal stripes) and correspond to combination of dyes assigned to markers. Most of the circles are drawn with a thin contour line and resemble all combination of dyes that cannot be subsumed under a first readout sequence. Only some circles are drawn with a solid black thicker contour line and represent the combination of dyes subsumable under a first readout sequence. In analogy to FIG. 33A, FIGS. 33B and 33C illustrate the scenario under a second and a third readout sequence following to reassignment of combination of dyes to affinity reagents or vice versa and therewith reassignment of combination of dyes to markers or vice versa. In this sense FIG. 33A illustrates the first iteration in an experiment, FIG. 33B the second, and FIG. 33C, wherein iteration refers to an iterative staining, imaging, dye inactivation process shown in FIGS. 18 and 37. In between the steps the plurality of sets of combinations of dyes S1, 3200 may stay the same as shown in the illustration, but their assignment is changed randomly. Alternatively, or in addition the assignment may be changed deterministically. Alternatively or in addition, a second plurality of sets of combinations of dyes S1.2 based on a second suitable “rules of forming combinations of dyes” may be formed and used in the second iteration (not shown in the illustration for the sake of clarity). This is best observed by swiftly changing from FIG. 33A-C, which animates this example. This can be compared to repeated drawing with putting back/replacement.

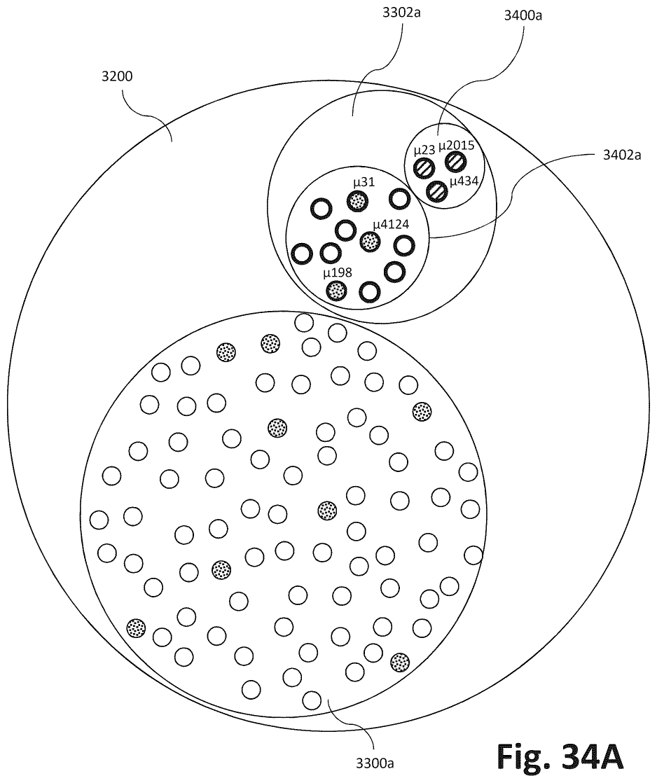

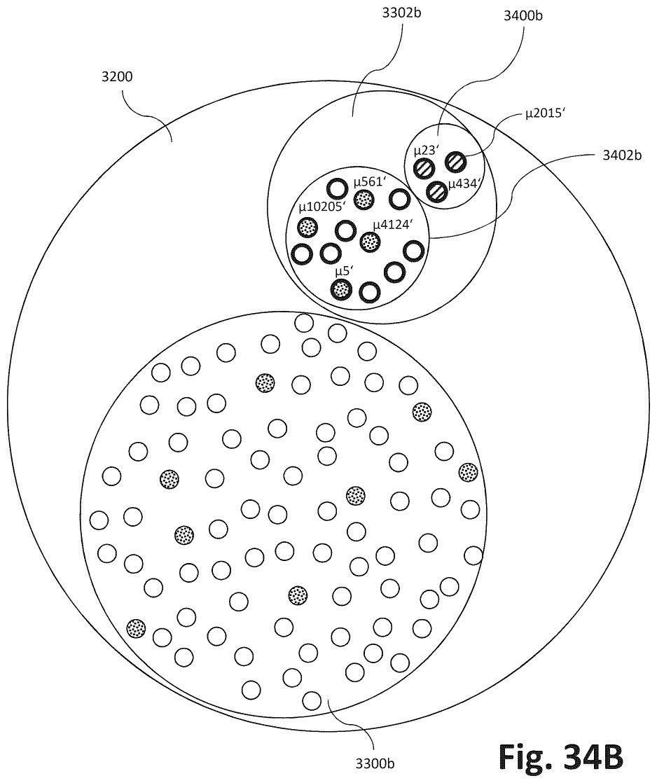

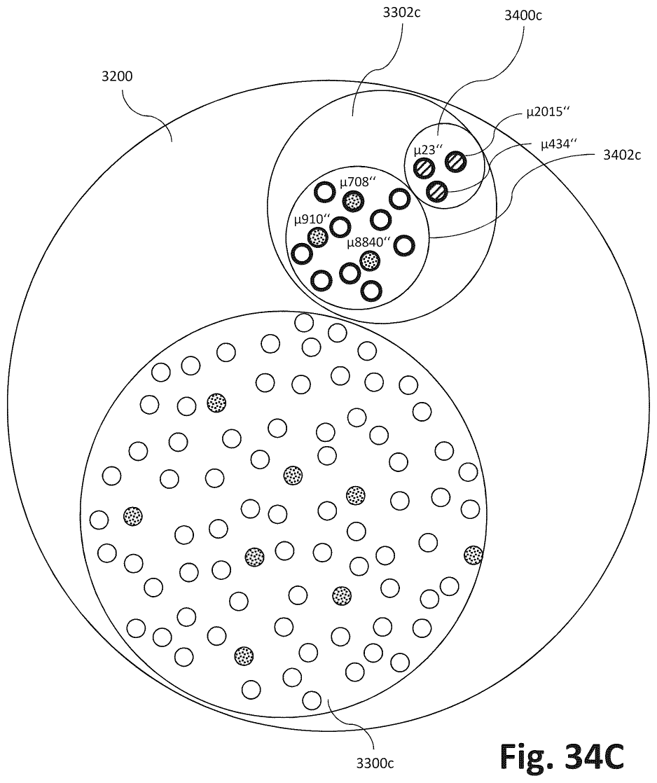

FIGS. 34A-C are to be understood as sorted sets of the plurality of sets of combinations of dyes S1, 3200 in FIGS. 33A-C, wherein the “set of combinations of dyes not subsumable under a first, second, and third readout sequence” 3300a, 3300b, 3300c and the “sets of combinations of dyes subsumable under a first, second, and third readout sequence” 3302a, 3302b, 3302c are shown. The “sets of combinations of dyes subsumable under a first, second, and third readout sequence” 3302a, 3302b, 3302c are further divided into the set of markers that are actually present (true positives) 3400a, 3400b, 3400c and the false positives which may be type I (combinations of dyes subsumable under the readout sequence not assigned to a marker) or type II (combinations of dye subsumable under the readout sequence assigned to a marker that is physically not present in the readout volume/effective PSF). FIGS. 34A-C are best observed by swiftly changing from FIG. 34A-C, which animates this example. As the example illustrates the assignment of combinations of dyes changes from one iteration to the next in a random fashion (comparable to drawing randomly with putting back). Further the composition of 3400a, 3400b, 3400c changes with respect to the combinations of dyes, but not with respect to the markers they encode. As 3400a, 3400b, 3400c is the set of true positives their associated combinations of dyes will in all cases be subsumable under the respective readout sequence. In other words the affinity reagents and their respective markers will be an element of 3400 in all iterations. But because in the three illustrated iterations, the assignment of the combination of dyes to the affinity reagents is changed, the true positives of the first readout are indicated as markers μ23, μ2015, μ434 in FIG. 34A, the true positives of the second readout are indicated as markers μ23′, μ2015′, μ434′ in FIG. 34B and the true positives of the third readout are indicated as markers μ23″, μ2015″, μ434″ in FIG. 34C. It is to be understood that the marker μ23 and μ23′ and μ23″ share the same affinity reagent but comprise a different combination of dyes. The composition of 3402a, 3402b, 3402c, which contains the set of false-positives changes substantially with every iteration both with respect to the combinations of dyes and with respect to the included markers. From one iteration to the next as shown in FIG. 34A and FIG. 34B the set of false positives may contain one or more of the same markers by chance such as μ4124, μ4124′. This means, that the decoding of multi-species readout volumes is possible with an iterative approach and that the probability of observing a false positive depends on the fraction of combinations of dyes subsumable under the readout sequences. In other words, if 3202 is a large fraction of 3200 it is more likely to observe false-positives (type I and type II) and more iterations have to be performed to gain a user-required level of statistical confidence. If 3202 is a small or extremely small fraction of 3200 as it easily may be (compare FIG. 31) then a few iterations will lead to excellent p values and statistical confidence in the detection of markers in the readout volume/effective point spread function. In other words, a smaller □□ will lead to higher confidence in a smaller number of iterations. Consequently, the cardinality of the set of combinations of dyes 3200 (which may also be named the plurality of combinations of dyes S1) fundamentally limits this approach. As the cardinality grows exponentially, while the number of interesting target molecules is fundamentally limited (e.g. ~20,000 protein coding genes), this is an extremely powerful approach to a wide array of problems in microscopy and cytometry both for life science research as well as for diagnostic applications.

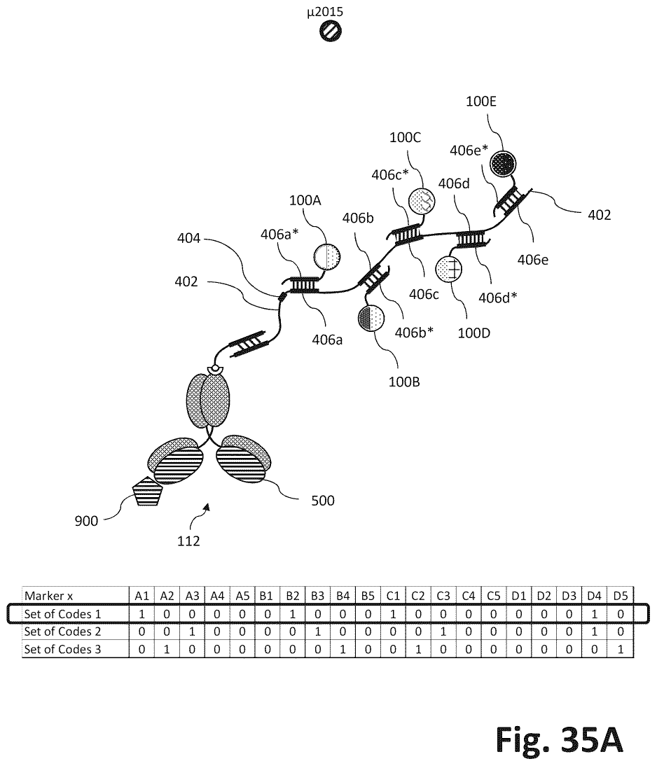

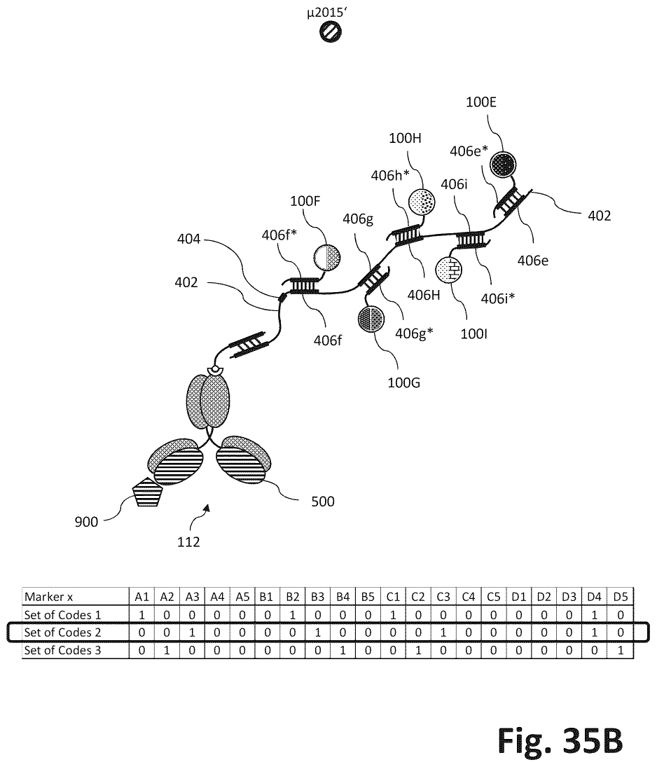

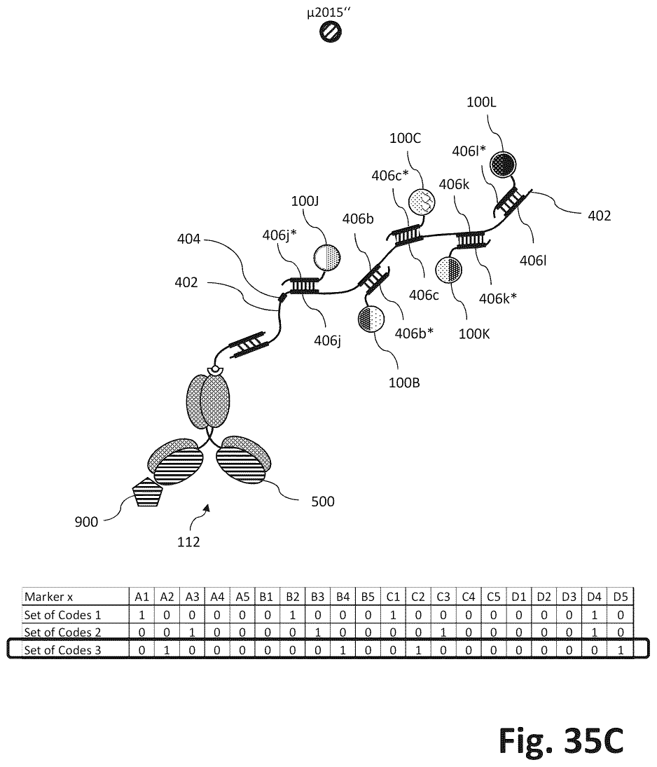

FIG. 35A to 35C show a given marker 112 with a certain reactivity 500 to a target molecule 900. In the first iteration shown in FIG. 35A a first combination of dyes is attached to the marker μ2015, in the second iteration shown in FIG. 35B a second combination of dyes is attached to the marker μ2015′, in the third iteration shown in FIG. 35B a third combination of dyes is attached to the marker μ2015″.

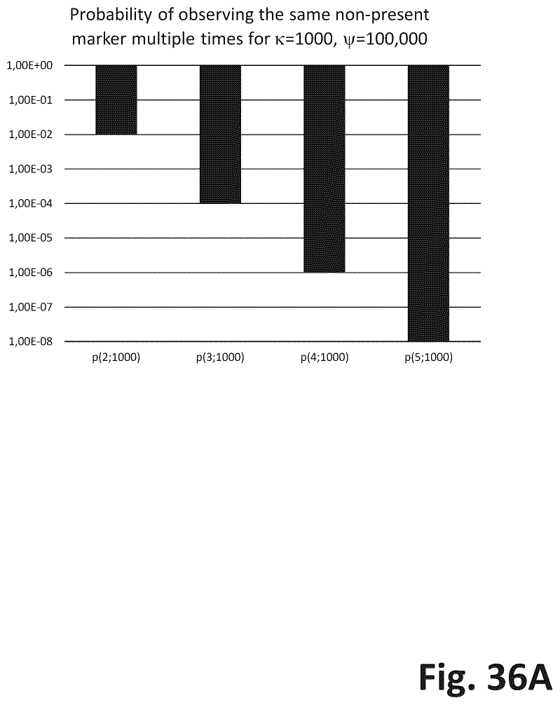

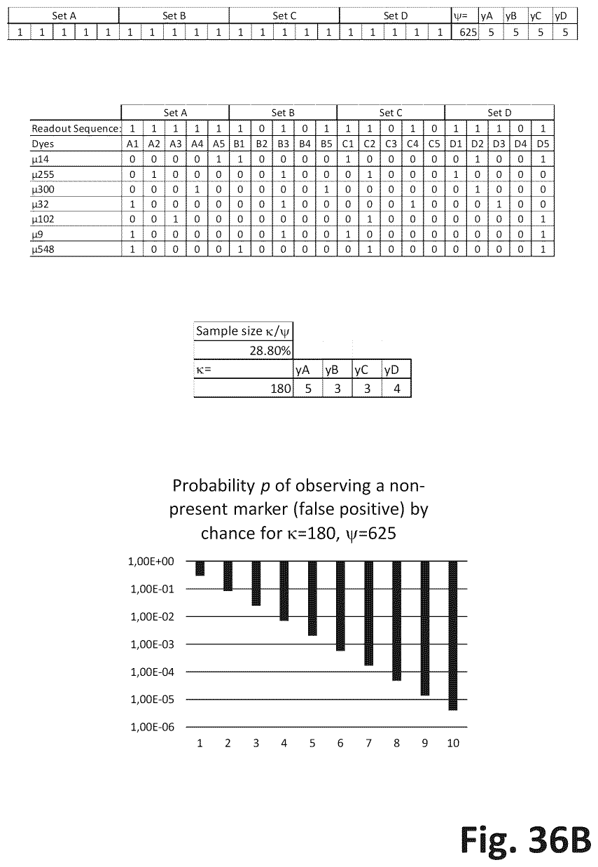

FIG. 36A is a bar chart showing the probability pi of observing the same non-present marker (type II false-positive) multiple times, which is given by □i/□, wherein □i is the □ in the i-th round. For the sake of simplicity □I is set to 1,000 in all rounds corresponding to 1% of □ in this example. The probability pi is an important factor impacting the marker-specific p values and is itself strongly dependent on and directly proportional to D.

FIG. 36B shows another example with n=4 and yA=yB=yC=yD=5 and □=625 and an exemplary readout sequence as well as a list of combination of dyes and corresponding markers subsumable under the readout sequence. Combinations of dyes subsumable, but not assigned to a marker, i.e. type I false positives, are directly excluded from the list and not shown in this example. In this example □=180 corresponding to □□28.8%. This means that in order to observe a list with relatively few markers including true positives and type II false positives one would expect that only a small fraction □ of the available codes can be used. If as in this example □ is small than this is an important practical limitation. For the attainable □□s that can be easily in the range of >104, >106, >1010, >1015, >1020 and more this becomes practically less and less relevant. In the example shown in FIG. 36B more than 10 iterations would have to be performed to bring the probability pi of observing the same non-present marker (type II false-positive) multiple times into the range of 10-6. For a small enough □ this would be attained after the second iteration.

FIG. 37 shows a workflow for primary qualitative iterative multi-species readout volume decoding. The workflow starts in step S3700. In a step S3702 the sample is stained with at least a sub plurality of markers which are labelled with a first set of combination of dyes. The first readout sequence is acquired in step S3704 and a first set of all combination of dyes subsumable under the first readout sequence is retrieved from the memory device and is itself stored in a memory device in step S3706. Now the dyes of the first iteration introduced in step S3702 are inactivated in step S3708. Next in step S3710 the sample is stained with at least the sub plurality of affinity reagents used in S3702 which are now labelled with a second set of combination of dyes (i.e. a second sub plurality of markers with the same reactivity as the first, but different labelling), the second readout sequence is acquired in step S3712 and a second set of all combination of dyes subsumable under the second readout sequence is retrieved from the memory device and is itself stored in a memory device in step S3714.

In step S3716 the first and second sets of combination of dyes subsumable under the first and the second readout sequence respectively are compared with each other and the overlap is established and stored in a memory device. In a step S3720 a statistical confidence in the decoding result is evaluated and p values and/or another suitable measure of statistical confidence are calculated for each marker in the overlap in step S3720. This may include the use of intensity information as well as include further information of multiple measurements in overlapping confocal volumes/effective point spread functions. If p values are acceptable to the user in step S3722, the processes can be ended in step S3724. Alternatively, further iterations may be performed as indicated by the arrow pointing back to step S3708.

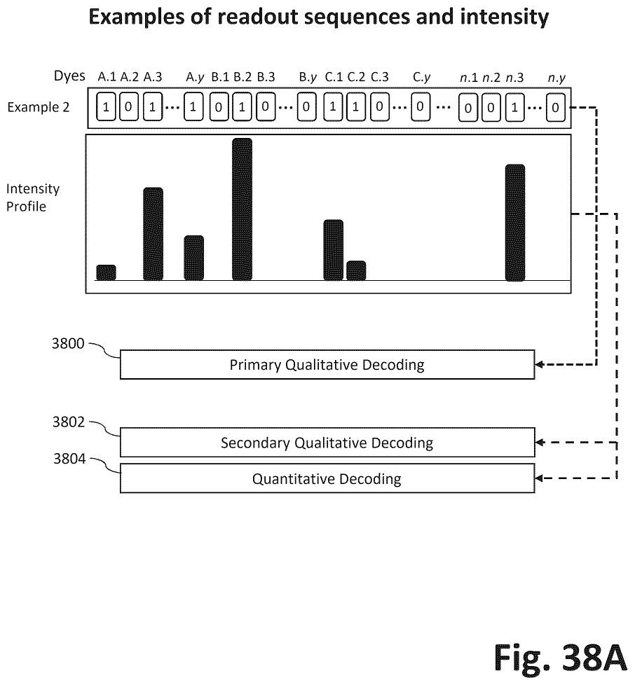

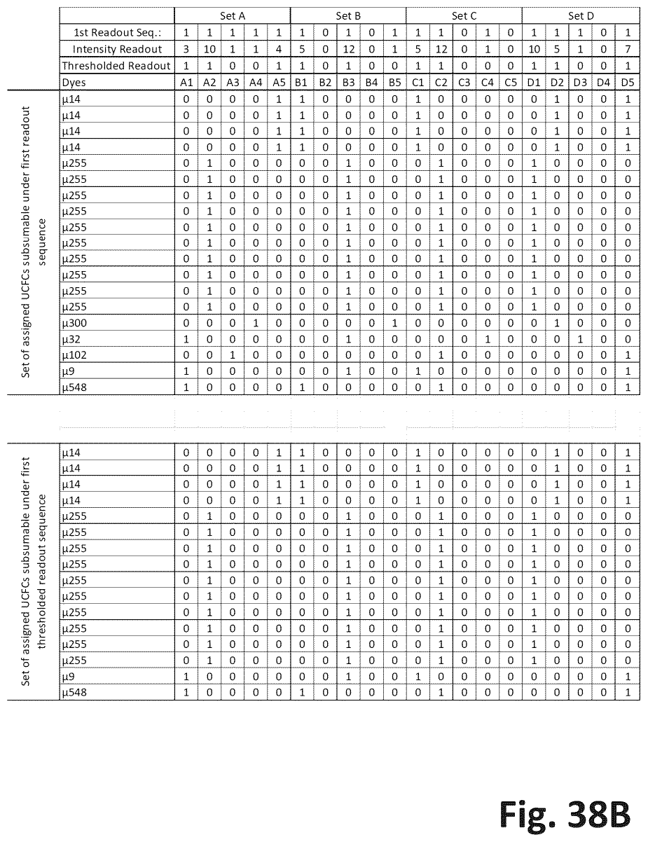

FIG. 38A shows an example of a readout sequence and an intensity profile corresponding to the intensities of the corresponding dye channel. A dye detected in the confocal volume/effective point spread function is denoted with a “1” in the corresponding digit of the readout sequence. At the same time the intensity information for each dye is stored in a memory device. As illustrated in FIG. 38A primary qualitative decoding 3800 works with multiple readout sequences obtained by the iterative process described above and against the set of combination of dyes 3200 to find multiple sets of combination of dyes subsumable under the respective readout sequences to establish an overlap and statistical confidence in the decoding result. Secondary qualitative decoding 3802 is either a decoding strategy in its own right or an additional optional step after primary qualitative decoding 3800. Secondary qualitative decoding 3802 in contrast to primary qualitative decoding 3800 takes intensity information such as the intensity profile into account. This may be done in a simple way by intensity thresholding the readout sequence, which may drive substantial increases in statistical confidence in the overall result as illustrated in FIG. 38B. Alternatively, or in addition, it may be used in a special form of linear unmixing. Essentially, this works because primary qualitative decoding 3800 defines the identities of the markers included in the confocal volume/effective PSF based on certain level of statistical significance. If this list of markers is taken as an input then the problem is a fully determined or overdetermined set of linear equations, which can be solved. This is analogous to how linear unmixing is performed, wherein the intensities for individual dyes are unmixed from overlapping emission spectra. In this case the relative number of combination of dyes is found, which is subsumable under all readout sequences and explains the shape of the intensity profile. The result of this process is a relative quantitative decoding, i.e. the combination of a certain set of readout sequences, a certain set of markers found in the confocal volume/effective PSF based on certain level of statistical significance are found to be consistent with a certain relative quantity of markers and target molecules, such as consistent with μ14 to be 4× higher than μ9 or μ548 and μ9 10× lower than μ255 as schematically illustrated FIG. 38B. If suitable calibration of the readout device is being performed using a suitable standard the method may also provide absolute number of markers and target molecules inside the confocal readout volume and the effective PSF.

FIGS. 9, 10A and 39 to 47 show various particularly preferred embodiments of markers, which generally contain an affinity reagent 112 that binds to target molecule/predetermined structure/analyte 900 with their specificity determining region, moiety, or sequence 500, which may be a paratope or a complementary sequence in case of an antibody or an oligonucleotide for example. An affinity reagent may also be a small molecule/drug/drug-like molecule/toxin 118, i.e. an affinity ligand, which is bound by an affinity receptor in the sample. The marker is connected to the linker 902 either directly through covalent conjugation or indirectly through oligonucleotides and other methods. The linker further connects to a combination of dye, i.e. a specific combination of dyes 600. The combination of linker and fluorescent dyes is referred to as reporter 908 in the sense of this document. Dyes in a combination of dye may be present in a stoichiometric amount or non-stoichiometric amount depending on the particular requirements of the application in question and the linker type and coupling or connecting strategy that is being used. The linker may be endowed with further functionalities in particular with at least one cleavage site 904, which allows the particularly easy and swift removal of the linker including the dyes. This is particularly preferable, as a way of dye inactivation. The cleavage site 904 may be a nucleotide sequence such as a restriction site, a target for a CRISPR/Cas or similar enzyme, a photocleavable linker, a proteolytically addressable site such as for example a Caspase cleavage site.

Alternatively, or in addition the linker may carry a unique oligonucleotide sequence barcode (UOSB), which identifies a certain combination of dye and can be used to attach the reporter in a flexible way to any affinity reagent carrying a complementary sequence to the respective UOSB. Using this strategy or simply attaching the reporter using a generic oligonucleotide sequence to an oligonucleotide conjugate to the affinity reagent is advantageous as it allows the flexible attaching and removing of dyes by means of hybridization and melting. Using oligonucleotides to connect antibody and linker by means of hybridization is particularly preferable, as it allows the affinity reagent to be brought into and to stay inside the sample independent of the reporter. Furthermore, once affinity reagents are bound to their target structures this strategy allows the particularly easy switching of combination of dyes as part of the iterative multi-spot decoding process as it does not necessitate the removal of the affinity reagents from the bound target.

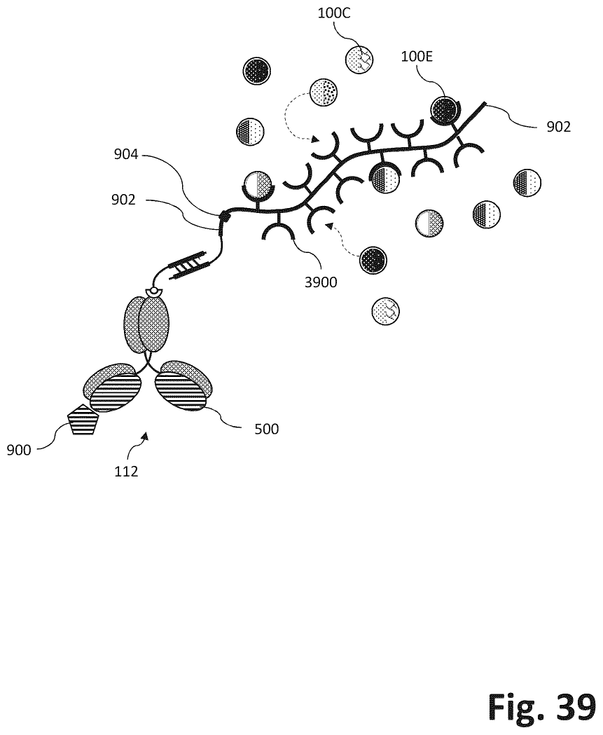

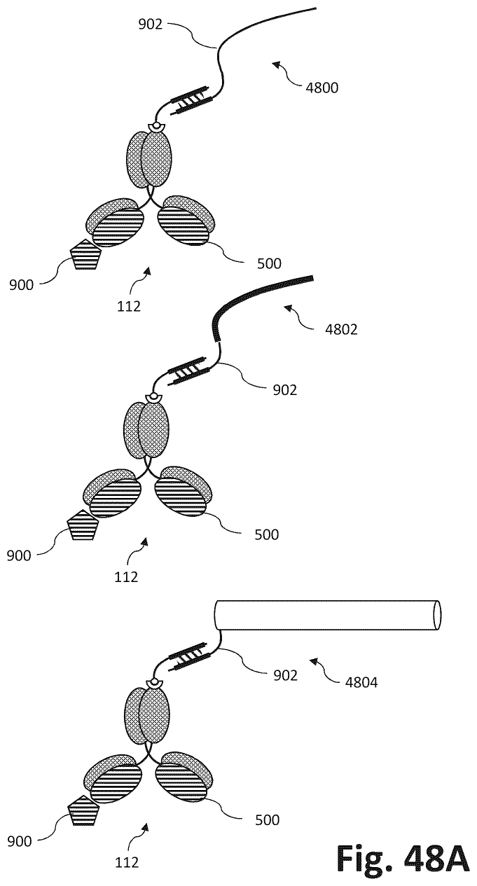

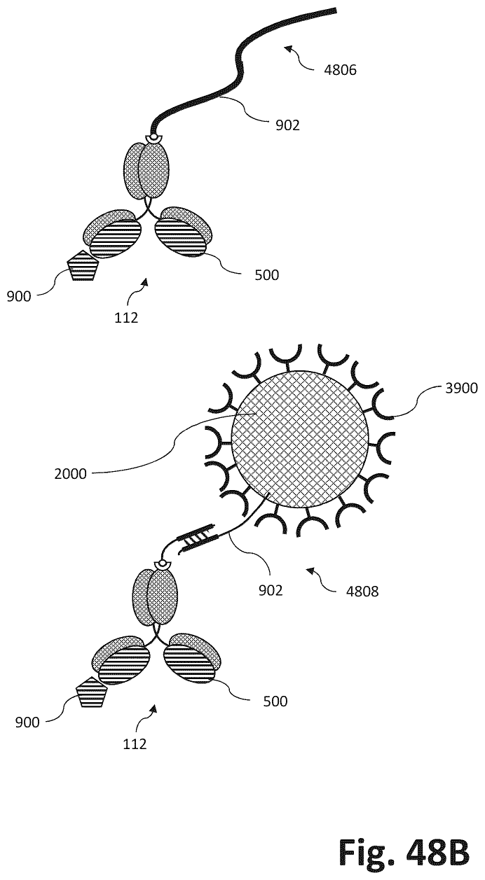

FIG. 39 shows a marker with a linker 902 comprising an oligonucleotide backbone with may also be a peptide, a nanoruler or another DNA-origami-based structured (compare FIGS. 48A and 48B) unspecific binding sites 3900 for fluorescent dyes, these may be sites for NHS- or maleimide coupling, site for click chemistry coupling such as alkine-azide coupling. In the case of FIG. 39 dyes may be coupled to the linker simply by adding a mix of dyes to a reaction mixture, which will lead to a certain distribution (non-stoichiometric coupling).

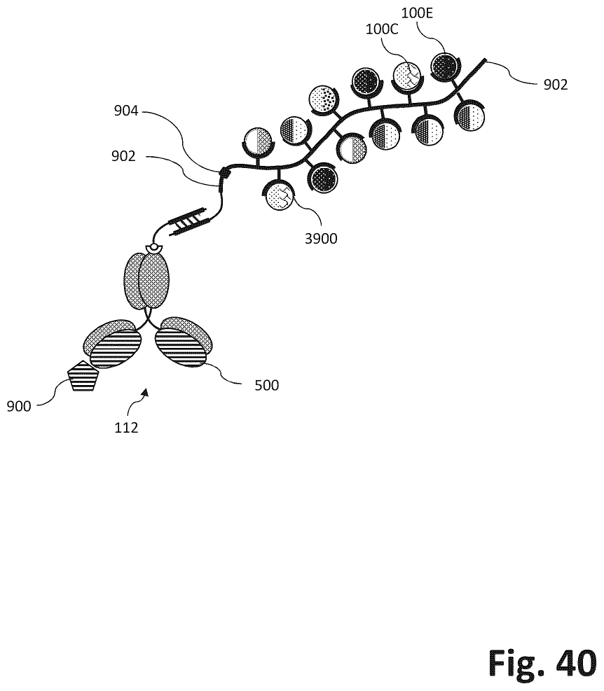

FIG. 40 shows the linker shown in FIG. 39 after the dye coupling step with all coupling sites or binding sites 3900 occupied by a dye 100C, 100E.

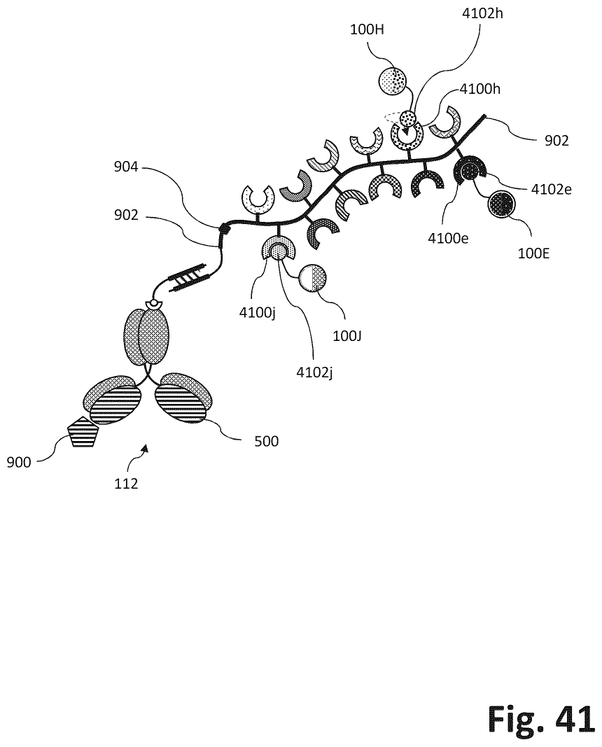

FIG. 41 shows a marker with a linker 902 comprising an oligonucleotide backbone with may also be a peptide, a nanoruler or another DNA-origami-based structured (compare FIGS. 48A and 48B), and dye-selective/specific binding sites 4100e, 4100h, 4100j for that have a specificity determining moiety or functionalization 4102e, 4102h, 4102j fluorescent dyes, these may be oligonucleotides. In this is a particularly preferable embodiment shown in FIG. 41 dyes may be coupled to the linker simply by adding a mix of dyes to a reaction mixture, which will lead to a stoichiometric coupling, this is particularly advantageous for the method as it facilitates quantitative decoding.

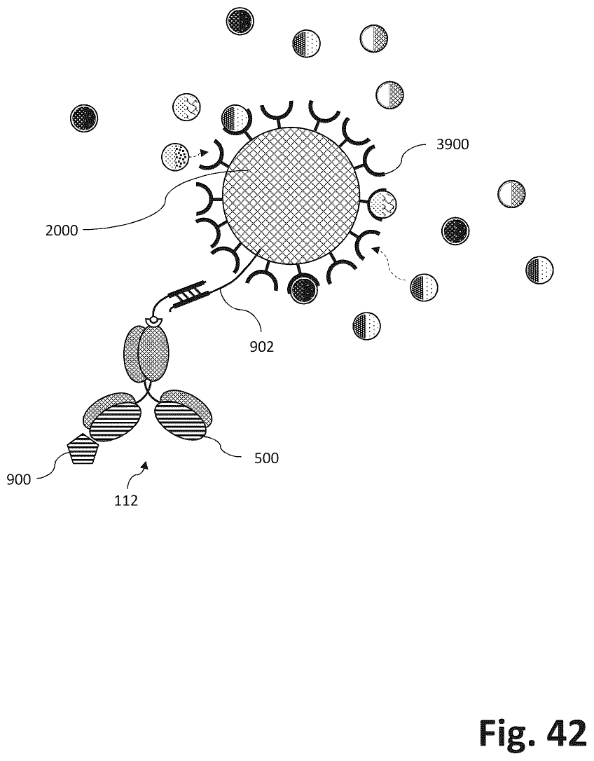

In a particularly preferable embodiment of the invention shown in FIG. 42 a bipartite linker is used consisting of a UOSB and a conjugated microbead 2000, which is functionalized with unspecific coupling sites 3900 and used as described in FIG. 39. This is particularly advantageous embodiment for assays that require higher sensitivity as a high number of dye structures or molecules can be coupled to microbead, which serves as 3-dimensional support and part of the linker.

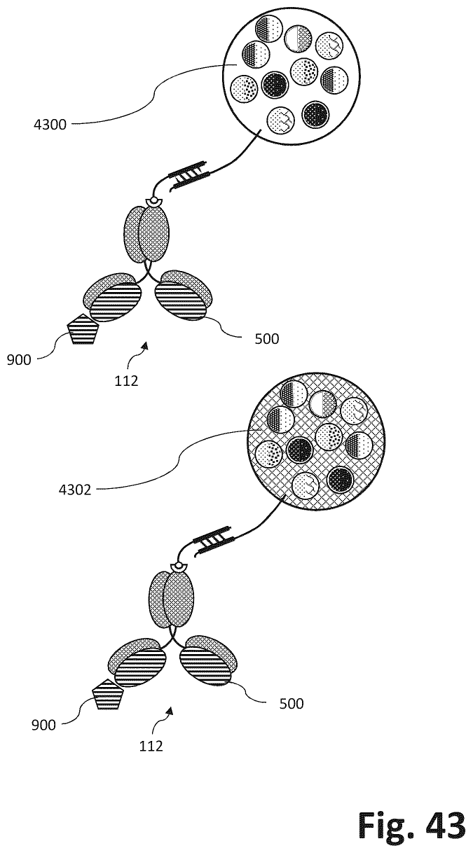

In a further particularly preferred embodiment of the invention shown in FIG. 43 dye mixes corresponding to combination of dyes are encapsulated in nano-/micro-capsules 4300 or embedded in nano-/micro-beads 4302. Such structures can be efficiently synthesized using nano-/microfluidics, electro-spraying, acoustic droplet ejection or emulsification and have the advantage that they shield the enclosed dyes from environmental influences. Relevant environmental influences are in particular ozone and other reactive oxygen species, pH value, solvents, buffers and the like. It is particularly advantageous to shield dyes for use in the disclosed methods as this stabilizes their fluorescence lifetimes and thus aids in dye separation on readout device that are configured to record, possibly amongst others, fluorescence lifetime information.

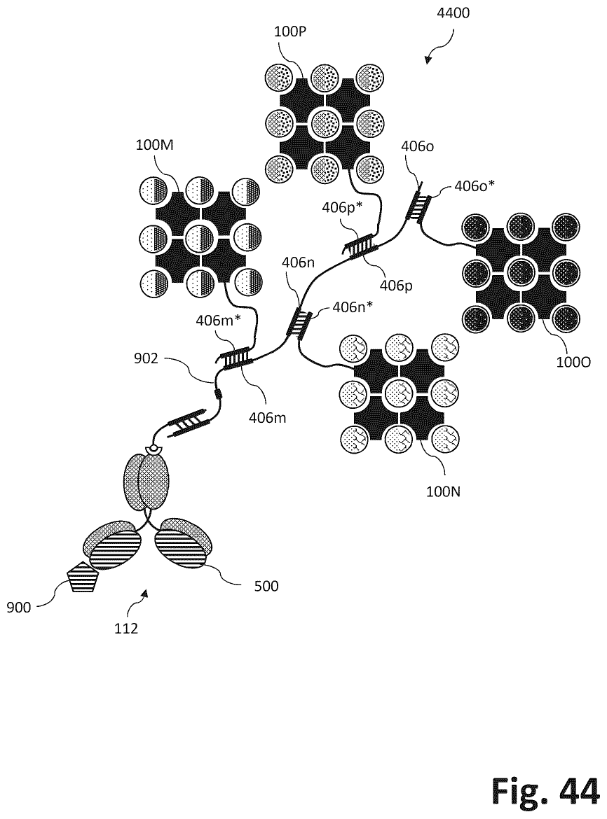

In a further particularly preferred embodiment of the invention shown in FIG. 44 the dyes used are SMILEs or small-molecule ionic isolation lattices as described by Benson et al. 2020 in Chem 6, 1978-1997. SMILEs offer the principle advantage that they allow very high dye concentration in very small volumes (1 dye per ~4 nm³), while avoiding quenching, and therefore provide very high brightness. Furthermore, the co-crystallization of cationic dyes with anion-binding cyanostar macrocycles shields the incorporated dyes from environmental influences and thus stabilizes the fluorescence lifetime, rendering SMILEs ideal for dye separation on readout device that are configured to record, possibly amongst others, fluorescence lifetime information. Furthermore, SMILEs show exceptional brightness and thus ideally suited for a range of applications that require high sensitivity. As shown in FIG. 44 SMILEs 100M, 100N, 100O, 100P may be coupled to a linker generating a SMILE-based reporter 4400.

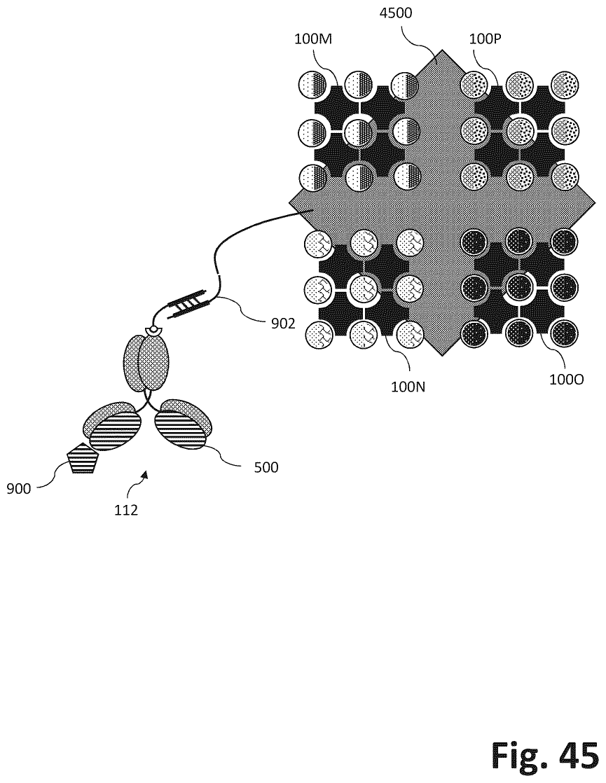

In a further particularly preferred embodiment of the invention shown in FIG. 45 SMILEs 100M, 100N, 100O, 100P are used in conjunction with a nanostructure 4500 that is used as a part of the linker like a platform or support onto which SMILEs can be coupled. The nanostructure 4500 may in particular be a DNA-origami based nanostructure (such as a nanoruler with a substantially elongated shape, or any other geometry e.g. a pyramid, a grid, a sphere, a complex structure), a graphene-based nanostructure or another form of nanostructure.

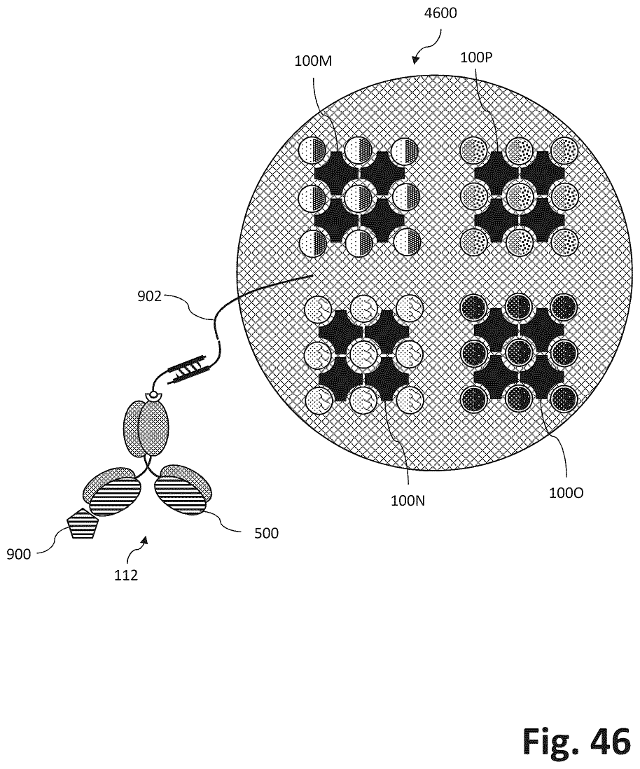

In a further preferred embodiment of the invention shown in FIG. 46 SMILEs 100M, 100N, 100O, 100P are embedded into a microbead generating another form of SMILE-based reporter 4600.

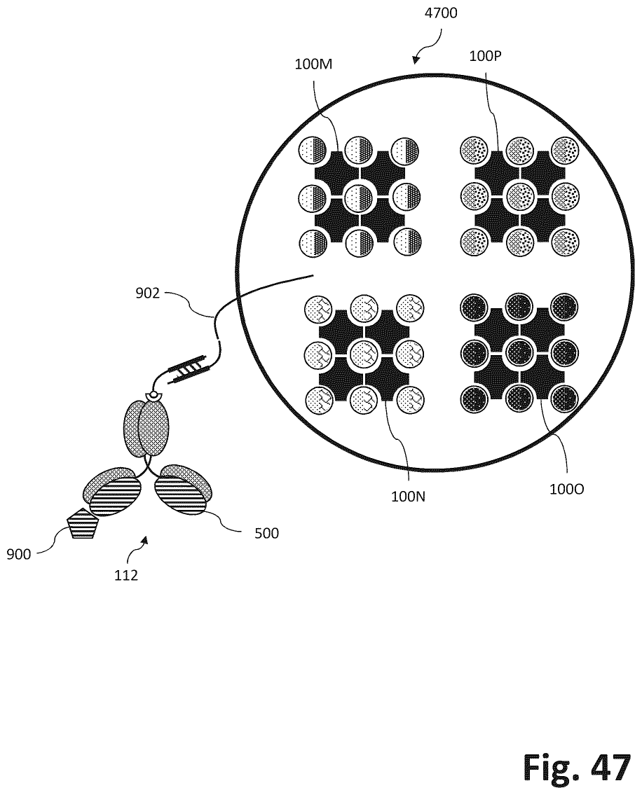

In a further embodiment of the invention shown in FIG. 47 SMILEs 100M, 100N, 100O, 100P are encapsulated into a microcapsule generating another form of SMILE-based reporter 4700.

As shown in the embodiments above reporters can be linked in various ways to the affinity reagent. FIG. 48A and FIG. 48B schematically show a collection of particularly preferable linkers such as a unipartite oligonucleotide sequence linker 4800, wherein DNA, RNA, LNA, peptide nucleic acid, morpholinos or other artificial nucleic acids may be used. This is particularly advantageous as libraries of oligonucleotides sequences can be manufactured at low cost. Further it is particularly advantageous, because complementary sequences can be used to attach dyes or the affinity reagent to the linker in a reversible fashion using the principle of hybridization and melting. A range of commonly used protocols such polymerase chain reaction, in situ hybridization, fluorescent in situ hybridization, Sanger and next generation sequencing, as well as digestion with restriction enzymes and cutting with targeted endonucleases such as CRIPR/Cas for example can be used in conjunction with oligonucleotide based linkers.

In another embodiment of the present invention the linker comprises at least a combination of an oligonucleotide and a peptide sequence and may be referred to as oligonucleotide-peptide-based linker 4802.

In another particularly preferred embodiment, the linker is a nanostructure 4500 and in particular a DNA-origami-based structure or a nanoruler and may be referred to as nanostructure-based or nanoruler-based linker 4804.

In another preferred embodiment of the present invention a peptide-based linker 4806 is used.

In a particularly preferred embodiment of the invention a linker comprising at least one nano-/microbead is used 4808.

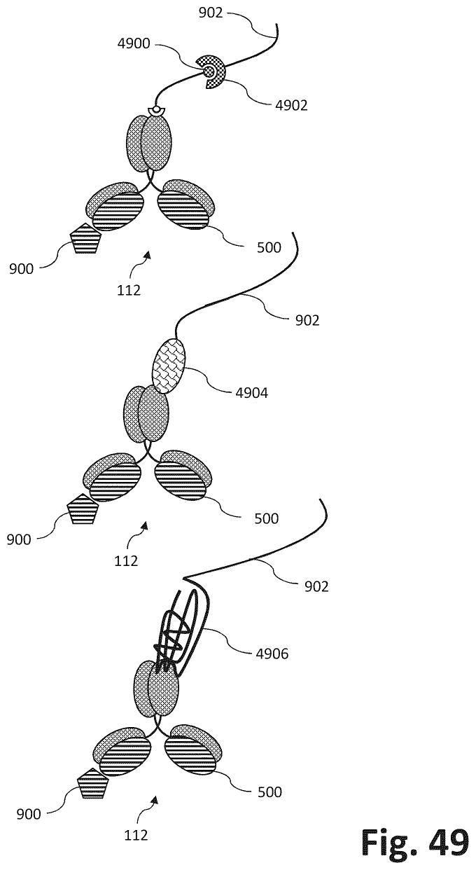

Reporters may be either directly conjugated to the affinity reagents through standard coupling chemistries such as NHS, maleimide, or various “click chemistries” such as azide-alkine coupling or they may be non-covalently linked using for instance nucleic acids and hybridization between a UOSB and a complementary sequence or a high affinity interaction between an affinity ligand 4900 and an affinity tag 4902 such as for example the biotin-Streptavidin interaction as depicted in FIG. 49. Alternatively, or in addition a secondary nanobody 4904 or other secondary antibody may be used to bind the reporter to the primary affinity reagent. Similarly, an aptamer-bound linker 4906 might be used to bind the reporter to the primary affinity reagent

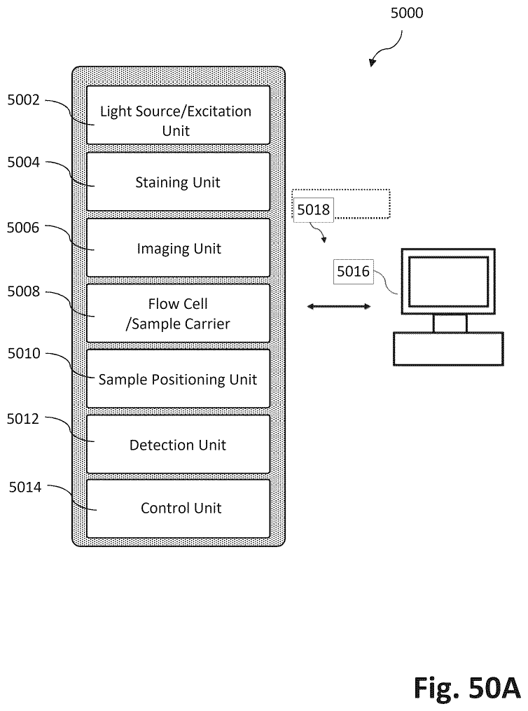

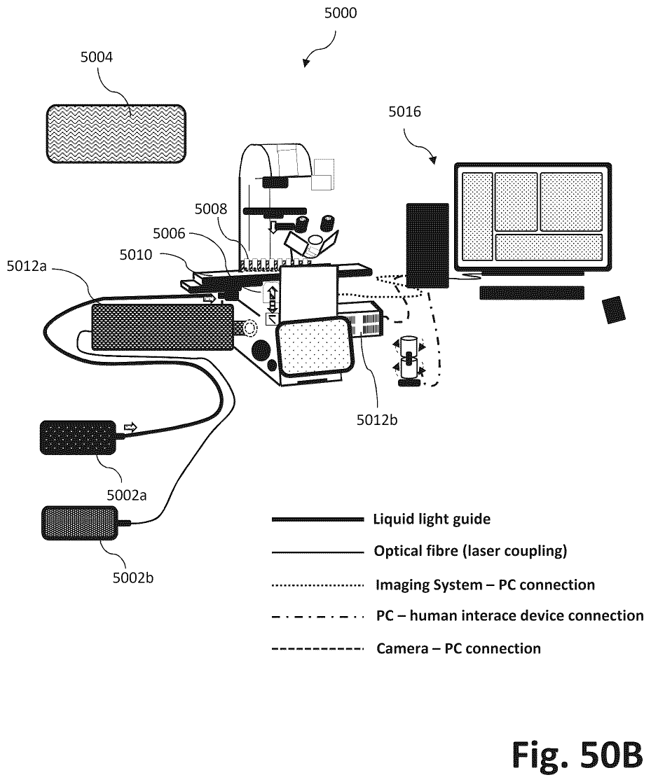

FIG. 50A shows a schematic drawing of a device 5000 for analyzing a (biological) sample. In particular, the device 5000 is capable of performing the method for analyzing a biological sample described above with reference to FIGS. 1 to 49. The device comprises at least one of the following a light source unit 5002 preferably containing LED light sources, coherent light sources (e.g. continuous wave lasers or pulsed of fixed or tuneable wavelengths, white light lasers), a staining unit 5004 for configure to perform the iterative process of FIG. 18 and related parts of the process S3702, S3710 of FIG. 37, an imaging unit 5006 in particular an imaging unit configured to record multiple views and/or optical sections, a flow cell/sample carrier 5008, a sample positioning unit 5010, a detection unit 5012, and a control unit 5014.

FIG. 50B shows a device 5000, which is a microscope or imaging system. The device 5000 may comprise a staining unit 5004 for introducing the plurality of markers 112 into the sample. For that purpose, the staining unit 5004 may comprise one or more pipettes that may or may not be automated. The staining unit 5004 may further comprise microfluidics and/or a microfluidic chip. The device 5000 also comprises an excitation unit 2314, 5002a, 5002b for exciting the fluorescent dyes 100. The excitation unit 2314, 5002a, 5002b comprises at least one light source, preferably a coherent light source. The at least one light source is configured to emit the excitation lights associated with each set of dyes. In order to emit excitation light of different wavelengths or wavelength spectra, the light source may be a tunable light source. Alternatively, the device 5000 may comprise two or more light sources with emitting light of different wavelengths or wavelength spectra. In the embodiment shown in FIGS. 23 and 24, the excitation lights emitted by the excitation unit 2314 is directed onto the sample 1500 by a beam splitting unit 2316.

An optional imaging unit 5006 of the device 5000 is configured to generate readouts which may be images or non-image-based readouts from the fluorescence light emitted by the excited dyes 100. The imaging unit 5006 comprises an objective directed at the sample for capturing the fluorescence light. The captured fluorescence light is then directed onto a detection unit 2320, 5012a, 5012b by the beam splitting unit 2316. The detection unit 2320, 5012a, 5012b comprises at least one detector element and a diffractive element 204, 206 or filters for splitting the fluorescence light into different detection channels as shown in FIG. 2.

After imaging the sample, the fluorescent dyes 100 might need to be deactivated. This can be done for example by photo bleaching the fluorescent dyes 100 with coherent light emitted by at least one of the light sources of the excitation unit 2314, 5002a, 5002b. Alternatively, a bleaching agent for chemically deactivating the fluorescent dyes 1320 can be introduced into the sample 1002 with the staining unit 5004. Further, it is possible to remove the fluorescent dye 100 from either the primary or secondary affinity reagent. This can be done for example by introducing enzymatic cleaving agent into the sample with the staining unit 5004. Alternatively, or in additionally, the fluorescent dye 100 may be deactivated by antibody elution or by dehybridization (i.e. melting) and elution in the case of fluorescently labeled oligonucleotides. Thus, the excitation unit 5002a, 5002b and/or the staining unit 5004 form a marker deactivation unit configured to deactivate at least one set of markers present in the sample.

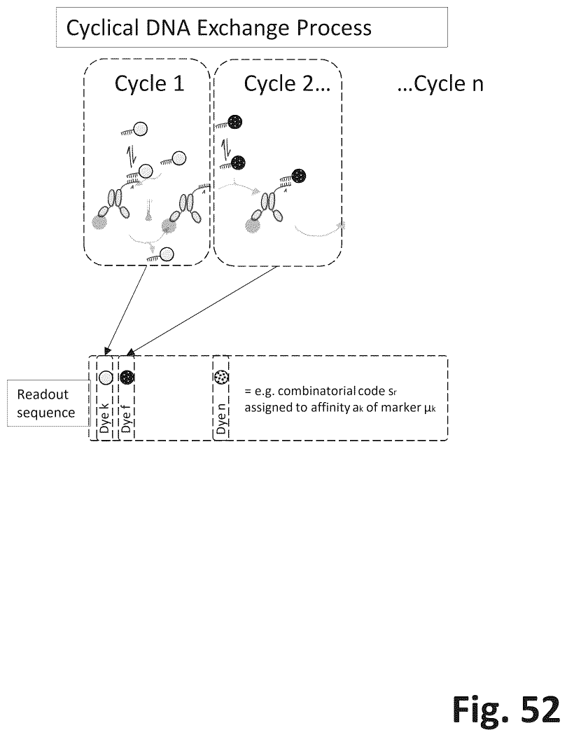

FIG. 52 schematically shows a cyclical DNA exchange process. In this example an antibody labeled with oligonucleotide-barcode contacts an analyte. In a first cycle of a cyclic staining, imaging, inactivation process a first label comprising dye k is associated with said antibody. Using DNA Exchange this first label is removed after the readout and a second label comprising dye f is allowed to attach in cycle 2. This process is repeated n times and in the n-th round dye n is readout generating a readout sequence of dye for said affinity reagent of k, f, . . . , n, which may be used to encode the identity of said affinity reagent. In other words inter-round dye-swapping is performed to encode the affinity reagent and its target analyte. The process therefore relies on repeated rounds of staining, imaging, label removal. DNA Exchange, DNA-Paint, and Exchange-Paint are related methods that may be used to label the same analyte repeatedly with different imager strands comprising different dyes in a cyclic fashion shown in FIG. 52 or in a stochastic fashion, wherein the latter may or may not require external stimuli to dissociate an associated imager strand from a cognate docking strand conjugated to an affinity reagent. These methods are described e.g. in Agasti et al. 2017 (DNA-barcoded labeling probes for highly multiplexed Exchange-PAINT imaging Chem. Sci., 2017, 8, 3080).

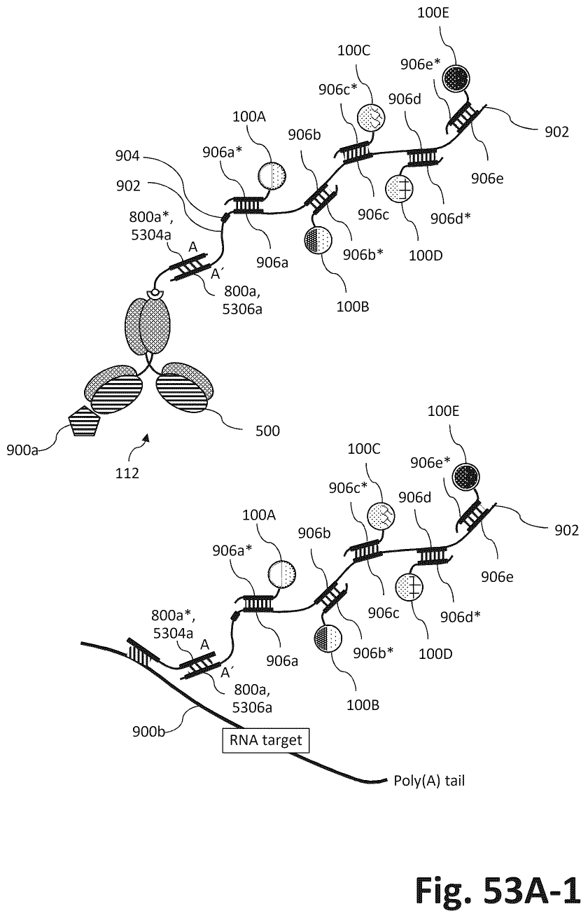

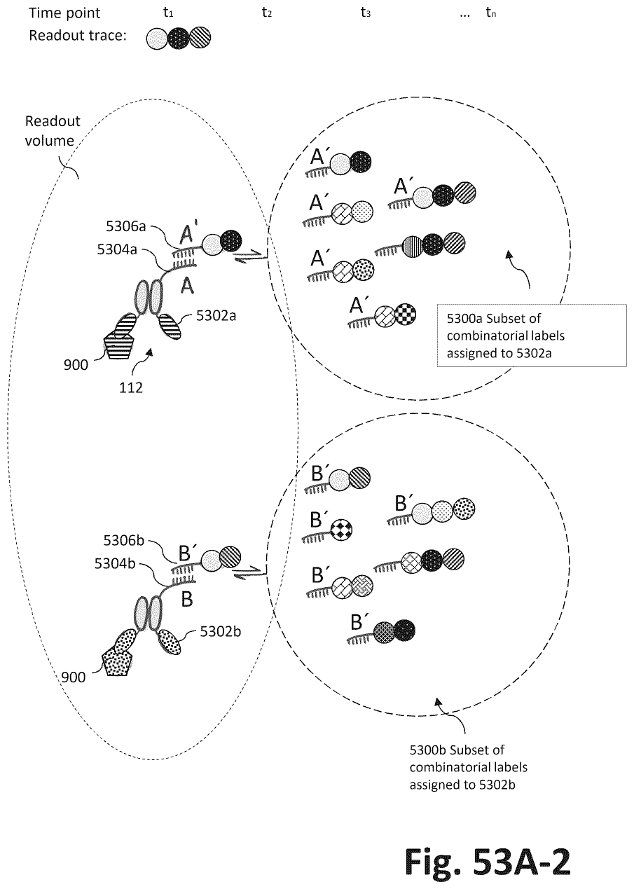

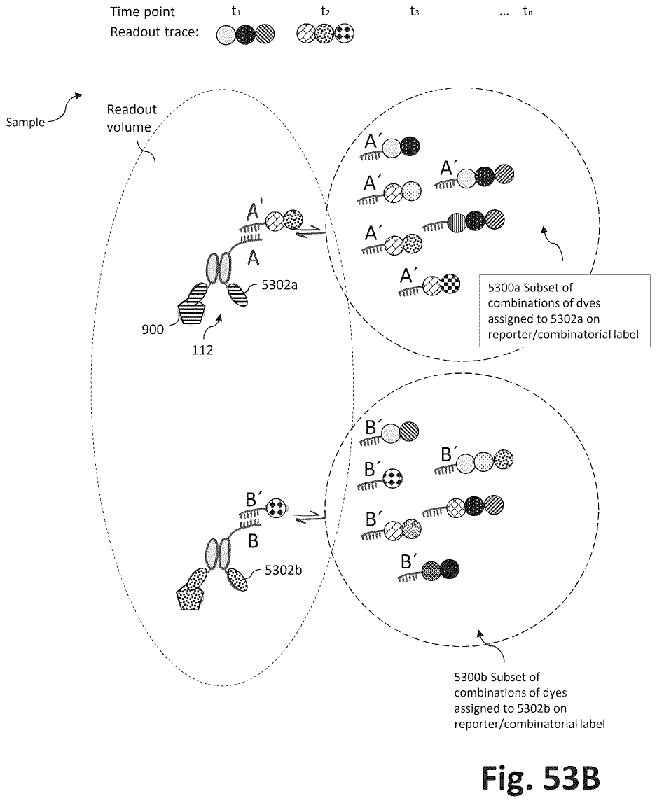

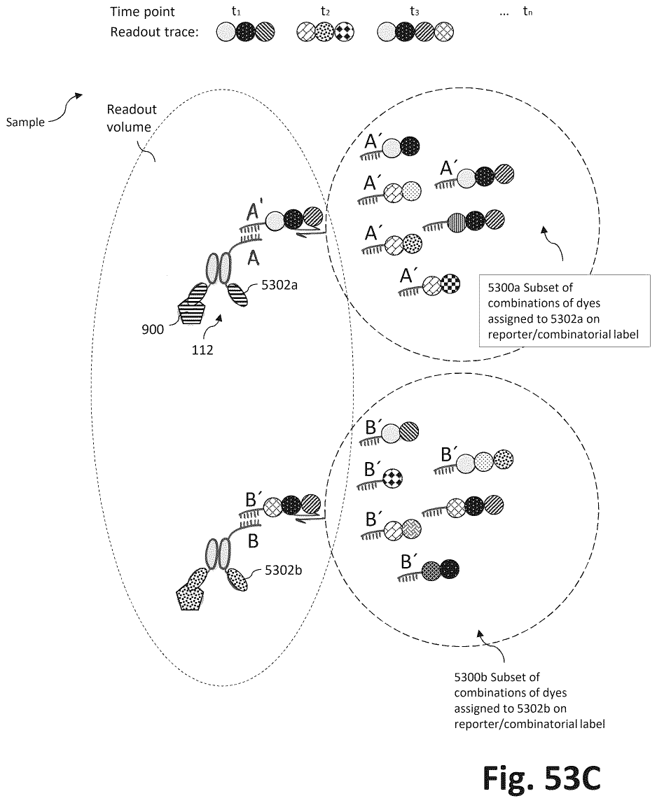

FIG. 53A-1 schematically shows different markers comprising analytes and combinatorials labels that are connected.

FIG. 53A-2 schematically shows a readout volume and a readout trace.

In contrast to the strategy depicted in FIG. 52, a “subset of combinatorial labels” 5300a, 5300b comprising a “subset of combinations of dyes” is assigned to a particular affinity reagent 5302a, 5302b. The “combinatorial labels” of “subset of combinatorial labels” 5300a comprise an “oligonucleotide attachment portion” 5306a/“A′”, which is configured to bind to the “oligonucleotide barcode” 5304a/“A” of the affinity reagent 5302a. The “combinatorial labels” of “subset of combinatorial labels” 5300b comprise an “oligonucleotide attachment portion” 5306b/“B′”, which is configured to bind to the “oligonucleotide barcode 5304b”/“B” of the affinity reagent 530ba. A and A′ as well as B and B′ are configured such that they allow either transient binding for dynamic intra-round code swapping or stimulated binding/unbinding for repeated intra-round code swapping. This means that “combinatorial labels” of subset 5300a which comprise “oligonucleotide attachment portion” 5306a “A′” can associate in a stochastic way either dynamically or repeatedly (stimulated by e.g. changing a parameter such temperature, illumination, concentration of host molecules or NCDA) with “oligonucleotide barcode” 5304a “A”. Likewise, “combinatorial labels” of subset 5300b which comprise “oligonucleotide attachment portion” 5306a “B′” can associate in a stochastic way either dynamically or repeatedly (stimulated by e.g. changing a parameter such temperature, illumination, concentration of host molecules or NCDA) with “oligonucleotide barcode” 5304b “B”.

A′ and B′ are therefore either fully or partially complementary to A and B respectively, but may include mismatches, or hairpins, i.e. stretches that do not hybridize. A′ has an affinity to A and binds to A under at least one condition transiently, i.e. for at least so long as to enable the detection of a bound reporter, before it dissociates again. The dissociation may depend on an additional chemistry like host-guest mediated ligand-invasion, strand displacement, or the addition of a special non-natural bases and further chemicals, which can be photoswitched to enable photochemical control of the hybridization of A′ to A in a reversible manner. The oligonucleotides A, A′ and B, B′ may therefore include non-natural bases and/or additions to natural or non-natural bases, such as for example the addition of a guest molecule, which is attached to a nucleobase and configured to complexate with a host molecule such as CB[7]. The oligonucleotides A, A′ and B, B′ may in addition or alternatively comprise GG mismatches, which allow the association of A and A′ or B with B′ only in the presence of sufficient concentration cis-NCDA which can be obtained from trans-NCDA by photoswitching using 360 nm and 430 nm illumination for cis-trans-isomerization.

It is important to note that it is not necessary to wash in between acquiring the readout sequences of time points t1, t2, . . . , tn which collective represent a readout trace. The binding can be detected, because the reporter or combinatorial label will associate with the respective affinity reagent for a time, which is long enough to allow detection. Both exposure time/pixel dwell time and the residence time of the reporter on the barcode of the affinity reagent can be tuned in various ways.

FIGS. 53A-1, 53A-2, 53B, 53C then illustrate changing associations between affinity reagents 5302a and 5302b with combinatorial labels from their assigned subsets 5300a and 5300b. The circles in the FIG. 53A-B filled with distinct patterns reflect different dyes from the plurality of dyes.

Referring to FIGS. 53B and 53C: A′ and B′ are complementary to A and B respectively, but may include mismatches, or hairpins, i.e. stretches that do not hybridize. A′ has an affinity to A and binds to A under at least one condition transiently, i.e. for at least so long as to enable the detection of a bound reporter, before it dissociates again. The dissociation may depend on an additional chemistry like host-guest mediated ligand-invasion, strand displacement, or the addition of a special non-natural bases and further chemicals, which can be photoswitched to enable photochemical control of the hybridization of A′ to A in a reversible manner.

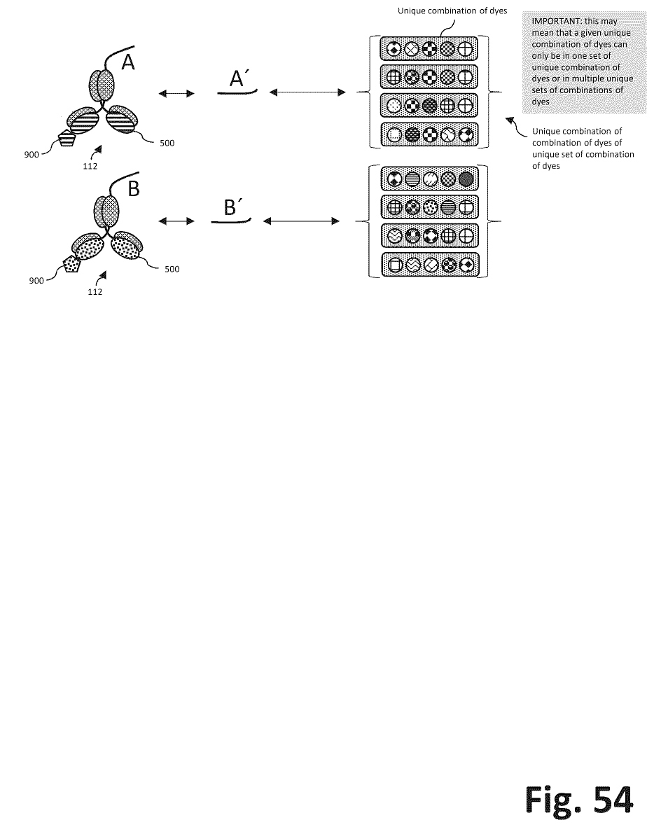

FIG. 54 further schematically illustrates the assignment of two affinity reagents to a subset of combinations of dyes from the plurality of combinations of dyes, wherein each combination of dyes in said subset can bind to the affinity reagent via the respective complementary linker A′ or B′ to the respective oligonucleotide-barcode A or B on the affinity reagent, wherein A′ and B′ may be fully or partially complementary (i.e. contain mismatches, hairpins, and the like so as long they remain suitably configured to bind to their respective attachment site or oligonucleotide-barcode connected to the affinity reagent).

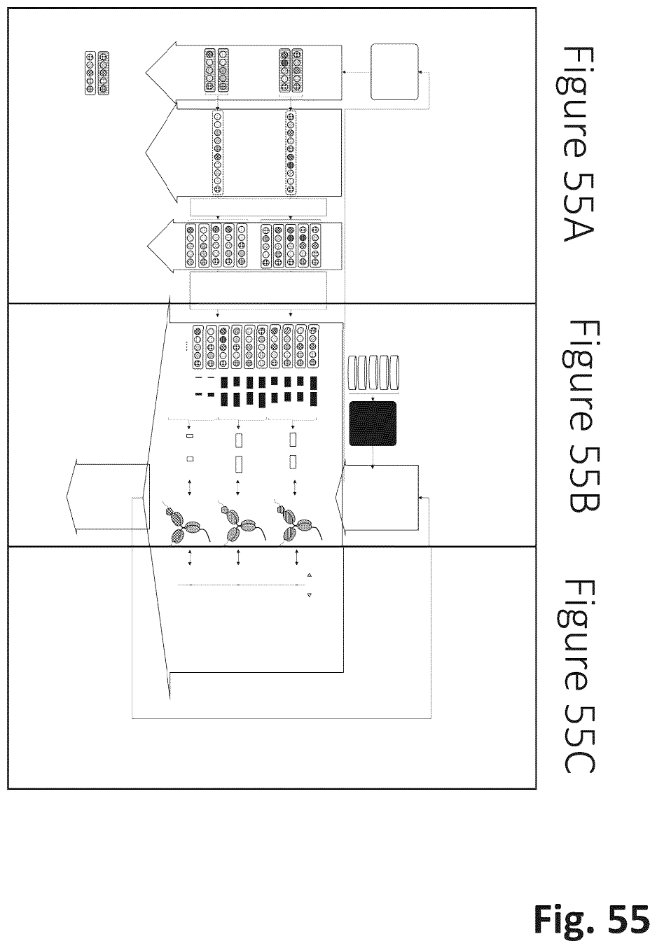

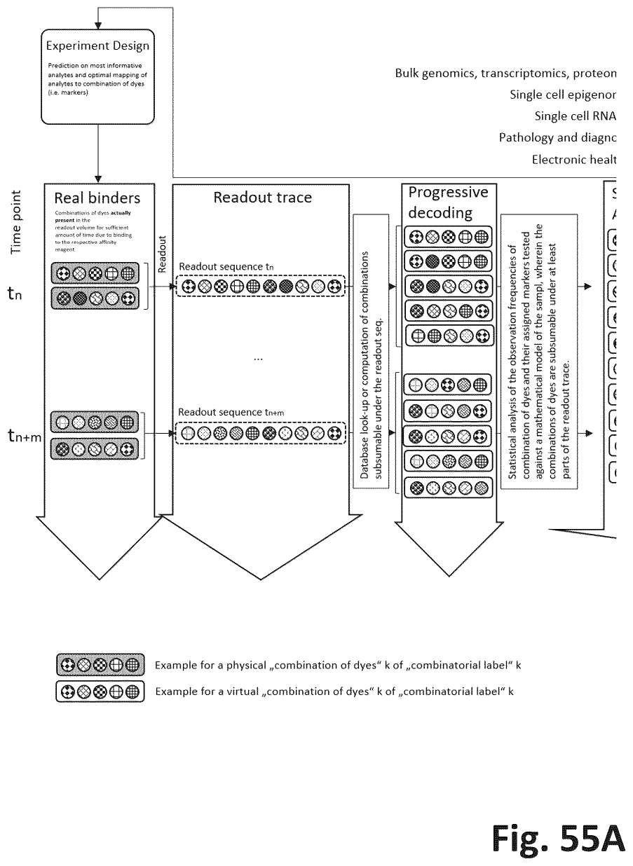

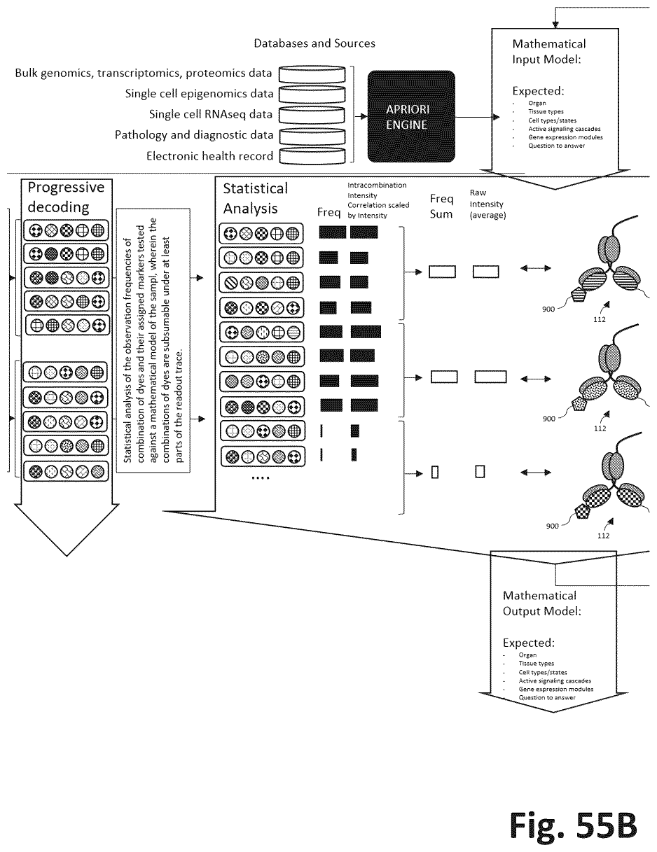

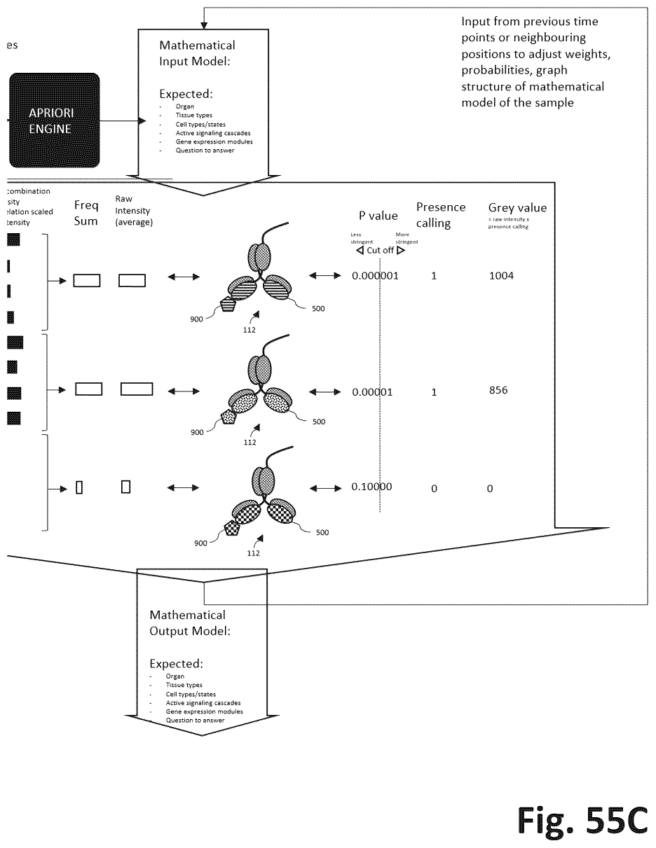

FIG. 55 shows an overview of the FIGS. 55A, 55B and 55C on the following pages, the content of the FIGS. 55A, 55B and 55C being arranged as a complete Figure as indicated in FIG. 55. FIG. 55 is a schematic overview that includes the most important entities and steps in this process/workflow and illustrates the concept of gathering readout traces, which are then used to progressively decode the sample by means statistical analysis of combinations of dyes that are subsumable under at least part of the readout trace, i.e. that may be present in the readout volume, using parameter such as the frequency, dye intensity and dye-dye intensity correlation, frequency sums, and average intensity values that are compared against a mathematical model of the sample to calculate a measure of statistical confidence in the presence of a particular affinity reagent/analyte in the readout volume, such as a p value. Presence calling may then be easily performed by a user- or system-defined global or dynamically adjusted p value cut off, wherein affinity reagents/analytes “called present” are assigned the value “1” and the ones that do not pass the threshold(s) assigned “0”. A grey value in an image may for example be derived from an average or weighted sum of all intensities belonging to all combinations of dyes readout that are assigned to particular affinity reagent and then simply multiplied with the value for presence calling “1” or “0”, which effectively cuts off noise from false-positives or true-positives that were not detected with sufficient confidence. Importantly, the APRIORI ENGINE shown in FIG. 55B can be used to load the mathematical model with inputs from various preexisting data as detailed in the European Patent application with the application number EP 22179854.9, the complete content of which is incorporated by reference herein, which may greatly improve statistical analysis in the sense of Bayesian statistics. Likewise, results from previous time-points and/or neighboring regions in the sample can be regarded a priori information that may be fed into the mathematical input model. Finally, a mathematical output model is generated, which may be an image or graph-based representation of the biological sample for instance.

As shown in FIGS. 55A-55C, from a readout trace, which effectively consists of a sequence of readout sequences collected over multiple time points tn−tn+m, a set of combinations of dyes is retrieved either from a database or computed using a hash function to generate a set of all combinations of dyes that can be subsumed under the readout trace. This represents all the possible combinations of dyes belonging to combinatorial labels that may be present in the readout volume. Said set is then statistically analysed. While some combinations of dyes subsumable under a readout trace may be easily excluded, because they either do not point to an assigned affinity reagent in the database for example. There may be a number of false-positives, which cannot be directly excluded, the frequency of retrieval of a certain affinity reagent and thereby analyte, the frequency of individual unique combinations of dyes pointing to said analyte, as well as the intensities of dyes and their correlation as well as how well their correlation fits with the combinations of dyes, can be used for statistical analysis. An aggregated observation frequency, a raw intensity value, for example an average, sum, or weighted averaged of a particular analyte and affinity reagent as well as a p value may be computed. The p value or other measure of statistical confidence indicates, with which probability the respective analyte would have been detected by the method despite the fact that it is not actually present in the readout volume (false-positive). A less or more stringent cut-off value or threshold may be applied on the p value or other measure of statistical confidence for presence calling, wherein the presence is accepted by assigning a “1” and rejected by assigning a “0”, for example. An intensity value for display in an image (grey value) may be simply computed by multiplying the raw intensity with the presence calling value, effectively netting all analyte contributions that are not statistically significant, which reduces background noise.

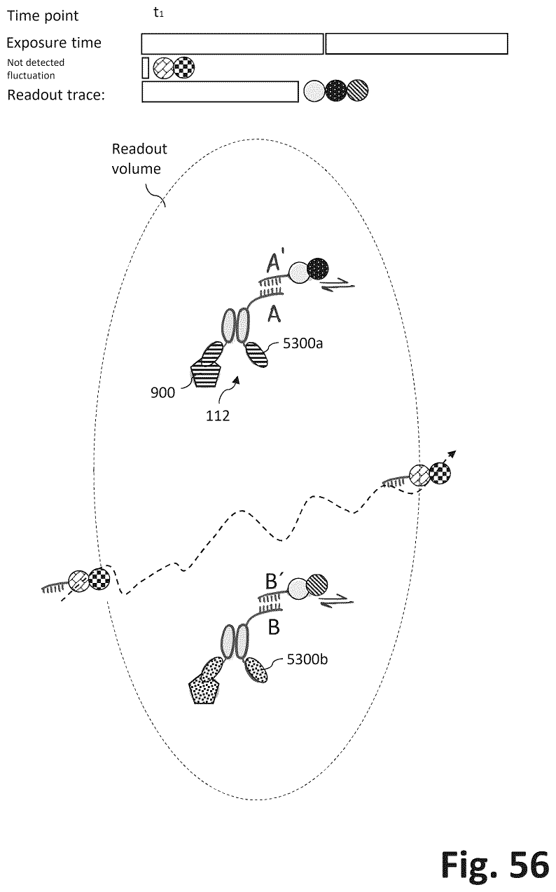

FIG. 56 schematically illustrates that unbound reporters and their combinations of dyes do not reside in the readout volume long enough to be detected as indicated by the length of bars resembling exposure/dwell time and different residence times of unbound and bound reporters.

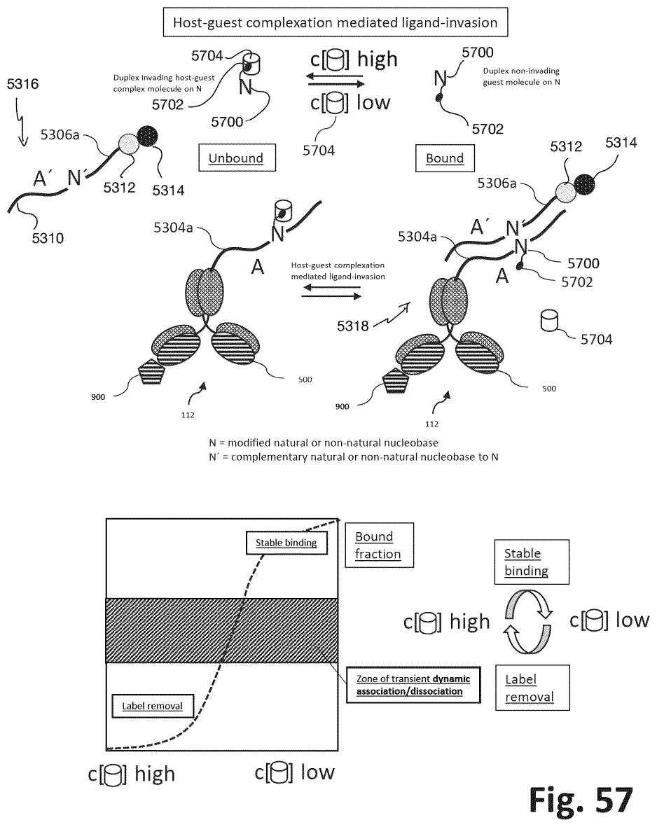

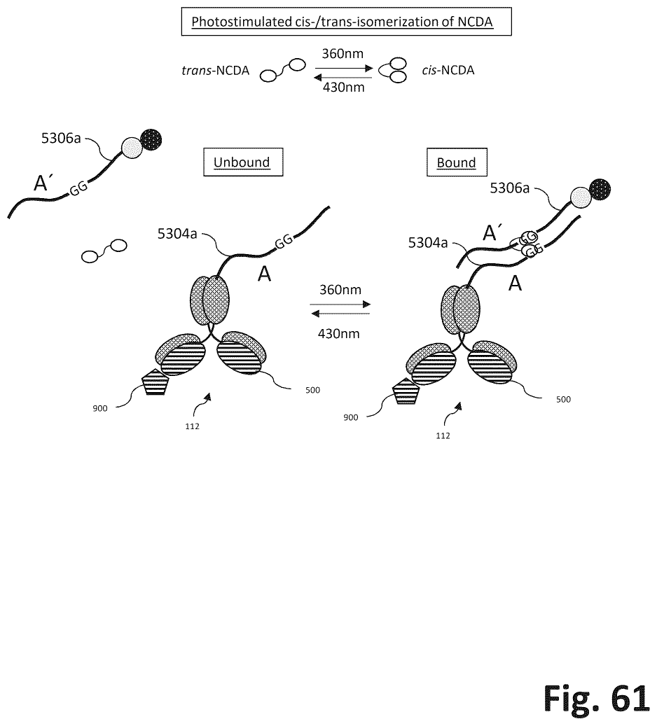

FIG. 57 and FIG. 61 schematically illustrates two strategies to bring about reversible/repeated association/dissociation or dynamic association/dissociation.

FIG. 57 shows another strategy wherein a modified DNA oligonucleotide is used that bears a modified nucleoside (“N”) 5700 harbouring a guest molecule 5702, which can complex with a host molecule 5704 like cucurbit[7]uryl (CB[7]), triggering ligand-invasion and DNA duplex dissociation. For a 15 bp duplex, 1 μM CB[7] is enough to lower the melting temperature Tm by ~9.7° C. according to Xiao et al. (Xiao, L., Wang, L L., Wu, C Q. et al., Controllable DNA hybridization by host-guest complexation-mediated ligand invasion. Nat Commun 13, 5936 (2022)). A suitable concentration of CB[7] or another suitable host molecule may be used in combination with the “oligonucleotide attachment portion” 5306a/“A′” with at least one N-guest nucleobase 5700 and the “oligonucleotide barcode” 5304a comprising at least one complementary N′ nucleobase. Alternatively, a suitable concentration of CB[7] or another suitable host molecule may be used in combination with the“oligonucleotide attachment portion” 5306a “A′” with at least one complementary N′ nucleobase and the “oligonucleotide barcode” 5304a comprising at least one N-guest nucleobase. In this way, the dynamic association/dissociation (binding/unbinding or removal) can be achieved at low temperatures by keeping the concentration of the host molecule in a suitable range, where binding and unbinding are at around 50:50. The cycling between stable binding and unbinding or label removal can be achieved by oscillating the concentration of the host molecule. This may be achieved by continuously feeding from two buffer reservoirs, one buffer reservoir with a high and the other with a low or no concentration of host molecule.

As shown in FIG. 57, the oligonucleotide attachment portion 5306a/A′ is part of a linker 5310 which is configured to bind to the affinity reagent 112. The linker 5310 comprises at least one binding site configured to bind to at least one dye. In the example of FIG. 57, two dyes 5312, 5314 are bound to the linker 5310. The linker 5310 and the at least one dye 5312, 5314 form a reporter 5316. The reporter 5316 and the affinity reagent 112 form a marker 5318 generally referred to as 5318 in FIG. 57.

FIG. 57 includes a diagram illustrating how association/dissociation depends on the concentration c of the host molecule. According to the example shown in FIG. 57, association is low in a range where the host molecule concentration c is high. In contrast, association is high in a range where the host molecule concentration c is low. Between these ranges of low and high concentrations, a transient zone exists where association and dissociation are essentially in balance. Accordingly, the transient zone separates a low concentration zone where label removal occurs from a high concentration zone where stable binding occurs.

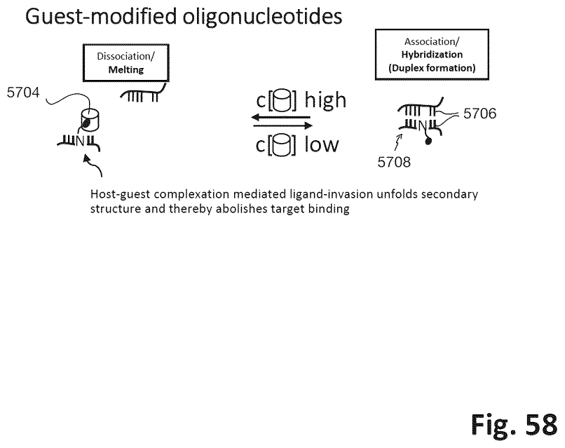

As illustrated in FIG. 58 in more detail, the host-guest complexation mediated ligand-invasion described above unfolds the secondary structure and thereby abolishes the target binding of the label. As shown on the left of FIG. 58, at a high host molecule concentration, upon unfolding of the secondary structure caused by host-guest complexation, base pairs 5706 of a duplex 5708 virtually break up, preventing duplex formation at high host molecule concentration. As a result, melting occurs at high host molecule concentration. In contrast, as shown on the right of FIG. 58, at low host molecule concentration, duplex formation due to hybridization is not prevented by host-guest complexation.

The use of guest molecule-modified oligonucleotides, as described above, allows for a relatively large oligonucleotide length while significantly reducing the melting temperature. This solution therefore has an advantage over other methods such as DNA-Exchange, DNA-PAINT or Exchange-PAINT, which use smaller oligonucleotide lengths. It can be approximated that the use of guest molecule-modified oligonucleotides proposed herein achieves a reduction in melting temperature at an oligonucleotide length of 20 to 24 mer, for which other methods operate at a significantly smaller length of 9 mer to 13 mer. On the one hand, due to their length, the guest molecule-modified oligonucleotides allow staining of the target molecules with high specificity. On the other hand, due to the low melting temperature, they allow easy inactivation of the labels without background noise becoming too strong.

Host-guest complexation mediated ligand-invasion is therefore a suitable strategy to implement “barcode cycling” and “intra-round code swapping”. The use of guest-host complexation mediated ligand-invasion as a strategy for controlling DNA hybridization is described in Xiao, L., Wang, L L., Wu, C Q. et al. Controllable DNA hybridization by host-guest complexation-mediated ligand invasion. Nat Commun 13, 5936 (2022). https://doi.org/10.1038/s41467-022-33738-3).

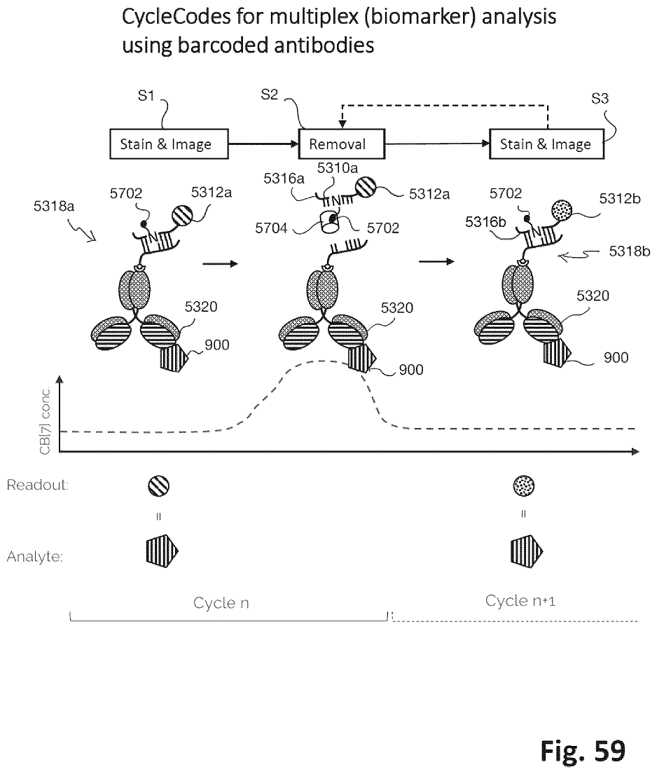

FIG. 59 shows an example for multiplex analysis using barcoded antibodies. For this purpose, markers may be used as described above with reference to FIG. 57. According to the example shown in FIG. 59, it is assumed that each marker 5318a/b comprises only one dye 5312a/b at a given time. Further, the marker 5318a/b comprises an affinity reagent 5320 formed from an antibody which is configured to attach to the analyte 900.

In a first step S1, the sample is stained with the marker 5318a that is formed from the reporter 5316a including the linker 5310a and the dye 5312a bound thereto. After staining, the sample is imaged to create a first readout. While performing step S1, the concentration of the host molecule 5704 such as CB[7] is kept low, as this is shown in the diagram in the lower part of FIG. 59. As a result, hybridization/duplex formation occurs, and the analyte 900 is labeled with the dye 5312a.

In step S2, the concentration of the host molecule 5704 is increased so that the duplex breaks up due to dissociation. As a result, the reporter 5316a including the dye 5312a is removed from the analyte 900.

In step S3, the sample is stained again. However, a marker 5318b is used that comprises a reporter 5316b having a dye 5312b which is different from dye 5312a applied in the step S1. After staining, the sample is imaged to create a further readout. While performing step S3, the concentration of the host molecule 5704 is low again. As a result, hybridization/duplex formation occurs, and the analyte 900 is labeled with the dye 5312b.

The steps of staining the sample, imaging the sample and removing the labels can be repeated to implement a cyclic process.

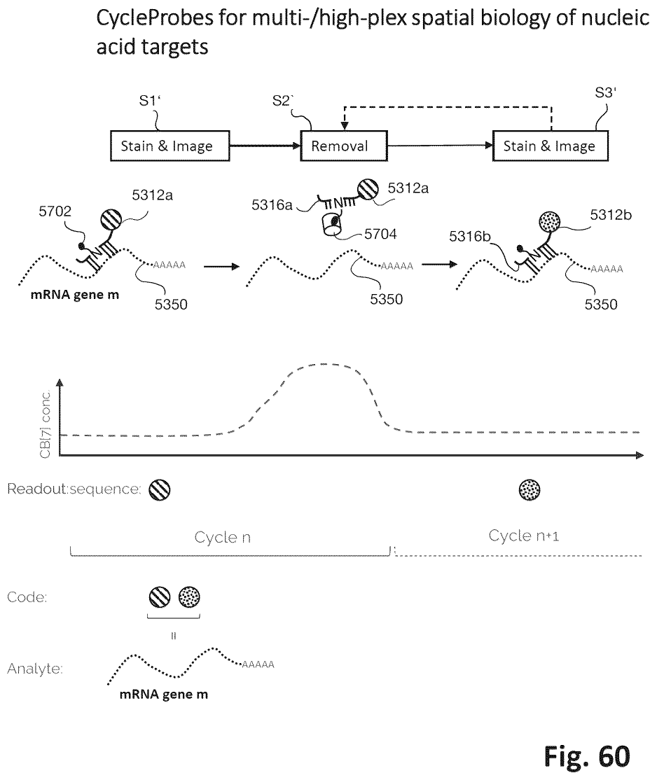

FIG. 60 shows a modified cyclic process in which oligonucleotide probes are used as affinity reagents rather than antibodies. Each oligonucleotide probe may be formed from a reporter corresponding to the reporter 5316 described above. More specifically, in this case, the reporter 5316 may be adapted for binding to a nucleic acid analyte 5350 such as a mRNA gene.

In a first step S1′, the sample is stained with the oligonucleotide probe that is formed from the reporter 5316a including the dye 5312a bound thereto. After staining, the sample is imaged to create a readout. While performing step S1, the concentration of the host molecule 5704 such as CB[7] is low, as this is shown in the diagram in the lower part of FIG. 60. As a result, hybridization occurs, and the analyte 5350 is labeled with the dye 5312a.

In step S2′, the concentration of the host molecule 5704 is increased so that dissociation occurs. As a result, the reporter 5316a including the dye 5312a is removed from the analyte 5350.

In step S3′, the sample is stained again. However, an oligonucleotide probe is used that is formed from a reporter 5316b having a dye 5312b which is different from dye 5312a applied in the step S1′. After staining, the sample is imaged to create a further readout. While performing step S3′, the concentration of the host molecule 5704 is low again. As a result, hybridization occurs, and the analyte 5350 is labeled with the dye 5312b.

The steps of staining the sample, imaging the sample and removing the labels can be repeated to implement a cyclic process.

FIG. 61 shows schematically how unmodified DNA oligonucleotides, harboring at least one GG mismatch, can be used together with a photoswitchable molecular glue called NCDA and 360 nm and/or 430 nm illumination to enable either repeated or dynamic association/dissociation (binding/unbinding) between the “oligonucleotide attachment portion” 5306a/“A′” conjugated to a combinatorial label and the “oligonucleotide barcode” 5304a/“A” conjugated to an assigned affinity reagent. The “oligonucleotide barcode” 5304a/“A” may in the case of a nucleic acid analyte like DNA or RNA simply be a cognate nucleic acid target site.

As illustrated in FIG. 61 the at least one GG mismatch inhibits the stable association between “oligonucleotide attachment portion” 5306a/“A′” conjugated to a combinatorial label and the “oligonucleotide barcode” 5304a/“A” conjugated to an assigned affinity reagent in the absence of cis-NCDA and/or in the presence of trans-NCDA. Photoswitching trans-NCDA to cis-NCDA, however, allows the at least one GG mismatch to be bridged by with guanine-recognizing naphthyridine moieties which enables hybridization despite the GG mismatch. Cis/trans-isomerization of NCDA is reversibly controlled via photoswitching using 360 nm light to generate cis and 430 nm light illumination to generate trans isoform. The binding kinetics can therefore be either indirectly light controlled by shifting the concentrations of cis- and trans-NCDA or by supplying cis-NCDA and/or trans-NCDA externally using buffer exchanges. A particular advantage of the NCDA-approach is the fact, that oligonucleotide attachment portions” 5306a/“A′” and “oligonucleotide barcodes” 5304a may consists only of natural bases, so no potentially labor- and cost-intensive modifications are needed on the side of the barcode. Likewise, rapid photoswitching by alternating 360 nm/430 nm light illumination can be used in conjunction with buffers that have NCDA to put the system in a state, where the oligonucleotide-barcodes of affinity reagents dynamically associate and dissociate with the attachment portions of the linkers of the reporters/combinatorial labels. The use of photoswitchable glue for DNA is described in Dohno et al. 2007 (J. Am. Chem. Soc. 2007, 129, 39, 11898-11899).

Swift buffer exchanges to raise or lower concentrations of salt or small molecules can be performed intra-round as the dissociation constant of the much larger affinity reagents and combinatorial labels/reporters avoids their rapid washout. Likewise, a continuous resupply of combinatorial labels with buffers of varying concentrations of salts, small molecules, or other additives is possible. In this way concentration profiles of salts, small molecules and other constituents of the buffer can be run intra-round, i.e. in the first incomplete round consisting of staining-imaging. This consideration applies to all strategies referenced in this document that can be used for intra-round code swapping.

A′ and B′ are therefore either fully or partially complementary to A and B respectively (i.e. A is configured to bind to A′ and B to B′ in a stable, but reversible, or transient fashion). A 5304a, B 5304b, A′ 5306a, and B′ 5360b may include mismatches, in particular GG mismatches, or hairpins, i.e. stretches that do not hybridize, and may be fully or partially comprised of natural or non-natural nucleobases or nucleosides. Nucleobases may be modified with further moieties such as the following guest molecules:
1-adamantanemethylamine (AD),
ferrocenyl methylamine (FC),
1,4-benzenedimethanamine (BA)
4-tert-butylbenzylamine (TB)

In a particularly preferred embodiment, “oligonucleotide barcodes” 5304a and/or oligonucleotide attachment portions” 5306a are being used that comprise one of the following structures

5′-XXXXXX C* XXXXXX C* XXXXX-3′
5′-X..X C* X..X C* X..X-3′

wherein X may be any nucleobase or nucleoside natural or non-natural and C* refers to a guest-modified cytosine molecule nucleoside. C* may further refer to any other guest-modified nucleoside.

As used herein the term “and/or” includes any and all combinations of one or more of the associated listed items and may be abbreviated as “/”.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

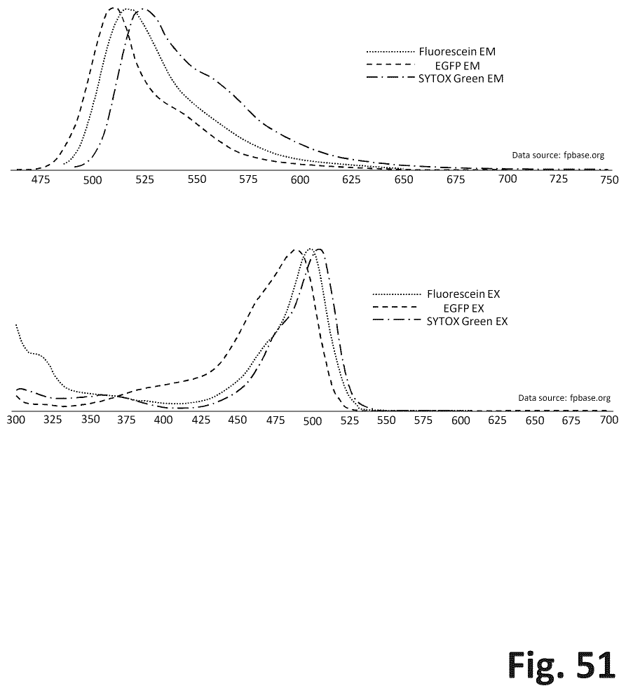

Some embodiments relate to a microscope comprising a system as described in connection with one or more of the FIGS. 1 to 51. Alternatively, a microscope may be part of or connected to a system as described in connection with one or more of the FIGS. 1 to 51. FIGS. 50A and 50B as well as FIGS. 23-27 show a schematic illustrations of a readout device or system 5000 configured to perform a method described herein. The readout device or system 5000 comprises a microscope or a cytometer 2500 and a computer system 5016 connected by suitable communication protocols and connections 5018 (e.g. USB, TCP/IP, Ethernet, FibreChannel, CAN-Bus). The microscope 5014 could e.g. be used for generating an optical readout of a marker and is configured to take images and is connected to the computer system 5016. The computer system 5016 is configured to execute at least a part of a method described herein. The computer system 5016 may be configured to execute a machine learning algorithm. The computer system 5016 and microscope or a cytometer 2500 may be separate entities but can also be integrated together in one common housing. The computer system 5016 may be part of a central processing system of the microscope or a cytometer 2500 and/or the computer system 5016 may be part of a subcomponent of the microscope or a cytometer 2500, such as a sensor, an actor, a camera or an illumination unit, etc. of the microscope or a cytometer 2500.

The computer system 5016 may be a local computer device (e.g. personal computer, laptop, tablet computer or mobile phone) with one or more processors and one or more storage devices or may be a distributed computer system (e.g. a cloud computing system with one or more processors and one or more storage devices distributed at various locations, for example, at a local client and/or one or more remote server farms and/or data centers). The computer system 5016 may comprise any circuit or combination of circuits. In one embodiment, the computer system 5016 may include one or more processors which can be of any type. As used herein, processor may mean any type of computational circuit, such as but not limited to a microprocessor, a microcontroller, a complex instruction set computing (CISC) microprocessor, a reduced instruction set computing (RISC) microprocessor, a very long instruction word (VLIW) microprocessor, a graphics processor, a digital signal processor (DSP), multiple core processor, a field programmable gate array (FPGA), for example, of a microscope or a microscope component (e.g. camera) or any other type of processor or processing circuit. Other types of circuits that may be included in the computer system 5016 may be a custom circuit, an application-specific integrated circuit (ASlC), or the like, such as, for example, one or more circuits (such as a communication circuit) for use in wireless devices like mobile telephones, tablet computers, laptop computers, two-way radios, and similar electronic systems. The computer system 5016 may include one or more storage devices, which may include one or more memory elements suitable to the particular application, such as a main memory in the form of random access memory (RAM), one or more hard drives, and/or one or more drives that handle removable media such as compact disks (CD), flash memory cards, digital video disk (DVD), and the like. The computer system 5016 may also include a display device, one or more speakers, and a keyboard and/or controller, which can include a mouse, trackball, touch screen, voice-recognition device, or any other device that permits a system user to input information into and receive information from the computer system 5016.

Some or all of the method steps may be executed by (or using) a hardware apparatus, such as, a processor, a microprocessor, a programmable computer or an electronic circuit. In some embodiments, some one or more of the most important method steps may be executed by such an apparatus.

Depending on certain implementation requirements, embodiments of the invention can be implemented in hardware or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments according to the invention comprise a data carrier having electronically readable control signals, which are capable of cooperating with a programmable computer system, such that one of the methods described herein is performed.

Generally, embodiments of the present invention can be implemented as a computer program product with a program code, the program code being operative for performing one of the methods when the computer program product runs on a computer. The program code may, for example, be stored on a machine readable carrier.

Other embodiments comprise the computer program for performing one of the methods described herein, stored on a machine readable carrier.

In other words, an embodiment of the present invention is, therefore, a computer program having a program code for performing one of the methods described herein, when the computer program runs on a computer.

A further embodiment of the present invention is, therefore, a storage medium (or a data carrier, or a computer-readable medium) comprising, stored thereon, the computer program for performing one of the methods described herein when it is performed by a processor. The data carrier, the digital storage medium or the recorded medium are typically tangible and/or non-transitionary. A further embodiment of the present invention is an apparatus as described herein comprising a processor and the storage medium.

A further embodiment of the invention is, therefore, a data stream or a sequence of signals representing the computer program for performing one of the methods described herein. The data stream or the sequence of signals may, for example, be configured to be transferred via a data communication connection, for example, via the internet.

A further embodiment comprises a processing means, for example, a computer or a programmable logic device, configured to, or adapted to, perform one of the methods described herein.

A further embodiment comprises a computer having installed thereon the computer program for performing one of the methods described herein.

A further embodiment according to the invention comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE SIGNS

100, 100a, 100b, 100A to 100E Fluorescent dye
100M, 100N, 100O, 100P SMILEs
102, 102a Left half pattern resembles excitation properties
104, 104a Right half pattern resembles emission properties
106 Dye-conjugated or oligonucleotide-labeled primary affinity reagents
108 Single-domain antibody
110 Multimerized (single-domain) antibody
112 Antibody
114 Aptamer
116 Oligonucleotide
118 Toxin, drug, small molecule
120 Oligonucleotide barcode
124 (108-118) without attached dye or oligonucleotide
200 Fluorescence light
202 Spectrally separated fluorescence light
204 Prism
206 Grating
208 Detector with multiple detector elements
300 Plurality of excitation spectra
302 Plurality of emission spectra
304a, 304b, 304c □ gates of □ classes
306 Group of dyes with similar emission spectra
308a, 308b, 308c Emission spectrum
400 Topographical fluorescence lifetime-emission intensity plots
402a, 402b, 402c Dyes A1, A2, A3
404 Spectral phasor, FLIM phasor, or Spectral-FLIM phasor
500 Reactivity or specificity-determining region of the affinity reagent (e.g. paratope)
600 Sets of dyes A to n
602, 602a, 602b, 602c, 602d, 706, 706a, 706b, 706c, 706d Combination of dyes
604 Pair of a combination of dyes (si) and an affinity reagent (ai) (bijective; one-to-one correspondence)
606a, 606b Mapping of a combination of dyes (si) onto an affinity reagent (ai) or vice versa (injective)
700 digit of (704a, 704b)
702 Plurality of dyes (YD)
704a, 704b Rule of forming a combination of dyes/binary code
706 code blocks/code segments
708 Marker, μ
710 Acquisition sequence
800a, 800b, 800c Oligonucleotide barcode sequence
900 Target molecule or analyte
902 Linker
904 Cleavage site
906a, 906b, 906c, 906d, 906e, Complementary sites/sequences
906a*, 906b*, 906c*, 906d*, 906e* Attachment site/sequences
908, 1008a, 1008b Reporter
1500, 1500a Cell
1502 Nucleus
1504 Cytoplasm
1506a, 1506b, 1506c Target protein
1508 DNA target sequence
1510 RNA target sequence
1512 Primary antibody (non-labeled)
1514 Secondary antibody (labeled)
1518 Primary single-domain antibody (non-labeled)
1520a, 1520b Oligonucleotide-based reporter
1522 Capture antibody immobilized in the extracellular space
1524a, 1524b, 1524c Secreted target protein
1526a, 1526b, 1526c, 1526d Detection antibody
1600 Atom
1602 Small molecule (e.g. cholesterol, a drug)
1604 Nanobody
1606 Green fluorescent protein
1608 Antibody
1610 Quantum dot, polymer dot, nanostructure
1700 Point spread function (schematic)
1900 Discrete entity (e.g. hydrogel bead) or virtual space surrounding a cell of interest (e.g. in a well of a microplate); virtual or physical volume
1902a, 1902b Capture reagents
1904 Polymer (e.g. hydrogel)
2000, 2000a Micro-/Nanobead (e.g. latex or polystyrene bead)
2002 Nanostructure (e.g. nanoruler, DNA-origami, carbontube)
2300 Microplate or sample carrier
2302 Well or sample container
2304 Transparent window
2306 Hydrogel or transparent polymer
2308 Center of mass of cell or structure of interest
2310 Virtual volume surrounding center of mass
2312 Microscope, imaging system, image-based readout device
2314 Light source
2316 Beam path, beam splitter, beam routing
2320 Detector
2318 Imaging optic or objective
2400 Flow Cell
2402 Inflow
2404 Outflow
2406 Flow medium
2408 Discrete entity (e.g. hydrogel bead)
2500 Flow cytometer
2502 Flow sorter
2504 Deflector plates
2506 Collection tubes
2600 Bead-based analyte detection assay
2602 Bead-based analyte interaction assay
2700 Cell-surface bound target
2800 Spot
2802a, 2802b Mono-species readout volume
2804 Multi-species spot
2900 Readout sequence
3200 Plurality of combinations of dyes (S1)
3202 Set of combinations of dyes subsumable under the first readout sequence
3300a, 3300b, 3300c Set of combinations of dyes not-subsumable under the first readout sequence
3302a, 3302b, 3302c Set of combinations of dyes subsumable under the first readout sequence
3400a, 3400b, 3400c Set of combinations of dyes corresponding to markers present in the readout spot or readout volume
3402a, 3402b, 3402c Set of combinations of dyes subsumable under the first readout sequence but corresponding to markers not present in the readout spot or readout volume
3800 Primary qualitative decoding
3802 Secondary qualitative decoding
3804 Secondary quantitative decoding
3850 Backbone
3900 Unspecific covalent coupling or high affinity binding site
4100e, 4100h, 4100j Dye-specific or dye-conjugated affinity tag/ligand-specific covalent coupling or high affinity binding site
4102e, 4102h, 4102j Dye-conjugated affinity tag/ligand
4300 Microcapsule/nanocapsule filled with mix of dyes
4302 Microbead/nanobead filled with mix of dyes
4400 Combination of SMILES on a reporter
4500 Nanoplatform carrying dyes (e.g. SMILES) based on a nanostructure (e.g. nanoruler, DNA-origami, carbontubes)
4600 Combination of SMILES embedded in a polymer microbead
4700 Combination of SMILES encapsulated in a microcapsule
4800 Oligonucleotide-based linker
4802 Combined oligonucleotide- and peptide-based linker
4804 Combined oligonucleotide- and nanoruler/DNA-origami-based linker
4806 Peptide linker
4808 Combined oligonucleotide-microbead linker
4900 Affinity ligand
4902 Affinity tag
4904 Secondary nanobodies
4906 Aptamer
5100 Cohort of strongly overlapping excitation spectra
5102 Cohort of strongly overlapping emission spectra
5700 Modified nucleoside
5702 Guest molecule
5704 Host molecule
5312 Dye
5314 Dye
5316 Reporter
5318 Marker

1-37. (canceled)

38: A linker, configured to bind to an affinity reagent, the linker comprising:
at least one binding site configured to bind at least one dye; and
an oligonucleotide attachment portion configured to bind to an oligonucleotide barcode of the affinity reagent.

39: The linker according to claim 38, comprising:
a plurality of binding sites, wherein at least two of the plurality of binding sites are configured to bind to dyes having different characteristics for at least one of: excitation or emission.

40: A reporter, comprising:
a linker according to claim 38; and
the at least one dye bound to the at least one binding site of the linker.

41: A reporter, comprising:
a linker according to claim 39; and
a combination of dyes, each respective dye bound to a respective one of the plurality of binding sites of the linker, wherein at least two of the combination of dyes have different characteristics for at least one of: excitation or emission.

42: A marker, comprising:
a reporter according to claim 40; and
the affinity reagent attach to an analyte, wherein the reporter is attached to the affinity reagent.

43: The marker according to claim 42, wherein the oligonucleotide attachment portion of the linker is fully or partially complementary to the oligonucleotide barcode of the affinity reagent.

44: The marker according to claim 42, wherein the oligonucleotide attachment portion of the linker is configured to have an affinity to the oligonucleotide barcode of the affinity reagent so as to bind transiently under at least one condition before dissociating again from the oligonucleotide barcode,
wherein the at least one condition provides for dissociation of the oligonucleotide attachment portion depending on at least one of host-guest mediated ligand-invasion, strand displacement, or photochemical control of hybridization.

45: The marker according to claim 42, wherein one of the oligonucleotide attachment portion of the linker and the oligonucleotide barcode of the affinity reagent comprises at least one guest molecule attached to a first nucleobase thereof and configured to complexate with a host molecule, and
wherein the other of the oligonucleotide attachment portion of the linker and the oligonucleotide barcode of the affinity reagent comprises at least one nucleobase complementary to the first nucleobase to which the guest molecule is attached.

46: The marker according to claim 45, wherein the host molecule is a cucurbituril.

47: The marker according to claim 42, comprising at least one base mismatch between the oligonucleotide attachment portion of the linker and the oligonucleotide barcode of the affinity reagent hindering stable association therebetween,
wherein the at least one base mismatch is configured to be bridged by photoswitching.

48: The marker according to claim 47, wherein the at least one base mismatch is a GG mismatch, which is configured to be bridged by photoswitching trans-NCDA to cis-NCDA.

49: The marker according to claim 42, wherein the affinity reagent is an oligonucleotide-probe which is configured to bind to a nucleic acid analyte.

50: The marker according to claim 42, wherein each of the oligonucleotide attachment portion of the linker and the oligonucleotide barcode of the affinity reagent has a length of 9 to 13 nucleotides.

51: A plurality of markers according to claim 42, wherein each respective reporter comprises a respective unique combination of dyes, and wherein each respective reporter is attached to a respective affinity reagent configured for attachment to an analyte, such that no unique combination of dyes is associated with more than one affinity reagent.

52: A method for analyzing a sample, the sample comprising:
a plurality of affinity reagents, each respective affinity reagent being configured to attach to a respective analyte, at least one of the plurality of affinity reagents being attached to an analyte; and
a first plurality of combinations of dyes, each respective combination of dyes being unique among the first plurality of combinations of dyes, and each respective combination of dyes comprising at least two dyes having different characteristics for at least one of excitation or emission,
wherein at least one of the first plurality of combinations of dyes is attached to an associated affinity reagent according to a first mapping,
wherein at least another one of the first plurality of combinations of dyes is attached to the associated affinity reagent according to a second mapping,
wherein the first mapping is different from the second mapping,
the method comprising, for the first mapping and for the second mapping in a time sequential manner:
i) directing excitation light at the sample, the excitation light having characteristics for exciting the at least two dyes having different characteristics for at least one of: excitation or emission; and
ii) generating at least one first readout from emission light emitted by the at least two dyes excited by excitation light.

53: A method for analyzing a sample, the sample comprising:
a plurality of affinity reagents, each respective affinity reagent being configured to attach to a respective analyte, at least one of the plurality of affinity reagents being attached to an analyte; and
a first plurality of combinations of dyes, each combination of dyes of the first plurality of combinations of dyes being unique among the first plurality of combinations of dyes and comprising at least two dyes having different characteristics for at least one of: excitation or emission,
a second plurality of combinations of dyes, each combination of dyes of the second plurality of combination of dyes being unique among the second plurality of combinations of dyes and comprising at least two dyes having different characteristics for at least one of excitation or emission,
wherein the second plurality of combinations of dyes is different from the first plurality of combinations of dyes,
wherein at least one of the first plurality of combinations of dyes is attached to an associated affinity reagent according to a first mapping,
wherein at least one of the second plurality of combinations of dyes is attached to the associated affinity reagent according to a second mapping,
wherein the first mapping is different from the second mapping,
the method comprising, for the first mapping and for the second mapping in a time sequential manner:
i) directing excitation light at the sample, the excitation light having characteristics for exciting the at least two dyes having different characteristics for at least one of: excitation or emission; and
ii) generating at least one first readout from emission light emitted by the at least two dyes excited by the excitation light.

54: The method according to claim 52, wherein each combination of dyes of the first plurality of combinations of dyes is attached to only one affinity reagent, such that no combination of dyes is associated with more than one affinity reagent in the first mapping.

55: The method according to claim 52, further comprising at least one of deactivating at least one dye in the first plurality of combinations of dyes;
removing an attachment between at least one affinity reagent and at least one of the first plurality of combinations of dyes;
removing an attachment between at least one affinity reagent and at least one of the analytes; or
waiting longer than a fluorescence lifetime of at least one dye in the first plurality of combinations of dyes, and repeating steps i) and ii) for the first plurality of combinations of dyes according to the second mapping.

56: The method according to claim 52, wherein the at least another one of the first plurality of combinations of dyes is stochastically attached to the associated affinity reagent according to the second mapping, and wherein stochastic labeling is achieved by at least one of DNA-PAINT, DNA-Exchange, Exchange-PAINT, or by using oligonucleotide barcodes attached to the affinity reagents and complementary attachment oligonucleotide portions attached to the first plurality of combinations of dyes, which are configured to allow repeated association and dissociation of the first plurality of combinations of dyes and the affinity reagents by at least one of the following:
a change of concentration of a host molecule;
a change of temperature;
a change of illumination condition;
a change in a concentration of at least one capture strand; or
a change in a concentration and/or activity of an enzyme.

57: The method according to claim 52, further comprising:
generating a second readout from the emission light; and
determining, by at least one computer processor, at least a first affinity reagent present in the sample, by:
comparing the at least one first readout and the second readout; and
determining a presence of the first affinity reagent based, at least in part, on the comparison.

58: A solution comprising a linker according to claim 38.

59: A lyophilized solid comprising a linker according to claim 38.
